(19) United States
(12) Patent Application Publication
(10) Pub. No.: US 2024/0199691 A1
(43) Pub. Date: Jun. 20, 2024

(54) EXPRESSION AND PURIFICATION OF CAS ENZYMES

(71) Applicant: INTEGRATED DNA TECHNOLOGIES, INC., Coralville, IA (US)

(72) Inventors: Sarah Franz BEAUDOIN, Coralville, IA (US); Michael Allen COLLINGWOOD, Coralville, IA (US); Christopher Anthony VAKULSKAS, Coralville, IA (US); Mark Aaron BEHLKE, Coralville, IA (US)

(21) Appl. No.: 18/416,017

(22) Filed: Jan. 18, 2024

Related U.S. Application Data
(62) Division of application No. 17/185,788, filed on Feb. 25, 2021, now Pat. No. 11,970,516.
(60) Provisional application No. 62/982,231, filed on Feb. 27, 2020.

(51) Int. Cl. C07K 1/36; C07K 1/18; C07K 1/22; C12N 9/22

(52) U.S. Cl. CPC C07K 1/36; C07K 1/18; C07K 1/22; C12N 9/22; C12Y 301/00

(57) ABSTRACT

Described herein are methods for the expression and purification of Cas13a and methods for detecting target RNA using Cas13a.

### CROSS-REFERENCE TO RELATED APPLICATION(S)

This application is a divisional of U.S. patent application Ser. No. 17/185,788, filed on Feb. 25, 2021, which claims priority to U.S. Provisional Patent Application No. 62/982,231, filed on Feb. 27, 2020, each of which is incorporated by reference herein in its entirety.

### REFERENCE TO SEQUENCE LISTING

This application was filed with a Sequence Listing in ST.26 XML format accordance with 37 C.F.R. § 1.831. The Sequence Listing XML file submitted in the USPTO Patent Center, “013670-9065-US03_sequence_listing_XML_18 Jan. 2024.xml,” was created on Jan. 18, 2024, contains 44 sequences, has a file size of 169 Kbytes, and is incorporated by reference in its entirety into the specification.

### TECHNICAL FIELD

Described herein are methods for the expression and purification of Cas13a and methods for detecting target RNA using Cas13a.

### BACKGROUND

The RNA targeting enzyme family Cas13 is a CRISPR system identified in an effort to identify new CRISPR systems in addition to Cas9 and Cas12a (also referred to as Cpf1). Cas13 has four subtypes (Cas13a-d) and Cas13a (formerly known as C2c2) is a single effector protein that lacks homology with any known DNA nuclease; however, the protein contains two Higher Eukaryotes and Prokaryotes Nucleotide-binding (HEPN) domains that more commonly function as ribonucleases (RNases). Abudayyeh et al., demonstrated that Cas13a could act as an RNA-directed RNase [1].

Cas13a is classified as a class 2 type VI Clustered Regularly Interspaced Short Palindromic Repeat (CRISPR) adaptive immune system protein that provides direct cleavage of RNA when complexed with a CRISPR RNA (crRNA). This complex is called a CRISPR ribonucleoprotein (RNP) complex. Once the Cas13a RNP recognizes and cleaves its RNA target, the protein engages in collateral cleavage of nonspecific RNAs. For this reason, Cas13a can provide specific RNA sensing in vitro by utilizing its nonspecific RNase activity in the degradation of fluorescent-labeled RNA. This system has led to the rapid and inexpensive detection of nucleic acids by Cas13a and can be applied in disease diagnostics and epidemiology by detecting single RNA molecules with high specificity.

A method for nucleic acid detection by Cas13a RNP is described by Gootenberg et al., using Leptotrichia wadei (Lwa) Cas13a and denoted as SHERLOCK (Specific High Sensitivity Enzymatic Reporter UnLOCKing) [2]. Gootenberg et al. describe LwaCas13a as a superior protein over both Leptotrichia buccalis (Lbu) and Leptotrichia shahii (Lsh) species, as it yields detection sensitivity of approximately 50 fM. They surveyed the applications of the SHERLOCK technology towards infectious diseases, bacterial pathogens, low frequency cancer mutations in cell free DNA fragments, among others. For instance, they could discriminate between the Zika virus and the related flavivirus, Dengue, down to 2 aM. The SHERLOCK technology is a sensitive nucleic acid detection that can easily be applied for field applications.

The purification of LwaCas13a, as described by Gootenberg et al., consists of four purification steps: affinity chromatography, followed by removal of the 6×His/Twin Strep by SUMO digestion, cation exchange chromatography and finally, gel filtration chromatography [2].

What is needed is a simplified process for the expression and purification of Cas13 proteins.

### SUMMARY

One embodiment described herein is a method for expressing and purifying a Cas13a protein, the method comprising: (a) inserting a nucleotide sequence encoding polypeptides having 95-99% identity to polypeptide sequences of SEQ ID NO: 2, 4, 6, 8, 10, 12, or 14 into an expression plasmid; (b) transforming one or more cells with the expression plasmid; (c) inducing expression of the transformed plasmid; (d) isolating the cells; (e) extracting the Cas13a protein; and (f) purifying the protein using affinity purification and ion exchange purification. In one aspect, the Cas13a protein comprises one or more of Leptotrichia buccalis (Lbu), Leptotrichia shahii (Lsh), and Leptotrichia wadei (Lwa) Cas13a proteins, or mutants thereof. In another aspect, the nucleotide sequence has 90-99% identity to SEQ ID NO: 1, 3, 5, 7, 9, 11, or 13. In another aspect, the nucleotide sequence is selected from SEQ ID NO: 1, 3, 5, 7, 9, 11, or 13. In another aspect, the encoded polypeptides are selected from SEQ ID NO: 2, 4, 6, 8, 10, 12, or 14. In another aspect, the cell comprises E. coli BL21(DE3). In another aspect, the expression plasmid comprises pET28 or pET28-MBP-TEV plasmids. In another aspect, the nucleotide sequence is inserted into the expression plasmid using isothermal assembly. In another aspect, the affinity purification comprises a nickel or a maltose affinity media.

In one aspect, the affinity purification comprises affinity chromatography comprising: (a) equilibrating a nickel affinity column with a binding buffer and loading the extracted Cas13a protein; (b) washing the nickel affinity column with a wash buffer; and (c) eluting the affinity purified Cas13a protein from the nickel affinity column using an elution buffer.

In one aspect, the affinity purification comprises affinity chromatography comprising: (a) equilibrating a maltose affinity column with a binding buffer and loading the extracted Cas13a protein; (b) washing the maltose affinity column with a wash buffer; and (c) eluting the affinity purified Cas13a protein from the maltose affinity column using an elution buffer. In another aspect, the ion exchange purification comprises a cation exchange media.

In one aspect, the ion exchange purification comprises cation exchange chromatography comprising: (a) equilibrating a cation exchange column with a binding buffer and loading the extracted Cas13a protein; (b) washing the cation exchange column with a wash buffer; and (c) eluting the cation exchange purified Cas13a protein from the cation exchange column using an elution buffer. In another aspect, the method further comprises concentrating the purified Cas13a protein to approximately 10 mg/mL. In another aspect, the method further comprises dialyzing the concentrated purified Cas13a protein.

Another embodiment described herein is a method for purifying a recombinant Cas13a protein, the method comprising: (a) providing an expressed recombinant Cas13a protein having 95-99% identity to the polypeptide sequences of SEQ ID NO: 2, 4, 6, 8, 10, 12, or 14; (b) performing an affinity purification comprising a nickel affinity media; (c) performing an affinity purification comprising maltose affinity media; (d) performing an ion exchange purification comprising a cation exchange media; and (e) collecting the purified Cas12 protein. In another aspect, the Cas13a proteins are encoded by a nucleotide sequence having 90-99% to SEQ ID NO: 1, 3, 5, 7, 9, 11, or 13. In another aspect, the Cas13a proteins are encoded by a nucleotide sequence of SEQ ID NO: 1, 3, 5, 7, 9, 11, or 13. In another aspect, the Cas13a proteins are selected from polypeptide sequences of SEQ ID NO: 2, 4, 6, 8, 10, 12, or 14. In another aspect, the method further comprises comprising concentrating the purified Cas13a protein to approximately 10 mg/mL. In another aspect, the method further comprises dialyzing the concentrated purified Cas13a protein against three rounds of dialysis buffer.

Another embodiment described herein is a nucleic acid detection system comprising: a Cas13a protein; one or more guide RNA designed to hybridize to a corresponding target nucleic acid; and a degradation reporter probe. In one aspect, the Cas13a protein is selected from the group comprising Lwa Cas13a, Lbu Cas13a, or Lsh Cas13a. In another aspect, the Lwa Cas13a or Lbu Cas13a is present at a concentration of 0.98 nM to 1000 nM. In another aspect, the Lbu Cas13a is present at a concentration of 0.98 nM to 1000 nM. In another aspect, the Lbu Cas13a is present at a concentration of 3.91 nM to 31.3 nM. In another aspect, the degradation reporter probe is fluorescently labeled.

Another embodiment described herein is a method of detecting a target nucleic acid comprising: (a) providing a Cas13a protein; (b) one or more guide RNA designed to hybridize to a corresponding target nucleic acid; and (c) a degradation reporter probe; wherein the Cas13a protein is present at an effective concentration to promote cleavage of the corresponding target nucleic acid and the degradation reporter probe to generate a detectable signal. In one aspect, the detectable signal is a fluorescent signal.

### BRIEF DESCRIPTION OF THE DRAWINGS

FIG. 1 shows an SDS-PAGE indicating the purity of Cas13a variants after the final step in purification and dialysis into storage buffer.

FIG. 2A shows the nucleic acid target sequence with the complementary sequence bolded.

FIG. 2B shows the nucleic acid target and crRNA interactions (bold).

FIG. 3A shows a fluorescent emission of titrated LbuCas13a ribonucleoprotein complex (RNP). FIG. 3B shows a closeup of the same data in FIG. 3A illustrating a bell-like curve with an optimum RNP concentration range between 4 and 31 nM.

FIG. 4 shows the fluorescent emission of a cleaved RNA reporter by Cas13a variants at different enzyme concentrations.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present document, including definitions, will control. Representative compositions, methods, and materials are described herein, although equivalent materials and methods can be used in practice.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

The methods and compositions escribed herein can be used with any CRISPR system wherein the Cas nuclease targets RNA. In one embodiment, the methods described herein utilize Cas13 enzyme. In another embodiment the Cas13 enzyme is a Cas13a subtype. There are two distinct subfamilies of the Cas13a protein family, adenosine (A) or uridine (U) cleaving. In another embodiment described herein, the methods utilize a LbuCas13a, a single effector RNA-directed RNase, an example being a LbuCas13a from the Leptotrichia buccalis CRISPR adaptive immune system, which resides in the uridine (U) cleaving subfamily of Cas13a proteins. The ability of LbuCas13a to act as a non-specific RNase was described by East-Seletsky et al. and showed that this class of enzymes is capable of two RNA cleavage activities: crRNA-mediated cleavage of target RNA, followed by non-specific RNase activity [3].

The purification of Cas13a has been described by both Gootenberg et al. [2], and East-Seletsky et al. [3] and consists of four purification steps each. Gootenberg et al. [2] describes the overexpression of LwaCas13a from a pET SUMO expression plasmid. The purification begins with affinity chromatography by StrepTactin® Sepharose (IBL Lifesciences), followed by removal of the 6×His/Twin Strep by SUMO digestion. The native protein is further purified by cation exchange chromatography (HiTrap™ SP HP) and gel filtration chromatography (Superdex® 200).

The purification described by East-Seletsky et al. [3] uses a similar procedure, except that LbuCas13a is N-terminally expressed with a 6×His-MBP-TEV tag. The purification procedure consists of affinity chromatography, removal of 6×His-MBP by TEV protease, cation exchange chromatography with a HiTrap™ SP column (Cytiva) and gel filtration chromatography (Superdex® 200).

The methods described herein simplify the purification process by only using two steps: affinity chromatography and cation exchange chromatography. The purification protocol leaves the 6×HisTag (CTD) intact while not sacrificing activity. Unlike previous methods which use 45 nM purified LwaCas13a with 22.5 nM crRNA to form the RNP complex, the current method utilizes LbuCas13a and a 10-fold reduction of purified protein (4 nM) with an equal concentration of crRNA.

One embodiment described herein is a method for expressing and purifying a Cas13a protein, the method comprising: (a) inserting a nucleotide sequence encoding polypeptides having 95-99% identity to polypeptide sequences of SEQ ID NO: 2, 4, 6, 8, 10, 12, or 14 into an expression plasmid; (b) transforming one or more cells with the expression plasmid; (c) inducing expression of the transformed plasmid; (d) isolating the cells; (e) extracting the Cas13a protein; and (f) purifying the protein using affinity purification and ion exchange purification. In one aspect, the Cas13a protein comprises one or more of Leptotrichia buccalis (Lbu), Leptotrichia shahii (Lsh), and Leptotrichia wadei (Lwa) Cas13a proteins, or mutants thereof. In another aspect, the nucleotide sequence has 90-99% identity to SEQ ID NO: 1, 3, 5, 7, 9, 11, or 13. In another aspect, the nucleotide sequence is selected from SEQ ID NO: 1, 3, 5, 7, 9, 11, or 13. In another aspect, the encoded polypeptides are selected from SEQ ID NO: 2, 4, 6, 8, 10, 12, or 14. In another aspect, the cell comprises E. coli BL21(DE3). In another aspect, the expression plasmid comprises pET28 or pMAL plasmids. In another aspect, the nucleotide sequence is inserted into the expression plasmid using isothermal assembly. In another aspect, the affinity purification comprises a nickel or a maltose affinity media.

In one aspect, the affinity purification comprises affinity chromatography comprising: (a) equilibrating a nickel affinity column with a binding buffer and loading the extracted Cas13a protein; (b) washing the nickel affinity column with a wash buffer; and (c) eluting the affinity purified Cas13a protein from the nickel affinity column using an elution buffer.

In one aspect, the affinity purification comprises affinity chromatography comprising: (a) equilibrating a maltose affinity column with a binding buffer and loading the extracted Cas13a protein; (b) washing the maltose affinity column with a wash buffer; and (c) eluting the affinity purified Cas13a protein from the maltose affinity column using an elution buffer. In another aspect, the ion exchange purification comprises a cation exchange media.

In one aspect, the ion exchange purification comprises cation exchange chromatography comprising: (a) equilibrating a cation exchange column with a binding buffer and loading the extracted Cas13a protein; (b) washing the cation exchange column with a wash buffer; and (c) eluting the cation exchange purified Cas13a protein from the cation exchange column using an elution buffer. In another aspect, the method further comprises concentrating the purified Cas13a protein to approximately 10 mg/mL. In another aspect, the method further comprises dialyzing the concentrated purified Cas13a protein.

Another embodiment described herein is a method for purifying a recombinant Cas13a protein, the method comprising: (a) providing an expressed recombinant Cas13a protein having 95-99% identity to the polypeptide sequences of SEQ ID NO: 2, 4, 6, 8, 10, 12, or 14; (b) performing an affinity purification comprising a nickel affinity media; (c) performing an affinity purification comprising maltose affinity media; (d) performing an ion exchange purification comprising a cation exchange media; and (e) collecting the purified Cas12 protein. In another aspect, the Cas13a proteins are encoded by a nucleotide sequence having 90-99% to SEQ ID NO: 1, 3, 5, 7, 9, 11, or 13. In another aspect, the Cas13a proteins are encoded by a nucleotide sequence of SEQ ID NO: 1, 3, 5, 7, 9, 11, or 13. In another aspect, the Cas13a proteins are selected from polypeptide sequences of SEQ ID NO: 2, 4, 6, 8, 10, 12, or 14. In another aspect, the method further comprises comprising concentrating the purified Cas13a protein to approximately 10 mg/mL. In another aspect, the method further comprises dialyzing the concentrated purified Cas13a protein against three rounds of dialysis buffer.

Another embodiment described herein is a nucleic acid detection system comprising: a Cas13a protein; one or more guide RNA designed to hybridize to a corresponding target nucleic acid; and a degradation reporter probe. In one aspect, the Cas13a protein is selected from the group comprising Lwa Cas13a, Lbu Cas13a, or Lsh Cas13a. In another aspect, the Lwa Cas13a or Lbu Cas13a is present at a concentration of 0.98 nM to 1000 nM. In another aspect, the Lbu Cas13a is present at a concentration of 0.98 nM to 1000 nM. In another aspect, the Lbu Cas13a is present at a concentration of 3.91 nM to 31.3 nM. In another aspect, the degradation reporter probe is fluorescently labeled.

Another embodiment described herein is a method of detecting a target nucleic acid comprising: (a) providing a Cas13a protein; (b) one or more guide RNA designed to hybridize to a corresponding target nucleic acid; and (c) a degradation reporter probe; wherein the Cas13a protein is present at an effective concentration to promote cleavage of the corresponding target nucleic acid and the degradation reporter probe to generate a detectable signal. In one aspect, the detectable signal is a fluorescent signal.

Another embodiment described herein is a polynucleotide vector comprising one or more nucleotide sequences described herein.

Another embodiment described herein is a cell comprising one or more nucleotide sequences described herein or a polynucleotide vector described herein.

Another embodiment is a polypeptide encoded by a nucleotide sequence described herein. In one aspect, the polypeptide has 85% to 99% identity to SEQ ID NO: 2, 4, 6, 8, 10, 12, or 14. In another aspect, the polypeptide is selected from SEQ ID NO: 2, 4, 6, 8, 10, 12, or 14.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence or a polypeptide encoded by the nucleotide sequence produced by the method or the means described herein.

Another embodiment described herein is the use of an effective amount of a polypeptide encoded by one or more of the nucleotide sequences described herein in SEQ ID NO: 1, 3, 5, 7, 9, 11, or 13.

Another embodiment described herein is a research tool comprising a polypeptide encoded by a nucleotide sequence described herein.

Another embodiment described herein is a reagent comprising a polypeptide encoded by a nucleotide sequence described herein.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include (a) nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to nucleotide sequences encoding polypeptide SEQ ID NO: 2, 4, 6, 8, 10, 12, or 14; (b) nucleotide sequences, or degenerate, homologous, or codon-optimized variants thereof, encoding polypeptides having the amino acid sequences in SEQ ID NO: 2, 4, 6, 8, 10, 12, or 14; and (c) nucleotide sequences capable of hybridizing to the complement of any of the nucleotide sequences in (a) or (b) above and capable of expressing functional polypeptides of amino acid sequences in SEQ ID NO: 2, 4, 6, 8, 10, 12, or 14.

By a polynucleotide having a nucleotide sequence at least, for example, 90-99% “identical” to a reference nucleotide sequence encoding a Cas13 protein is intended that the nucleotide sequence of the polynucleotide be identical to the reference sequence except that the polynucleotide sequence can include up to about 10 to 1 point mutations, additions, or deletions per each 100 nucleotides of the reference nucleotide sequence encoding the Cas13 protein.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2: 4 82-489 (1981). This algorithm can be extended to use with peptide sequences using the scoring matrix developed by Dayhoff, Atlas of Protein Sequences and Structure, M. O. Dayhoff ed., 5 suppl. 3: 353-358, National Biomedical Research Foundation, Washington, D.C., USA, and normalized by Gribskov, Nucl. Acids Res. 14(6): 6745-6763 (1986).

For example, due to the degeneracy of the genetic code, one having ordinary skill in the art will recognize that a large number of the nucleic acid molecules having a sequence at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequence shown in SEQ ID NO: 1, 3, 5, 7, 9, 11, or 13, or degenerate, homologous, or codon-optimized variants thereof, will encode a Cas13 protein.

The polynucleotides described herein include those encoding mutations, variations, substitutions, additions, deletions, and particular examples of the polypeptides described herein. For example, guidance concerning how to make phenotypically silent amino acid substitutions is provided in Bowie, J. U. et al., “Deciphering the Message in Protein Sequences: Tolerance to Amino Acid Substitutions,” Science 247: 1306-1310 (1990), wherein the authors indicate that proteins are surprisingly tolerant of amino acid substitutions.

Thus, fragments, derivatives, or analogs of the polypeptides of SEQ ID NO: 2, 4, 6, 8, 10, 12, or 14 can be (i) ones in which one or more of the amino acid residues (e.g., 1, 2, 3, 4, 5, 7, 8, 9, 10, 11, 12, 13, 14, 15, 16, 17, 18, 19, 20, 25, 30, 35, 40, 45, or 50 residues, or even more) are substituted with a conserved or non-conserved amino acid residue (preferably a conserved amino acid residue). Such substituted amino acid residues may or may not be one encoded by the genetic code, or (ii) ones in which one or more of the amino acid residues includes a substituent group (e.g., 1, 2, 3, 4, 5, 7, 8, 9, 10, 15, 20, 25, 30, 35, 40, 45, or 50 residues or even more), or (iii) ones in which the mature polypeptide is fused with another polypeptide or compound, such as a compound to increase the half-life of the polypeptide (for example, polyethylene glycol), or (iv) ones in which the additional amino acids are fused to the mature polypeptide, such as an IgG Fc fusion region peptide or leader or secretory sequence or a sequence which is employed for purification of the mature polypeptide or a proprotein sequence. Such fragments, derivatives, and analogs are deemed to be within the scope of those skilled in the art from the teachings herein.

In addition, fragments, derivatives, or analogs of the polypeptides of SEQ ID NO: 2, 4, 6, 8, 10, 12, or 14 can be substituted with one or more conserved or non-conserved amino acid residue (preferably a conserved amino acid residue). In some cases these polypeptides, fragments, derivatives, or analogs thereof will have a polypeptide sequence at least 50%, 55%, 60%, 65%, 70%, 75%, 80%, 85%, 90%, 91%, 92%, 93%, 94%, 95%, 96%, 97%, 98%, 99% or 100% identical to the polypeptide sequence shown in SEQ ID NO: 2, 4, 6, 8, 10, 12, or 14 and will comprise functional or non-functional proteins or enzymes. Similarly, additions or deletions to the polypeptides can be made either at the N- or C-termini or within non-conserved regions of the polypeptide (which are assumed to be non-critical because they have not been photogenically conserved).

As described herein, in many cases the amino acid substitutions, mutations, additions, or deletions are preferably of a minor nature, such as conservative amino acid substitutions that do not significantly affect the folding or activity of the protein or additions or deletions to the N- or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1.

It will be apparent to one of ordinary skill in the relevant art that suitable modifications and adaptations to the compositions, formulations, methods, processes, apparata, assemblies, and applications described herein can be made without departing from the scope of any embodiments or aspects thereof. The compositions, apparata, assemblies, and methods provided are exemplary and are not intended to limit the scope of any of the disclosed embodiments. All the various embodiments, aspects, and options disclosed herein can be combined in any variations or iterations. The scope of the compositions, formulations, methods, apparata, assemblies, and processes described herein include all actual or potential combinations of embodiments, aspects, options, examples, and preferences described herein. The compositions, formulations, apparata, assemblies, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. All patents and publications cited herein are incorporated by reference herein for the specific teachings thereof.

### REFERENCES

1. Abudayyeh et al., “C2c2 is a single-component programmable RNA-guided RNA-targeting CRISPR effector,” Science 353(6299): aaf5573 (2016).
2. Gootenberg et al., “Nucleic acid detection with CRISPR-Cas13a/C2c2,” Science 356(6336): 438-442 (2017).
3. East-Seletsky et al., “Two distinct RNase activities of CRISPR-C2c2 enable guide-RNA processing and RNA detection,” Nature 538 (7624): 270-273 (2016).
4. Gibson et al., “Enzymatic assembly of DNA molecules up to several hundred kilobases,” Nature Methods 6 (5): 343-34 (2009).

### EXAMPLES

### Example 1

Three Cas13a variants from Leptotrichia buccalis (Lbu), Leptotrichia shahii (Lsh), and Leptotrichia wadei (Lwa) were overexpressed in E. coli cells and purified from lysates thereof. See Table 1. The genes encoding the Lbu, Lsh, and Lwa Cas13a variants were synthesized as gBlocks® Gene Fragments (Integrated DNA Technologies) and inserted into pET28b (SEQ ID NO: 43) and pET28-MBP-TEV (SEQ ID NO: 44) expression plasmids by isothermal assembly of DNA fragments (see [4]) (Table 2). All primers were manufactured by Integrated DNA Technologies Inc.

TABLE 1
Polynucleotide and Polypeptide Sequences of Cas Constructs

LbuCas13a CTD-His Polynucleotide Sequence SEQ ID NO: 1
ATGAAGGTGACCAAAGTTGGTGGTATCAGCCATAAAAAGTATACCAGCGAAGGTCGTCTGGTTAAAAGCGAAAGCG
AAGAAAATCGTACCGATGAACGTCTGAGCGCACTGCTGAATATGCGTCTGGATATGTATATCAAAAATCCGAGCAG
CACCGAAACCAAAGAAAATCAGAAACGTATCGGCAAGCTGAAAAAGTTCTTCAGCAACAAAATGGTGTACCTGAAA
GATAACACCCTGAGCCTGAAAAACGGCAAGAAAGAAAATATCGATCGCGAGTATAGCGAAACCGATATTCTGGAAA
GTGATGTGCGTGACAAAAAAAACTTTGCCGTCCTGAAAAAGATCTATCTGAACGAAAATGTGAACAGCGAAGAACT
GGAAGTGTTTCGCAACGACATTAAAAAGAAGCTGAACAAGATCAACAGCCTGAAATATAGCTTCGAGAAAAACAAA
GCCAACTATCAGAAGATCAACGAGAACAACATCGAAAAAGTGGAAGGTAAAAGCAAGCGCAACATCATCTATGATT
ATTATCGTGAAAGCGCCAAACGTGATGCCTATGTTAGCAATGTTAAAGAGGCCTTCGACAAGCTGTATAAAGAAGA
AGATATTGCCAAACTGGTGCTGGAAATTGAAAATCTGACCAAGCTGGAAAAATACAAGATCCGCGAATTCTATCAC
GAAATCATTGGTCGCAAAAACGATAAAGAGAACTTCGCCAAAATCATCTACGAAGAAATTCAGAACGTGAATAACA
TGAAAGAACTGATCGAGAAAGTTCCGGATATGAGCGAACTGAAAAAAAGCCAGGTGTTCTACAAATATTACCTGGA
CAAAGAGGAACTGAACGATAAAAACATCAAATACGCCTTTTGCCACTTCGTGGAAATCGAAATGAGCCAGCTGCTG
AAAAACTATGTGTATAAACGCCTGAGCAACATCAGCAACGATAAGATTAAACGCATCTTCGAGTACCAGAACCTGA
AGAAACTGATTGAAAACAAACTGCTTAACAAACTGGATACCTATGTGCGTAATTGCGGCAAATACAACTATTATCT
GCAGGATGGTGAAATTGCGACCAGCGATTTTATTGCACGTAATCGTCAGAATGAAGCCTTTCTGCGTAACATTATT
GGTGTTAGCAGCGTTGCATATTTTAGCCTGCGTAATATCCTGGAAACCGAAAACGAGAATGATATCACCGGTCGTA
TGCGTGGTAAAACCGTGAAAAACAATAAAGGCGAAGAGAAATATGTGAGCGGTGAGGTGGATAAAATCTACAACGA
AAACAAAAAGAACGAAGTGAAAGAAAACCTGAAAATGTTTTACAGCTACGACTTTAACATGGACAACAAGAACGAG
ATCGAAGATTTTTTCGCCAACATTGATGAAGCCATTAGCAGCATTCGTCATGGCATTGTTCACTTTAATCTGGAAC
TTGAGGGCAAAGACATCTTCGCGTTTAAAAACATTGCACCGAGCGAGATTAGCAAAAAGATGTTCCAGAACGAAAT
TAACGAGAAAAAACTGAAACTGAAGATCTTTCGCCAGCTGAATAGCGCAAATGTTTTTCGCTATCTTGAGAAATAC
AAAATCCTGAACTATCTGAAACGCACCCGCTTTGAATTTGTGAACAAAAACATTCCGTTTGTGCCGAGCTTTACCA
AACTGTATAGCCGTATTGATGATCTGAAAAACAGCCTGGGCATTTATTGGAAAACCCCGAAAACCAACGATGATAA
CAAGACGAAAGAAATCATCGATGCCCAGATTTATCTGCTTAAGAACATCTACTATGGCGAATTTCTGAACTATTTT
ATGAGCAACAACGGCAACTTCTTTGAAATCAGCAAAGAGATTATCGAGCTGAATAAAAACGACAAACGCAATCTGA
AAACCGGCTTCTATAAACTGCAGAAGTTTGAGGATATCCAAGAAAAGATCCCGAAAGAATATCTGGCGAATATTCA
GAGCCTGTACATGATTAATGCAGGCAATCAGGATGAGGAAGAGAAAGATACCTATATCGATTTCATCCAGAAAATC
TTTCTGAAAGGCTTTATGACCTATCTGGCCAATAATGGTCGTCTGAGTCTGATTTATATCGGTAGTGATGAAGAAA
CCAATACCAGCCTGGCAGAAAAAAAACAAGAGTTCGATAAGTTCCTGAAGAAGTACGAACAGAACAACAACATCAA
GATCCCGTATGAAATCAATGAATTTCTGCGCGAAATCAAGCTGGGCAACATTCTGAAATACACCGAACGCCTGAAT
ATGTTCTATCTGATTCTGAAACTGCTGAACCATAAAGAGCTGACGAATCTGAAAGGTAGCCTGGAAAAGTATCAGA
GCGCAAATAAAGAGGAAGCATTTAGCGATCAGCTGGAACTGATTAATCTGCTGAATCTGGATAATAACCGTGTGAC
CGAAGATTTCGAATTAGAAGCAGATGAGATCGGCAAATTCCTGGATTTTAATGGCAACAAAGTGAAGGACAACAAA
GAGCTTAAGAAGTTCGACACCAACAAGATCTATTTTGATGGCGAGAACATCATCAAACACCGTGCCTTTTATAACA
TCAAAAAATACGGTATGCTGAACCTGCTGGAAAAGATTGCAGATAAAGCAGGCTATAAAATCAGCATTGAAGAGTT
GAAAAAATACAGCAACAAGAAAAACGAGATTGAGAAAAACCACAAAATGCAAGAAAATCTGCACCGCAAATATGCA
CGTCCGCGTAAAGATGAAAAATTCACCGATGAAGATTATGAAAGCTACAAACAGGCCATCGAAAACATCGAAGAAT
ATACCCATCTGAAGAACAAAGTCGAATTCAACGAACTGAATCTGCTGCAGGGTCTGCTGCTGCGTATTCTGCATCG
TCTGGTGGGTTATACCAGCATTTGGGAACGTGATCTGCGTTTTCGCCTGAAAGGTGAATTTCCTGAAAACCAGTAT
ATCGAGGAAATCTTCAACTTCGAGAATAAAAAGAATGTGAAGTATAAAGGTGGCCAGATCGTCGAGAAATATATCA
AATTCTACAAAGAACTGCACCAGAACGACGAGGTGAAAATCAACAAATATAGCAGCGCGAACATCAAAGTGCTGAA
ACAAGAGAAAAAAGACCTGTACATCCGCAACTATATCGCCCACTTTAACTATATTCCGCATGCAGAAATTAGTCTG
CTGGAAGTTCTGGAAAACCTGCGTAAACTGCTGTCATATGATCGTAAACTTAAAAACGCCGTGATGAAAAGCGTTG
TGGACATCCTGAAAGAGTATGGTTTTGTTGCGACCTTTAAAATCGGTGCCGATAAAAAGATTGGTATTCAGACCCT
GGAAAGCGAGAAGATTGTTCACCTGAAAAATCTTAAGAAAAAGAAACTTATGACCGATCGCAATAGCGAGGAACTG
TGTAAACTGGTGAAAATTATGTTTGAGTATAAAATGGAAGAGAAGAAATCCGAAAATGGGGATCCGAATTCGAGCT
CCGTCGACAAGCTTGCGGCCGCACTCGAGCACCACCACCACCACCACTGA
LbuCas13a CTD-His Polypeptide Sequence SEQ ID NO: 2
MKVTKVGGISHKKYTSEGRLVKSESEENRTDERLSALLNMRLDMYIKNPSSTETKENQKRIGKLKKFFSNKMVYLK
DNTLSLKNGKKENIDREYSETDILESDVRDKKNFAVLKKIYLNENVNSEELEVERNDIKKKLNKINSLKYSFEKNK
ANYQKINENNIEKVEGKSKRNIIYDYYRESAKRDAYVSNVKEAFDKLYKEEDIAKLVLEIENLTKLEKYKIREFYH
EIIGRKNDKENFAKIIYEEIQNVNNMKELIEKVPDMSELKKSQVFYKYYLDKEELNDKNIKYAFCHFVEIEMSQLL
KNYVYKRLSNISNDKIKRIFEYQNLKKLIENKLLNKLDTYVRNCGKYNYYLQDGEIATSDFIARNRQNEAFLRNII
GVSSVAYFSLRNILETENENDITGRMRGKTVKNNKGEEKYVSGEVDKIYNENKKNEVKENLKMFYSYDENMDNKNE
IEDFFANIDEAISSIRHGIVHENLELEGKDIFAFKNIAPSEISKKMFQNEINEKKLKLKIFRQLNSANVFRYLEKY
KILNYLKRTRFEFVNKNIPFVPSFTKLYSRIDDLKNSLGIYWKTPKTNDDNKTKEIIDAQIYLLKNIYYGEFLNYF
MSNNGNFFEISKEIIELNKNDKRNLKTGFYKLQKFEDIQEKIPKEYLANIQSLYMINAGNQDEEEKDTYIDFIQKI
FLKGFMTYLANNGRLSLIYIGSDEETNTSLAEKKQEFDKFLKKYEQNNNIKIPYEINEFLREIKLGNILKYTERLN
MFYLILKLLNHKELTNLKGSLEKYQSANKEEAFSDQLELINLLNLDNNRVTEDFELEADEIGKFLDENGNKVKDNK
ELKKFDTNKIYFDGENIIKHRAFYNIKKYGMLNLLEKIADKAGYKISIEELKKYSNKKNEIEKNHKMQENLHRKYA
RPRKDEKFTDEDYESYKQAIENIEEYTHLKNKVEFNELNLLQGLLLRILHRLVGYTSIWERDLRFRLKGEFPENQY
IEEIFNFENKKNVKYKGGQIVEKYIKFYKELHQNDEVKINKYSSANIKVLKQEKKDLYIRNYIAHFNYIPHAEISL
LEVLENLRKLLSYDRKLKNAVMKSVVDILKEYGFVATFKIGADKKIGIQTLESEKIVHLKNLKKKKLMTDRNSEEL
CKLVKIMFEYKMEEKKSENGDPNSSSVDKLAAALEHHHHHH
LbuCas13a NTD-MBP Polynucleotide Sequence SEQ ID NO: 3
ATGAAAATCGAAGAAGGTAAACTGGTAATCTGGATTAACGGCGATAAAGGCTATAACGGTCTCGCTGAAGTCGGTA
AGAAATTCGAGAAAGATACCGGAATTAAAGTCACCGTTGAGCATCCGGATAAACTGGAAGAGAAATTCCCACAGGT
TGCGGCAACTGGCGATGGCCCTGACATTATCTTCTGGGCACACGACCGCTTTGGTGGCTACGCTCAATCTGGCCTG
TTGGCTGAAATCACCCCGGACAAAGCGTTCCAGGACAAGCTGTATCCGTTTACCTGGGATGCCGTACGTTACAACG
GCAAGCTGATTGCTTACCCGATCGCTGTTGAAGCGTTATCGCTGATTTATAACAAAGATCTGCTGCCGAACCCGCC
AAAAACCTGGGAAGAGATCCCGGCGCTGGATAAAGAACTGAAAGCGAAAGGTAAGAGCGCGCTGATGTTCAACCTG
CAAGAACCGTACTTCACCTGGCCGCTGATTGCTGCTGACGGGGGTTATGCGTTCAAGTATGAAAACGGCAAGTACG
ACATTAAAGACGTGGGCGTGGATAACGCTGGCGCGAAAGCGGGTCTGACCTTCCTGGTTGACCTGATTAAAAACAA
ACACATGAATGCAGACACCGATTACTCCATCGCAGAAGCTGCCTTTAATAAAGGCGAAACAGCGATGACCATCAAC
GGCCCGTGGGCATGGTCCAACATCGACACCAGCAAAGTGAATTATGGTGTAACGGTACTGCCGACCTTCAAGGGTC
AACCATCCAAACCGTTCGTTGGCGTGCTGAGCGCAGGTATTAACGCCGCCAGTCCGAACAAAGAGCTGGCAAAAGA
GTTCCTCGAAAACTATCTGCTGACTGATGAAGGTCTGGAAGCGGTTAATAAAGACAAACCGCTGGGTGCCGTAGCG
CTGAAGTCTTACGAGGAAGAGTTGGTGAAAGATCCGCGTATTGCCGCCACTATGGAAAACGCCCAGAAAGGTGAAA
TCATGCCGAACATCCCGCAGATGTCCGCTTTCTGGTATGCCGTGCGTACTGCGGTGATCAACGCCGCCAGCGGTCG
TCAGACTGTCGATGAAGCCCTGAAAGACGCGCAGACTAATTCGAGCTCGAACAACAACAACAATAACAATAACAAC
AACCTCGGGATCGAGGGAAGgAAGGTGACCAAAGTTGGTGGTATCAGCCATAAAAAGTATACCAGCGAAGGTCGTC
TGGTTAAAAGCGAAAGCGAAGAAAATCGTACCGATGAACGTCTGAGCGCACTGCTGAATATGCGTCTGGATATGTA
TATCAAAAATCCGAGCAGCACCGAAACCAAAGAAAATCAGAAACGTATCGGCAAGCTGAAAAAGTTCTTCAGCAAC
AAAATGGTGTACCTGAAAGATAACACCCTGAGCCTGAAAAACGGCAAGAAAGAAAATATCGATCGCGAGTATAGCG
AAACCGATATTCTGGAAAGTGATGTGCGTGACAAAAAAAACTTTGCCGTCCTGAAAAAGATCTATCTGAACGAAAA
TGTGAACAGCGAAGAACTGGAAGTGTTTCGCAACGACATTAAAAAGAAGCTGAACAAGATCAACAGCCTGAAATAT
AGCTTCGAGAAAAACAAAGCCAACTATCAGAAGATCAACGAGAACAACATCGAAAAAGTGGAAGGTAAAAGCAAGC
GCAACATCATCTATGATTATTATCGTGAAAGCGCCAAACGTGATGCCTATGTTAGCAATGTTAAAGAGGCCTTCGA
CAAGCTGTATAAAGAAGAAGATATTGCCAAACTGGTGCTGGAAATTGAAAATCTGACCAAGCTGGAAAAATACAAG
ATCCGCGAATTCTATCACGAAATCATTGGTCGCAAAAACGATAAAGAGAACTTCGCCAAAATCATCTACGAAGAAA
TTCAGAACGTGAATAACATGAAAGAACTGATCGAGAAAGTTCCGGATATGAGCGAACTGAAAAAAAGCCAGGTGTT
CTACAAATATTACCTGGACAAAGAGGAACTGAACGATAAAAACATCAAATACGCCTTTTGCCACTTCGTGGAAATC
GAAATGAGCCAGCTGCTGAAAAACTATGTGTATAAACGCCTGAGCAACATCAGCAACGATAAGATTAAACGCATCT
TCGAGTACCAGAACCTGAAGAAACTGATTGAAAACAAACTGCTTAACAAACTGGATACCTATGTGCGTAATTGCGG
CAAATACAACTATTATCTGCAGGATGGTGAAATTGCGACCAGCGATTTTATTGCACGTAATCGTCAGAATGAAGCC
TTTCTGCGTAACATTATTGGTGTTAGCAGCGTTGCATATTTTAGCCTGCGTAATATCCTGGAAACCGAAAACGAGA
ATGATATCACCGGTCGTATGCGTGGTAAAACCGTGAAAAACAATAAAGGCGAAGAGAAATATGTGAGCGGTGAGGT
GGATAAAATCTACAACGAAAACAAAAAGAACGAAGTGAAAGAAAACCTGAAAATGTTTTACAGCTACGACTTTAAC
ATGGACAACAAGAACGAGATCGAAGATTTTTTCGCCAACATTGATGAAGCCATTAGCAGCATTCGTCATGGCATTG
TTCACTTTAATCTGGAACTTGAGGGCAAAGACATCTTCGCGTTTAAAAACATTGCACCGAGCGAGATTAGCAAAAA
GATGTTCCAGAACGAAATTAACGAGAAAAAACTGAAACTGAAGATCTTTCGCCAGCTGAATAGCGCAAATGTTTTT
CGCTATCTTGAGAAATACAAAATCCTGAACTATCTGAAACGCACCCGCTTTGAATTTGTGAACAAAAACATTCCGT
TTGTGCCGAGCTTTACCAAACTGTATAGCCGTATTGATGATCTGAAAAACAGCCTGGGCATTTATTGGAAAACCCC
GAAAACCAACGATGATAACAAGACGAAAGAAATCATCGATGCCCAGATTTATCTGCTTAAGAACATCTACTATGGC
GAATTTCTGAACTATTTTATGAGCAACAACGGCAACTTCTTTGAAATCAGCAAAGAGATTATCGAGCTGAATAAAA
ACGACAAACGCAATCTGAAAACCGGCTTCTATAAACTGCAGAAGTTTGAGGATATCCAAGAAAAGATCCCGAAAGA
ATATCTGGCGAATATTCAGAGCCTGTACATGATTAATGCAGGCAATCAGGATGAGGAAGAGAAAGATACCTATATC
GATTTCATCCAGAAAATCTTTCTGAAAGGCTTTATGACCTATCTGGCCAATAATGGTCGTCTGAGTCTGATTTATA
TCGGTAGTGATGAAGAAACCAATACCAGCCTGGCAGAAAAAAAACAAGAGTTCGATAAGTTCCTGAAGAAGTACGA
ACAGAACAACAACATCAAGATCCCGTATGAAATCAATGAATTTCTGCGCGAAATCAAGCTGGGCAACATTCTGAAA
TACACCGAACGCCTGAATATGTTCTATCTGATTCTGAAACTGCTGAACCATAAAGAGCTGACGAATCTGAAAGGTA
GCCTGGAAAAGTATCAGAGCGCAAATAAAGAGGAAGCATTTAGCGATCAGCTGGAACTGATTAATCTGCTGAATCT
GGATAATAACCGTGTGACCGAAGATTTCGAATTAGAAGCAGATGAGATCGGCAAATTCCTGGATTTTAATGGCAAC
AAAGTGAAGGACAACAAAGAGCTTAAGAAGTTCGACACCAACAAGATCTATTTTGATGGCGAGAACATCATCAAAC
ACCGTGCCTTTTATAACATCAAAAAATACGGTATGCTGAACCTGCTGGAAAAGATTGCAGATAAAGCAGGCTATAA
AATCAGCATTGAAGAGTTGAAAAAATACAGCAACAAGAAAAACGAGATTGAGAAAAACCACAAAATGCAAGAAAAT
CTGCACCGCAAATATGCACGTCCGCGTAAAGATGAAAAATTCACCGATGAAGATTATGAAAGCTACAAACAGGCCA
TCGAAAACATCGAAGAATATACCCATCTGAAGAACAAAGTCGAATTCAACGAACTGAATCTGCTGCAGGGTCTGCT
GCTGCGTATTCTGCATCGTCTGGTGGGTTATACCAGCATTTGGGAACGTGATCTGCGTTTTCGCCTGAAAGGTGAA
TTTCCTGAAAACCAGTATATCGAGGAAATCTTCAACTTCGAGAATAAAAAGAATGTGAAGTATAAAGGTGGCCAGA
TCGTCGAGAAATATATCAAATTCTACAAAGAACTGCACCAGAACGACGAGGTGAAAATCAACAAATATAGCAGCGC
GAACATCAAAGTGCTGAAACAAGAGAAAAAAGACCTGTACATCCGCAACTATATCGCCCACTTTAACTATATTCCG
CATGCAGAAATTAGTCTGCTGGAAGTTCTGGAAAACCTGCGTAAACTGCTGTCATATGATCGTAAACTTAAAAACG
CCGTGATGAAAAGCGTTGTGGACATCCTGAAAGAGTATGGTTTTGTTGCGACCTTTAAAATCGGTGCCGATAAAAA
GATTGGTATTCAGACCCTGGAAAGCGAGAAGATTGTTCACCTGAAAAATCTTAAGAAAAAGAAACTTATGACCGAT
CGCAATAGCGAGGAACTGTGTAAACTGGTGAAAATTATGTTTGAGTATAAAATGGAAGAGAAGAAATCCGAAAATG
ATCCGAATTCGAGCTCCGTCGACAAGCTTGCGGCCGCACTCGAGCACCACCACCACCACCACTGA
LbuCas13a NTD-MBP Polypeptide Sequence SEQ ID NO: 4
MKIEEGKLVIWINGDKGYNGLAEVGKKFEKDTGIKVTVEHPDKLEEKFPQVAATGDGPDIIFWAHDRFGGYAQSGL
LAEITPDKAFQDKLYPFTWDAVRYNGKLIAYPIAVEALSLIYNKDLLPNPPKTWEEIPALDKELKAKGKSALMENL
QEPYFTWPLIAADGGYAFKYENGKYDIKDVGVDNAGAKAGLTFLVDLIKNKHMNADTDYSIAEAAFNKGETAMTIN
GPWAWSNIDTSKVNYGVTVLPTFKGQPSKPFVGVLSAGINAASPNKELAKEFLENYLLTDEGLEAVNKDKPLGAVA
LKSYEEELVKDPRIAATMENAQKGEIMPNIPQMSAFWYAVRTAVINAASGRQTVDEALKDAQTNSSSNNNNNNNNN
NLGIEGRKVTKVGGISHKKYTSEGRLVKSESEENRTDERLSALLNMRLDMYIKNPSSTETKENQKRIGKLKKFFSN
KMVYLKDNTLSLKNGKKENIDREYSETDILESDVRDKKNFAVLKKIYLNENVNSEELEVERNDIKKKLNKINSLKY
SFEKNKANYQKINENNIEKVEGKSKRNIIYDYYRESAKRDAYVSNVKEAFDKLYKEEDIAKLVLEIENLTKLEKYK
IREFYHEIIGRKNDKENFAKIIYEEIQNVNNMKELIEKVPDMSELKKSQVFYKYYLDKEELNDKNIKYAFCHFVEI
EMSQLLKNYVYKRLSNISNDKIKRIFEYQNLKKLIENKLLNKLDTYVRNCGKYNYYLQDGEIATSDFIARNRQNEA
FLRNIIGVSSVAYFSLRNILETENENDITGRMRGKTVKNNKGEEKYVSGEVDKIYNENKKNEVKENLKMFYSYDEN
MDNKNEIEDFFANIDEAISSIRHGIVHENLELEGKDIFAFKNIAPSEISKKMFQNEINEKKLKLKIFRQLNSANVE
RYLEKYKILNYLKRTRFEFVNKNIPFVPSFTKLYSRIDDLKNSLGIYWKTPKTNDDNKTKEIIDAQIYLLKNIYYG
EFLNYFMSNNGNFFEISKEIIELNKNDKRNLKTGFYKLQKFEDIQEKIPKEYLANIQSLYMINAGNQDEEEKDTYI
DFIQKIFLKGFMTYLANNGRLSLIYIGSDEETNTSLAEKKQEFDKFLKKYEQNNNIKIPYEINEFLREIKLGNILK
YTERLNMFYLILKLLNHKELTNLKGSLEKYQSANKEEAFSDQLELINLLNLDNNRVTEDFELEADEIGKFLDENGN
KVKDNKELKKFDTNKIYFDGENIIKHRAFYNIKKYGMLNLLEKIADKAGYKISIEELKKYSNKKNEIEKNHKMQEN
LHRKYARPRKDEKFTDEDYESYKQAIENIEEYTHLKNKVEFNELNLLQGLLLRILHRLVGYTSIWERDLRFRLKGE
FPENQYIEEIFNFENKKNVKYKGGQIVEKYIKFYKELHQNDEVKINKYSSANIKVLKQEKKDLYIRNYIAHFNYIP
HAEISLLEVLENLRKLLSYDRKLKNAVMKSVVDILKEYGFVATFKIGADKKIGIQTLESEKIVHLKNLKKKKLMTD
RNSEELCKLVKIMFEYKMEEKKSENDPNSSSVDKLAAALEHHHHHH
LshCas13a NTD-His Polynucleotide Sequence SEQ ID NO: 5
ATGGGTAACCTGTTTGGTCATAAACGTTGGTATGAAGTGCGCGACAAAAAAGACTTTAAAATCAAACGCAAGGTGA
AAGTGAAACGCAACTATGATGGCAACAAATATATCCTGAACATCAACGAGAACAACAACAAAGAGAAGATCGATAA
TAATAAATTCATCCGCAAATACATCAACTACAAAAAAAACGATAACATCCTGAAAGAATTCACCCGCAAGTTTCAT
GCAGGCAACATTCTGTTTAAACTGAAAGGCAAAGAAGGCATCATTCGCATCGAAAACAATGATGATTTTCTGGAAA
CCGAAGAGGTGGTGCTGTATATTGAAGCATATGGCAAAAGCGAAAAACTGAAGGCACTGGGCATTACCAAAAAAAA
GATTATCGATGAAGCCATTCGCCAGGGTATTACCAAAGATGACAAAAAGATCGAGATCAAGCGCCAAGAAAACGAA
GAAGAAATCGAAATTGATATCCGCGACGAGTATACCAATAAAACCCTGAATGATTGCAGCATTATTCTGCGCATTA
TCGAGAATGATGAGCTGGAAACGAAAAAGAGCATCTACGAGATCTTCAAAAACATCAACATGAGCCTGTACAAAAT
CATCGAGAAAATTATCGAAAACGAAACCGAGAAGGTGTTCGAGAATCGCTATTATGAAGAACATCTGCGTGAGAAA
CTGCTGAAAGATGATAAAATTGATGTGATCCTGACCAACTTCATGGAAATCCGCGAAAAGATTAAAAGCAACCTGG
AAATTCTGGGCTTCGTGAAATTCTATCTGAATGTTGGTGGCGACAAGAAAAAAAGCAAGAACAAGAAAATGCTGGT
CGAAAAAATTCTGAACATTAACGTTGATCTGACCGTGGAAGATATTGCCGATTTTGTGATTAAAGAGCTGGAATTC
TGGAACATCACCAAACGCATTGAGAAGGTGAAAAAAGTGAACAACGAGTTCCTGGAAAAACGTCGTAATCGCACCT
ATATCAAAAGCTATGTTCTGCTGGATAAGCACGAGAAATTCAAAATTGAACGCGAGAACAAAAAGGACAAAATCGT
GAAGTTTTTCGTGGAAAATATCAAAAACAACAGCATCAAAGAAAAAATCGAGAAGATCCTGGCCGAGTTCAAAATC
GATGAACTGATCAAAAAGCTGGAAAAAGAACTGAAAAAAGGCAACTGCGATACCGAAATTTTCGGCATCTTTAAGA
AACACTATAAAGTGAACTTCGATAGCAAAAAATTCAGCAAAAAGAGCGACGAAGAGAAAGAGCTGTATAAGATCAT
TTACCGCTATCTGAAAGGCCGTATTGAAAAAATCCTGGTGAATGAACAGAAAGTGCGCCTGAAAAAAATGGAAAAA
ATTGAGATTGAGAAGATTCTGAACGAGAGCATCCTGAGTGAGAAAATCCTGAAACGTGTTAAACAGTATACCCTGG
AACACATTATGTATCTGGGTAAACTGCGCCATAACGATATTGATATGACCACCGTTAATACCGATGATTTCAGCCG
TCTGCATGCAAAAGAAGAACTGGATCTGGAACTGATTACCTTTTTTGCAAGCACCAATATGGAACTGAACAAGATC
TTTAGCCGTGAAAACATTAACAACGACGAGAACATTGATTTCTTTGGTGGTGATCGCGAGAAAAACTATGTCCTGG
ATAAAAAGATCCTGAATAGCAAAATCAAGATCATCCGCGATCTGGATTTCATCGACAATAAGAACAACATTACCAA
CAACTTTATTCGCAAATTTACCAAAATTGGCACCAATGAACGCAACCGTATTCTGCATGCCATTAGCAAAGAACGT
GATCTGCAGGGCACCCAGGATGATTATAACAAAGTGATTAACATCATCCAGAACCTGAAAATCTCCGATGAAGAAG
TTAGCAAAGCACTGAATCTGGATGTGGTGTTCAAAGATAAGAAAAATATCATCACCAAGATCAACGATATCAAAAT
CAGCGAAGAGAACAATAACGACATCAAATATCTGCCGAGCTTTAGCAAAGTTCTGCCGGAAATTCTTAATCTGTAT
CGCAATAACCCGAAAAACGAACCGTTTGATACCATCGAAACAGAGAAAATTGTTCTGAACGCCCTGATCTATGTGA
ACAAAGAACTGTACAAGAAACTGATCCTGGAAGATGATCTGGAAGAGAACGAATCGAAAAACATCTTTCTGCAAGA
GCTGAAAAAGACCCTGGGTAACATTGATGAGATCGATGAAAACATCATCGAAAATTACTACAAGAACGCACAGATT
AGCGCAAGCAAAGGTAATAACAAAGCCATCAAAAAATACCAGAAAAAGGTGATCGAATGCTACATTGGTTATCTGC
GCAAAAACTACGAAGAACTGTTCGATTTCAGCGATTTCAAAATGAACATCCAAGAGATCAAGAAGCAGATCAAGGA
CATTAACGACAACAAAACCTATGAACGCATCACCGTTAAAACCAGCGATAAAACCATTGTGATCAACGACGATTTC
GAGTACATCATTAGCATTTTTGCACTGCTGAATTCCAACGCCGTGATCAACAAAATTCGCAATCGCTTTTTTGCCA
CCAGTGTTTGGCTGAATACCAGCGAATATCAGAACATTATCGATATCCTGGATGAGATCATGCAGCTGAATACACT
GCGTAATGAATGCATTACCGAAAACTGGAATCTGAACCTTGAAGAATTTATTCAGAAAATGAAAGAGATCGAGAAA
GACTTCGACGACTTCAAAATCCAGACCAAAAAAGAAATCTTCAACAACTACTACGAGGACATCAAAAATAACATTC
TGACCGAATTCAAAGACGATATTAACGGCTGTGACGTGCTGGAAAAGAAGTTGGAAAAGATCGTTATCTTCGATGA
CGAAACCAAATTCGAAATCGACAAAAAGTCCAACATCCTTCAGGATGAACAGCGTAAACTGAGCAATATCAACAAG
AAAGACCTGAAGAAGAAGGTCGACCAGTACATCAAAGACAAAGACCAAGAAATTAAGAGCAAAATCCTGTGCCGCA
TCATCTTTAACAGCGACTTTCTGAAAAAGTATAAGAAAGAGATTGACAACCTGATCGAGGATATGGAAAGCGAGAA
CGAAAACAAGTTTCAAGAGATCTACTATCCGAAAGAACGCAAAAACGAGCTGTACATCTACAAGAAGAACCTGTTC
CTGAATATTGGCAACCCGAACTTCGACAAAATCTATGGTCTGATCAGCAACGACATTAAAATGGCCGATGCAAAAT
TCCTGTTTAATATCGATGGTAAAAACATCCGTAAAAACAAAATTAGCGAGATCGACGCGATCCTGAAAAACCTGAA
CGATAAACTGAATGGCTACAGCAAAGAATATAAAGAGAAATACATTAAAAAGCTGAAAGAAAATGACGACTTCTTC
GCCAAGAACATCCAGAATAAAAACTATAAAAGCTTCGAGAAGGACTACAATCGCGTGTCCGAATATAAGAAAATTC
GTGATCTGGTGGAATTCAACTATCTGAACAAAATCGAAAGCTATCTGATCGATATCAACTGGAAACTGGCAATTCA
GATGGCACGTTTTGAGCGTGATATGCACTATATTGTTAATGGTCTGCGTGAACTGGGCATCATTAAACTGAGTGGT
TATAATACCGGCATTAGCCGTGCATATCCGAAACGTAATGGTTCCGATGGTTTTTATACCACCACCGCCTATTACA
AATTTTTCGACGAAGAAAGCTACAAGAAATTTGAGAAAATTTGCTACGGCTTCGGCATTGATCTGAGCGAAAATAG
CGAAATTAACAAGCCGGAAAATGAGAGCATTCGCAACTATATCTCCCACTTTTATATCGTGCGTAATCCGTTTGCC
GATTATAGCATTGCAGAGCAGATTGATCGTGTTAGCAATCTGCTGAGCTATAGTACCCGTTATAACAATAGCACCT
ATGCCAGCGTGTTTGAGGTGTTTAAAAAGGATGTTAACCTGGACTATGACGAGCTGAAGAAAAAGTTCAAACTGAT
CGGCAACAATGACATCCTGGAACGTCTGATGAAACCGAAAAAAGTTAGTGTGCTGGAACTTGAGAGCTACAACAGC
GATTATATCAAGAACCTGATTATCGAGCTGCTGACCAAGATTGAAAATACCAATGATACCCTGGGGGATCCGAATT
CGAGCTCCGTCGACAAGCTTGCGGCCGCACTCGAGCACCACCACCACCACCACTGA
LshCas13a NTD-His Polypeptide Sequence SEQ ID NO: 6
MGNLFGHKRWYEVRDKKDFKIKRKVKVKRNYDGNKYILNINENNNKEKIDNNKFIRKYINYKKNDNILKEFTRKFH
AGNILFKLKGKEGIIRIENNDDFLETEEVVLYIEAYGKSEKLKALGITKKKIIDEAIRQGITKDDKKIEIKRQENE
EEIEIDIRDEYTNKTLNDCSIILRIIENDELETKKSIYEIFKNINMSLYKIIEKIIENETEKVFENRYYEEHLREK
LLKDDKIDVILTNFMEIREKIKSNLEILGFVKFYLNVGGDKKKSKNKKMLVEKILNINVDLTVEDIADFVIKELEF
WNITKRIEKVKKVNNEFLEKRRNRTYIKSYVLLDKHEKFKIERENKKDKIVKFFVENIKNNSIKEKIEKILAEFKI
DELIKKLEKELKKGNCDTEIFGIFKKHYKVNFDSKKESKKSDEEKELYKIIYRYLKGRIEKILVNEQKVRLKKMEK
IEIEKILNESILSEKILKRVKQYTLEHIMYLGKLRHNDIDMTTVNTDDFSRLHAKEELDLELITFFASTNMELNKI
FSRENINNDENIDFFGGDREKNYVLDKKILNSKIKIIRDLDFIDNKNNITNNFIRKFTKIGTNERNRILHAISKER
DLQGTQDDYNKVINIIQNLKISDEEVSKALNLDVVFKDKKNIITKINDIKISEENNNDIKYLPSFSKVLPEILNLY
RNNPKNEPFDTIETEKIVLNALIYVNKELYKKLILEDDLEENESKNIFLQELKKTLGNIDEIDENIIENYYKNAQI
SASKGNNKAIKKYQKKVIECYIGYLRKNYEELFDFSDFKMNIQEIKKQIKDINDNKTYERITVKTSDKTIVINDDF
EYIISIFALLNSNAVINKIRNRFFATSVWLNTSEYQNIIDILDEIMQLNTLRNECITENWNLNLEEFIQKMKEIEK
DFDDFKIQTKKEIFNNYYEDIKNNILTEFKDDINGCDVLEKKLEKIVIFDDETKFEIDKKSNILQDEQRKLSNINK
KDLKKKVDQYIKDKDQEIKSKILCRIIFNSDFLKKYKKEIDNLIEDMESENENKFQEIYYPKERKNELYIYKKNLF
LNIGNPNFDKIYGLISNDIKMADAKFLFNIDGKNIRKNKISEIDAILKNLNDKLNGYSKEYKEKYIKKLKENDDFF
AKNIQNKNYKSFEKDYNRVSEYKKIRDLVEFNYLNKIESYLIDINWKLAIQMARFERDMHYIVNGLRELGIIKLSG
YNTGISRAYPKRNGSDGFYTTTAYYKFFDEESYKKFEKICYGFGIDLSENSEINKPENESIRNYISHFYIVRNPFA
DYSIAEQIDRVSNLLSYSTRYNNSTYASVFEVFKKDVNLDYDELKKKFKLIGNNDILERLMKPKKVSVLELESYNS
DYIKNLIIELLTKIENTNDTLGDPNSSSVDKLAAALEHHHHHH
LshCas13a NTD-MBP Polynucleotide Sequence SEQ ID NO: 7
ATGAAAATCGAAGAAGGTAAACTGGTAATCTGGATTAACGGCGATAAAGGCTATAACGGTCTCGCTGAAGTCGGTA
AGAAATTCGAGAAAGATACCGGAATTAAAGTCACCGTTGAGCATCCGGATAAACTGGAAGAGAAATTCCCACAGGT
TGCGGCAACTGGCGATGGCCCTGACATTATCTTCTGGGCACACGACCGCTTTGGTGGCTACGCTCAATCTGGCCTG
TTGGCTGAAATCACCCCGGACAAAGCGTTCCAGGACAAGCTGTATCCGTTTACCTGGGATGCCGTACGTTACAACG
GCAAGCTGATTGCTTACCCGATCGCTGTTGAAGCGTTATCGCTGATTTATAACAAAGATCTGCTGCCGAACCCGCC
AAAAACCTGGGAAGAGATCCCGGCGCTGGATAAAGAACTGAAAGCGAAAGGTAAGAGCGCGCTGATGTTCAACCTG
CAAGAACCGTACTTCACCTGGCCGCTGATTGCTGCTGACGGGGGTTATGCGTTCAAGTATGAAAACGGCAAGTACG
ACATTAAAGACGTGGGCGTGGATAACGCTGGCGCGAAAGCGGGTCTGACCTTCCTGGTTGACCTGATTAAAAACAA
ACACATGAATGCAGACACCGATTACTCCATCGCAGAAGCTGCCTTTAATAAAGGCGAAACAGCGATGACCATCAAC
GGCCCGTGGGCATGGTCCAACATCGACACCAGCAAAGTGAATTATGGTGTAACGGTACTGCCGACCTTCAAGGGTC
AACCATCCAAACCGTTCGTTGGCGTGCTGAGCGCAGGTATTAACGCCGCCAGTCCGAACAAAGAGCTGGCAAAAGA
GTTCCTCGAAAACTATCTGCTGACTGATGAAGGTCTGGAAGCGGTTAATAAAGACAAACCGCTGGGTGCCGTAGCG
CTGAAGTCTTACGAGGAAGAGTTGGTGAAAGATCCGCGTATTGCCGCCACTATGGAAAACGCCCAGAAAGGTGAAA
TCATGCCGAACATCCCGCAGATGTCCGCTTTCTGGTATGCCGTGCGTACTGCGGTGATCAACGCCGCCAGCGGTCG
TCAGACTGTCGATGAAGCCCTGAAAGACGCGCAGACTAATTCGAGCTCGAACAACAACAACAATAACAATAACAAC
AACCTCGGGATCGAGGGAAGGGGTAACCTGTTTGGTCATAAACGTTGGTATGAAGTGCGCGACAAAAAAGACTTTA
AAATCAAACGCAAGGTGAAAGTGAAACGCAACTATGATGGCAACAAATATATCCTGAACATCAACGAGAACAACAA
CAAAGAGAAGATCGATAATAATAAATTCATCCGCAAATACATCAACTACAAAAAAAACGATAACATCCTGAAAGAA
TTCACCCGCAAGTTTCATGCAGGCAACATTCTGTTTAAACTGAAAGGCAAAGAAGGCATCATTCGCATCGAAAACA
ATGATGATTTTCTGGAAACCGAAGAGGTGGTGCTGTATATTGAAGCATATGGCAAAAGCGAAAAACTGAAGGCACT
GGGCATTACCAAAAAAAAGATTATCGATGAAGCCATTCGCCAGGGTATTACCAAAGATGACAAAAAGATCGAGATC
AAGCGCCAAGAAAACGAAGAAGAAATCGAAATTGATATCCGCGACGAGTATACCAATAAAACCCTGAATGATTGCA
GCATTATTCTGCGCATTATCGAGAATGATGAGCTGGAAACGAAAAAGAGCATCTACGAGATCTTCAAAAACATCAA
CATGAGCCTGTACAAAATCATCGAGAAAATTATCGAAAACGAAACCGAGAAGGTGTTCGAGAATCGCTATTATGAA
GAACATCTGCGTGAGAAACTGCTGAAAGATGATAAAATTGATGTGATCCTGACCAACTTCATGGAAATCCGCGAAA
AGATTAAAAGCAACCTGGAAATTCTGGGCTTCGTGAAATTCTATCTGAATGTTGGTGGCGACAAGAAAAAAAGCAA
GAACAAGAAAATGCTGGTCGAAAAAATTCTGAACATTAACGTTGATCTGACCGTGGAAGATATTGCCGATTTTGTG
ATTAAAGAGCTGGAATTCTGGAACATCACCAAACGCATTGAGAAGGTGAAAAAAGTGAACAACGAGTTCCTGGAAA
AACGTCGTAATCGCACCTATATCAAAAGCTATGTTCTGCTGGATAAGCACGAGAAATTCAAAATTGAACGCGAGAA
CAAAAAGGACAAAATCGTGAAGTTTTTCGTGGAAAATATCAAAAACAACAGCATCAAAGAAAAAATCGAGAAGATC
CTGGCCGAGTTCAAAATCGATGAACTGATCAAAAAGCTGGAAAAAGAACTGAAAAAAGGCAACTGCGATACCGAAA
TTTTCGGCATCTTTAAGAAACACTATAAAGTGAACTTCGATAGCAAAAAATTCAGCAAAAAGAGCGACGAAGAGAA
AGAGCTGTATAAGATCATTTACCGCTATCTGAAAGGCCGTATTGAAAAAATCCTGGTGAATGAACAGAAAGTGCGC
CTGAAAAAAATGGAAAAAATTGAGATTGAGAAGATTCTGAACGAGAGCATCCTGAGTGAGAAAATCCTGAAACGTG
TTAAACAGTATACCCTGGAACACATTATGTATCTGGGTAAACTGCGCCATAACGATATTGATATGACCACCGTTAA
TACCGATGATTTCAGCCGTCTGCATGCAAAAGAAGAACTGGATCTGGAACTGATTACCTTTTTTGCAAGCACCAAT
ATGGAACTGAACAAGATCTTTAGCCGTGAAAACATTAACAACGACGAGAACATTGATTTCTTTGGTGGTGATCGCG
AGAAAAACTATGTCCTGGATAAAAAGATCCTGAATAGCAAAATCAAGATCATCCGCGATCTGGATTTCATCGACAA
TAAGAACAACATTACCAACAACTTTATTCGCAAATTTACCAAAATTGGCACCAATGAACGCAACCGTATTCTGCAT
GCCATTAGCAAAGAACGTGATCTGCAGGGCACCCAGGATGATTATAACAAAGTGATTAACATCATCCAGAACCTGA
AAATCTCCGATGAAGAAGTTAGCAAAGCACTGAATCTGGATGTGGTGTTCAAAGATAAGAAAAATATCATCACCAA
GATCAACGATATCAAAATCAGCGAAGAGAACAATAACGACATCAAATATCTGCCGAGCTTTAGCAAAGTTCTGCCG
GAAATTCTTAATCTGTATCGCAATAACCCGAAAAACGAACCGTTTGATACCATCGAAACAGAGAAAATTGTTCTGA
ACGCCCTGATCTATGTGAACAAAGAACTGTACAAGAAACTGATCCTGGAAGATGATCTGGAAGAGAACGAATCGAA
AAACATCTTTCTGCAAGAGCTGAAAAAGACCCTGGGTAACATTGATGAGATCGATGAAAACATCATCGAAAATTAC
TACAAGAACGCACAGATTAGCGCAAGCAAAGGTAATAACAAAGCCATCAAAAAATACCAGAAAAAGGTGATCGAAT
GCTACATTGGTTATCTGCGCAAAAACTACGAAGAACTGTTCGATTTCAGCGATTTCAAAATGAACATCCAAGAGAT
CAAGAAGCAGATCAAGGACATTAACGACAACAAAACCTATGAACGCATCACCGTTAAAACCAGCGATAAAACCATT
GTGATCAACGACGATTTCGAGTACATCATTAGCATTTTTGCACTGCTGAATTCCAACGCCGTGATCAACAAAATTC
GCAATCGCTTTTTTGCCACCAGTGTTTGGCTGAATACCAGCGAATATCAGAACATTATCGATATCCTGGATGAGAT
CATGCAGCTGAATACACTGCGTAATGAATGCATTACCGAAAACTGGAATCTGAACCTTGAAGAATTTATTCAGAAA
ATGAAAGAGATCGAGAAAGACTTCGACGACTTCAAAATCCAGACCAAAAAAGAAATCTTCAACAACTACTACGAGG
ACATCAAAAATAACATTCTGACCGAATTCAAAGACGATATTAACGGCTGTGACGTGCTGGAAAAGAAGTTGGAAAA
GATCGTTATCTTCGATGACGAAACCAAATTCGAAATCGACAAAAAGTCCAACATCCTTCAGGATGAACAGCGTAAA
CTGAGCAATATCAACAAGAAAGACCTGAAGAAGAAGGTCGACCAGTACATCAAAGACAAAGACCAAGAAATTAAGA
GCAAAATCCTGTGCCGCATCATCTTTAACAGCGACTTTCTGAAAAAGTATAAGAAAGAGATTGACAACCTGATCGA
GGATATGGAAAGCGAGAACGAAAACAAGTTTCAAGAGATCTACTATCCGAAAGAACGCAAAAACGAGCTGTACATC
TACAAGAAGAACCTGTTCCTGAATATTGGCAACCCGAACTTCGACAAAATCTATGGTCTGATCAGCAACGACATTA
AAATGGCCGATGCAAAATTCCTGTTTAATATCGATGGTAAAAACATCCGTAAAAACAAAATTAGCGAGATCGACGC
GATCCTGAAAAACCTGAACGATAAACTGAATGGCTACAGCAAAGAATATAAAGAGAAATACATTAAAAAGCTGAAA
GAAAATGACGACTTCTTCGCCAAGAACATCCAGAATAAAAACTATAAAAGCTTCGAGAAGGACTACAATCGCGTGT
CCGAATATAAGAAAATTCGTGATCTGGTGGAATTCAACTATCTGAACAAAATCGAAAGCTATCTGATCGATATCAA
CTGGAAACTGGCAATTCAGATGGCACGTTTTGAGCGTGATATGCACTATATTGTTAATGGTCTGCGTGAACTGGGC
ATCATTAAACTGAGTGGTTATAATACCGGCATTAGCCGTGCATATCCGAAACGTAATGGTTCCGATGGTTTTTATA
CCACCACCGCCTATTACAAATTTTTCGACGAAGAAAGCTACAAGAAATTTGAGAAAATTTGCTACGGCTTCGGCAT
TGATCTGAGCGAAAATAGCGAAATTAACAAGCCGGAAAATGAGAGCATTCGCAACTATATCTCCCACTTTTATATC
GTGCGTAATCCGTTTGCCGATTATAGCATTGCAGAGCAGATTGATCGTGTTAGCAATCTGCTGAGCTATAGTACCC
GTTATAACAATAGCACCTATGCCAGCGTGTTTGAGGTGTTTAAAAAGGATGTTAACCTGGACTATGACGAGCTGAA
GAAAAAGTTCAAACTGATCGGCAACAATGACATCCTGGAACGTCTGATGAAACCGAAAAAAGTTAGTGTGCTGGAA
CTTGAGAGCTACAACAGCGATTATATCAAGAACCTGATTATCGAGCTGCTGACCAAGATTGAAAATACCAATGATA
CCCTGGATCCGAATTCGAGCTCCGTCGACAAGCTTGCGGCCGCACTCGAGCACCACCACCACCACCACTGA
LshCas13a NTD-MBP Polypeptide Sequence SEQ ID NO: 8
MKIEEGKLVIWINGDKGYNGLAEVGKKFEKDTGIKVTVEHPDKLEEKFPQVAATGDGPDIIFWAHDRFGGYAQSGL
LAEITPDKAFQDKLYPFTWDAVRYNGKLIAYPIAVEALSLIYNKDLLPNPPKTWEEIPALDKELKAKGKSALMENL
QEPYFTWPLIAADGGYAFKYENGKYDIKDVGVDNAGAKAGLTFLVDLIKNKHMNADTDYSIAEAAFNKGETAMTIN
GPWAWSNIDTSKVNYGVTVLPTFKGQPSKPFVGVLSAGINAASPNKELAKEFLENYLLTDEGLEAVNKDKPLGAVA
LKSYEEELVKDPRIAATMENAQKGEIMPNIPQMSAFWYAVRTAVINAASGRQTVDEALKDAQTNSSSNNNNNNNNN
NLGIEGRGNLFGHKRWYEVRDKKDFKIKRKVKVKRNYDGNKYILNINENNNKEKIDNNKFIRKYINYKKNDNILKE
FTRKFHAGNILFKLKGKEGIIRIENNDDFLETEEVVLYIEAYGKSEKLKALGITKKKIIDEAIRQGITKDDKKIEI
KRQENEEEIEIDIRDEYTNKTLNDCSIILRIIENDELETKKSIYEIFKNINMSLYKIIEKIIENETEKVFENRYYE
EHLREKLLKDDKIDVILTNFMEIREKIKSNLEILGFVKFYLNVGGDKKKSKNKKMLVEKILNINVDLTVEDIADFV
IKELEFWNITKRIEKVKKVNNEFLEKRRNRTYIKSYVLLDKHEKFKIERENKKDKIVKFFVENIKNNSIKEKIEKI
LAEFKIDELIKKLEKELKKGNCDTEIFGIFKKHYKVNFDSKKFSKKSDEEKELYKIIYRYLKGRIEKILVNEQKVR
LKKMEKIEIEKILNESILSEKILKRVKQYTLEHIMYLGKLRHNDIDMTTVNTDDFSRLHAKEELDLELITFFASTN
MELNKIFSRENINNDENIDFFGGDREKNYVLDKKILNSKIKIIRDLDFIDNKNNITNNFIRKFTKIGTNERNRILH
AISKERDLQGTQDDYNKVINIIQNLKISDEEVSKALNLDVVFKDKKNIITKINDIKISEENNNDIKYLPSFSKVLP
EILNLYRNNPKNEPFDTIETEKIVLNALIYVNKELYKKLILEDDLEENESKNIFLQELKKTLGNIDEIDENIIENY
YKNAQISASKGNNKAIKKYQKKVIECYIGYLRKNYEELFDFSDFKMNIQEIKKQIKDINDNKTYERITVKTSDKTI
VINDDFEYIISIFALLNSNAVINKIRNRFFATSVWLNTSEYQNIIDILDEIMQLNTLRNECITENWNLNLEEFIQK
MKEIEKDFDDFKIQTKKEIFNNYYEDIKNNILTEFKDDINGCDVLEKKLEKIVIFDDETKFEIDKKSNILQDEQRK
LSNINKKDLKKKVDQYIKDKDQEIKSKILCRIIFNSDFLKKYKKEIDNLIEDMESENENKFQEIYYPKERKNELYI
YKKNLFLNIGNPNEDKIYGLISNDIKMADAKFLFNIDGKNIRKNKISEIDAILKNLNDKLNGYSKEYKEKYIKKLK
ENDDFFAKNIQNKNYKSFEKDYNRVSEYKKIRDLVEFNYLNKIESYLIDINWKLAIQMARFERDMHYIVNGLRELG
IIKLSGYNTGISRAYPKRNGSDGFYTTTAYYKFFDEESYKKFEKICYGFGIDLSENSEINKPENESIRNYISHFYI
VRNPFADYSIAEQIDRVSNLLSYSTRYNNSTYASVFEVFKKDVNLDYDELKKKFKLIGNNDILERLMKPKKVSVLE
LESYNSDYIKNLIIELLTKIENTNDTLDPNSSSVDKLAAALEHHHHHH
LwaCas13a CTD-His Polynucleotide Sequence SEQ ID NO: 9
ATGAAAGTGACCAAAGTGGATGGCATCAGCCACAAAAAATACATCGAAGAAGGCAAACTGGTTAAAAGCACCAGCG
AAGAAAATCGTACCAGCGAACGTCTGAGCGAACTGCTGAGCATTCGTCTGGATATCTATATCAAAAATCCGGATAA
TGCCAGCGAGGAAGAAAACCGTATTCGTCGTGAAAACCTGAAAAAGTTCTTCAGCAATAAAGTGCTGCACCTGAAA
GATAGCGTTCTGTATCTGAAAAACCGCAAAGAAAAAAATGCCGTGCAGGACAAAAACTATAGCGAAGAGGATATCA
GCGAGTATGACCTGAAGAACAAAAATAGCTTTAGCGTGCTGAAAAAAATCCTGCTGAATGAAGATGTGAATAGCGA
GGAACTGGAAATCTTTCGTAAAGATGTTGAAGCCAAGCTGAACAAAATCAACAGCCTGAAATATAGCTTTGAAGAA
AACAAGGCCAACTATCAGAAAATCAACGAGAACAACGTGGAAAAAGTTGGTGGTAAAAGCAAACGCAACATCATCT
ATGATTATTATCGCGAAAGCGCGAAACGCAACGATTATATCAATAATGTGCAAGAGGCCTTCGACAAACTGTACAA
AAAAGAGGACATCGAAAAACTGTTTTTTCTGATCGAGAACAGCAAGAAGCACGAGAAATACAAAATCCGCGAGTAC
TACCATAAAATCATCGGTCGCAAAAACGATAAAGAGAACTTCGCCAAAATCATCTACGAAGAAATTCAGAACGTGA
ACAACATCAAAGAACTGATCGAAAAAATTCCGGACATGAGCGAGCTGAAGAAAAGCCAGGTGTTCTATAAATACTA
CCTGGACAAAGAGGAACTGAACGACAAAAACATCAAATATGCCTTTTGCCACTTCGTCGAAATTGAAATGAGCCAG
CTGCTTAAAAACTACGTGTATAAACGCCTGAGCAACATCAGCAACGATAAAATCAAACGTATCTTTGAATATCAGA
ATCTGAAGAAACTGATTGAAAACAAACTGCTGAACAAGCTGGATACCTATGTTCGTAATTGCGGCAAATACAACTA
CTATCTGCAGGTTGGTGAAATTGCAACCAGCGATTTTATTGCACGTAATCGTCAGAATGAAGCCTTTCTGCGTAAC
ATTATTGGTGTTAGCAGCGTTGCATATTTTAGCCTGCGTAATATTCTGGAAACCGAAAACGAAAATGGCATTACCG
GTCGTATGCGTGGTAAAACCGTTAAAAACAATAAAGGCGAAGAGAAGTATGTGAGCGGTGAAGTGGATAAAATCTA
TAACGAAAACAAGCAGAACGAAGTGAAAGAAAATCTGAAAATGTTTTACAGCTACGACTTCAACATGGACAACAAA
AACGAGATCGAAGATTTCTTCGCCAACATTGATGAAGCCATTAGCAGTATTCGTCATGGCATTGTGCACTTTAATC
TGGAACTTGAAGGCAAAGACATCTTCGCGTTTAAAAACATTGCACCGAGCGAGATCAGCAAAAAAATGTTTCAGAA
CGAGATTAACGAAAAAAAACTGAAACTGAAAATCTTCAAACAGCTGAATAGCGCCAACGTGTTCAACTATTATGAG
AAAGACGTGATCATCAAATACCTTAAAAACACCAAATTCAACTTCGTGAATAAAAACATCCCGTTTGTTCCGAGCT
TCACCAAACTGTATAACAAAATTGAAGATCTGCGCAATACCCTGAAGTTTTTTTGGAGCGTTCCGAAAGACAAAGA
AGAAAAAGACGCACAGATCTACCTGCTTAAGAACATCTATTATGGCGAATTTCTGAACAAATTCGTGAAAAATAGC
AAAGTGTTCTTCAAAATCACCAACGAGGTGATCAAGATTAACAAACAGCGTAATCAGAAAACCGGTCACTACAAAT
ACCAGAAGTTTGAGAACATTGAAAAAACCGTGCCGGTTGAATATCTGGCAATTATTCAGAGCCGTGAGATGATTAA
CAACCAGGATAAAGAAGAGAAAAACACCTACATCGATTTCATCCAGCAGATCTTTCTGAAAGGCTTTATCGATTAC
CTGAACAAGAACAACCTGAAGTATATCGAGTCGAACAACAATAACGACAACAACGACATCTTTAGCAAAATCAAAA
TCAAGAAAGATAATAAAGAAAAATACGACAAGATCCTGAAAAACTATGAGAAGCACAACCGCAACAAAGAAATTCC
GCATGAGATCAATGAATTTGTGCGCGAAATTAAACTGGGCAAAATCCTGAAATACACCGAGAACCTGAATATGTTC
TATCTGATTCTGAAGCTGCTGAACCATAAAGAGCTGACCAATCTGAAAGGTAGCCTGGAAAAATATCAGAGCGCAA
ACAAAGAAGAGACATTTTCTGACGAACTGGAACTGATTAATCTGCTGAATCTGGATAATAACCGTGTGACCGAAGA
TTTTGAACTGGAAGCAAATGAAATCGGCAAATTCCTGGATTTCAATGAGAACAAAATTAAGGACCGGAAAGAGCTT
AAAAAGTTTGATACCAACAAAATCTACTTCGACGGCGAGAACATTATCAAACATCGTGCCTTTTATAACATCAAAA
AGTATGGCATGCTGAACCTGCTGGAAAAAATTGCAGATAAAGCCAAGTACAAAATTAGCCTGAAAGAACTTAAAGA
GTACAGCAACAAAAAGAACGAAATCGAGAAGAACTATACCATGCAGCAGAATCTGCATCGTAAATATGCACGTCCG
AAAAAAGACGAGAAATTCAACGATGAGGACTATAAAGAATACGAGAAAGCCATTGGCAACATCCAGAAATATACCC
ACTTGAAAAACAAAGTGGAATTTAACGAGCTGAATTTACTGCAGGGTCTGCTGCTGAAAATTCTGCACCGTCTGGT
TGGTTATACCAGCATTTGGGAACGTGATCTGCGTTTTCGCCTGAAAGGTGAATTTCCTGAAAACCACTATATCGAG
GAAATTTTCAACTTTGACAACAGCAAAAACGTGAAATATAAGAGCGGTCAGATCGTCGAAAAGTACATCAACTTTT
ACAAAGAACTTTACAAGGATAATGTGGAAAAACGCAGCATCTACAGCGACAAGAAAGTGAAAAAGCTGAAGCAAGA
AAAGAAAGACCTGTACATCCGTAATTATATCGCCCACTTTAACTATATCCCGCATGCAGAAATTAGTCTGCTGGAA
GTTCTGGAAAATCTGCGTAAACTGCTGTCATATGATCGCAAACTGAAGAACGCAATCATGAAAAGCATTGTGGATA
TCCTGAAAGAGTATGGTTTTGTCGCCACCTTTAAAATCGGTGCCGATAAGAAAATTGAGATTCAGACCCTGGAAAG
CGAGAAAATTGTGCATCTTAAGAACCTTAAAAAGAAAAAACTGATGACCGATCGCAACAGCGAAGAGTTATGTGAA
CTGGTGAAAGTGATGTTCGAATACAAAGCACTGGAAGGGGATCCGAATTCGAGCTCCGTCGACAAGCTTGCGGCCG
CACTCGAGCACCACCACCACCACCACTGA
LwaCas13a CTD-His Polypeptide Sequence SEQ ID NO: 10
MKVTKVDGISHKKYIEEGKLVKSTSEENRTSERLSELLSIRLDIYIKNPDNASEEENRIRRENLKKFFSNKVLHLK
DSVLYLKNRKEKNAVQDKNYSEEDISEYDLKNKNSFSVLKKILLNEDVNSEELEIFRKDVEAKLNKINSLKYSFEE
NKANYQKINENNVEKVGGKSKRNIIYDYYRESAKRNDYINNVQEAFDKLYKKEDIEKLFFLIENSKKHEKYKIREY
YHKIIGRKNDKENFAKIIYEEIQNVNNIKELIEKIPDMSELKKSQVFYKYYLDKEELNDKNIKYAFCHFVEIEMSQ
LLKNYVYKRLSNISNDKIKRIFEYQNLKKLIENKLLNKLDTYVRNCGKYNYYLQVGEIATSDFIARNRQNEAFLRN
IIGVSSVAYFSLRNILETENENGITGRMRGKTVKNNKGEEKYVSGEVDKIYNENKQNEVKENLKMFYSYDENMDNK
NEIEDFFANIDEAISSIRHGIVHENLELEGKDIFAFKNIAPSEISKKMFQNEINEKKLKLKIFKQLNSANVENYYE
KDVIIKYLKNTKFNFVNKNIPFVPSFTKLYNKIEDLRNTLKFFWSVPKDKEEKDAQIYLLKNIYYGEFLNKFVKNS
KVFFKITNEVIKINKQRNQKTGHYKYQKFENIEKTVPVEYLAIIQSREMINNQDKEEKNTYIDFIQQIFLKGFIDY
LNKNNLKYIESNNNNDNNDIFSKIKIKKDNKEKYDKILKNYEKHNRNKEIPHEINEFVREIKLGKILKYTENLNME
YLILKLLNHKELTNLKGSLEKYQSANKEETFSDELELINLLNLDNNRVTEDFELEANEIGKFLDENENKIKDRKEL
KKFDTNKIYFDGENIIKHRAFYNIKKYGMLNLLEKIADKAKYKISLKELKEYSNKKNEIEKNYTMQQNLHRKYARP
KKDEKFNDEDYKEYEKAIGNIQKYTHLKNKVEFNELNLLQGLLLKILHRLVGYTSIWERDLRFRLKGEFPENHYIE
EIFNFDNSKNVKYKSGQIVEKYINFYKELYKDNVEKRSIYSDKKVKKLKQEKKDLYIRNYIAHENYIPHAEISLLE
VLENLRKLLSYDRKLKNAIMKSIVDILKEYGFVATFKIGADKKIEIQTLESEKIVHLKNLKKKKLMTDRNSEELCE
LVKVMFEYKALEGDPNSSSVDKLAAALEHHHHHH
LwaCas13a NTD-MBP Polynucleotide Sequence SEQ ID NO: 11
ATGAAAATCGAAGAAGGTAAACTGGTAATCTGGATTAACGGCGATAAAGGCTATAACGGTCTCGCTGAAGTCGGTA
AGAAATTCGAGAAAGATACCGGAATTAAAGTCACCGTTGAGCATCCGGATAAACTGGAAGAGAAATTCCCACAGGT
TGCGGCAACTGGCGATGGCCCTGACATTATCTTCTGGGCACACGACCGCTTTGGTGGCTACGCTCAATCTGGCCTG
TTGGCTGAAATCACCCCGGACAAAGCGTTCCAGGACAAGCTGTATCCGTTTACCTGGGATGCCGTACGTTACAACG
GCAAGCTGATTGCTTACCCGATCGCTGTTGAAGCGTTATCGCTGATTTATAACAAAGATCTGCTGCCGAACCCGCC
AAAAACCTGGGAAGAGATCCCGGCGCTGGATAAAGAACTGAAAGCGAAAGGTAAGAGCGCGCTGATGTTCAACCTG
CAAGAACCGTACTTCACCTGGCCGCTGATTGCTGCTGACGGGGGTTATGCGTTCAAGTATGAAAACGGCAAGTACG
ACATTAAAGACGTGGGCGTGGATAACGCTGGCGCGAAAGCGGGTCTGACCTTCCTGGTTGACCTGATTAAAAACAA
ACACATGAATGCAGACACCGATTACTCCATCGCAGAAGCTGCCTTTAATAAAGGCGAAACAGCGATGACCATCAAC
GGCCCGTGGGCATGGTCCAACATCGACACCAGCAAAGTGAATTATGGTGTAACGGTACTGCCGACCTTCAAGGGTC
AACCATCCAAACCGTTCGTTGGCGTGCTGAGCGCAGGTATTAACGCCGCCAGTCCGAACAAAGAGCTGGCAAAAGA
GTTCCTCGAAAACTATCTGCTGACTGATGAAGGTCTGGAAGCGGTTAATAAAGACAAACCGCTGGGTGCCGTAGCG
CTGAAGTCTTACGAGGAAGAGTTGGTGAAAGATCCGCGTATTGCCGCCACTATGGAAAACGCCCAGAAAGGTGAAA
TCATGCCGAACATCCCGCAGATGTCCGCTTTCTGGTATGCCGTGCGTACTGCGGTGATCAACGCCGCCAGCGGTCG
TCAGACTGTCGATGAAGCCCTGAAAGACGCGCAGACTAATTCGAGCTCGAACAACAACAACAATAACAATAACAAC
AACCTCGGGATCGAGGGAAGgAAAGTGACCAAAGTGGATGGCATCAGCCACAAAAAATACATCGAAGAAGGCAAAC
TGGTTAAAAGCACCAGCGAAGAAAATCGTACCAGCGAACGTCTGAGCGAACTGCTGAGCATTCGTCTGGATATCTA
TATCAAAAATCCGGATAATGCCAGCGAGGAAGAAAACCGTATTCGTCGTGAAAACCTGAAAAAGTTCTTCAGCAAT
AAAGTGCTGCACCTGAAAGATAGCGTTCTGTATCTGAAAAACCGCAAAGAAAAAAATGCCGTGCAGGACAAAAACT
ATAGCGAAGAGGATATCAGCGAGTATGACCTGAAGAACAAAAATAGCTTTAGCGTGCTGAAAAAAATCCTGCTGAA
TGAAGATGTGAATAGCGAGGAACTGGAAATCTTTCGTAAAGATGTTGAAGCCAAGCTGAACAAAATCAACAGCCTG
AAATATAGCTTTGAAGAAAACAAGGCCAACTATCAGAAAATCAACGAGAACAACGTGGAAAAAGTTGGTGGTAAAA
GCAAACGCAACATCATCTATGATTATTATCGCGAAAGCGCGAAACGCAACGATTATATCAATAATGTGCAAGAGGC
CTTCGACAAACTGTACAAAAAAGAGGACATCGAAAAACTGTTTTTTCTGATCGAGAACAGCAAGAAGCACGAGAAA
TACAAAATCCGCGAGTACTACCATAAAATCATCGGTCGCAAAAACGATAAAGAGAACTTCGCCAAAATCATCTACG
AAGAAATTCAGAACGTGAACAACATCAAAGAACTGATCGAAAAAATTCCGGACATGAGCGAGCTGAAGAAAAGCCA
GGTGTTCTATAAATACTACCTGGACAAAGAGGAACTGAACGACAAAAACATCAAATATGCCTTTTGCCACTTCGTC
GAAATTGAAATGAGCCAGCTGCTTAAAAACTACGTGTATAAACGCCTGAGCAACATCAGCAACGATAAAATCAAAC
GTATCTTTGAATATCAGAATCTGAAGAAACTGATTGAAAACAAACTGCTGAACAAGCTGGATACCTATGTTCGTAA
TTGCGGCAAATACAACTACTATCTGCAGGTTGGTGAAATTGCAACCAGCGATTTTATTGCACGTAATCGTCAGAAT
GAAGCCTTTCTGCGTAACATTATTGGTGTTAGCAGCGTTGCATATTTTAGCCTGCGTAATATTCTGGAAACCGAAA
ACGAAAATGGTATTACCGGTCGTATGCGTGGTAAAACCGTTAAAAACAATAAAGGCGAAGAGAAGTATGTGAGCGG
TGAAGTGGATAAAATCTATAACGAAAACAAGCAGAACGAAGTGAAAGAAAATCTGAAAATGTTTTACAGCTACGAC
TTCAACATGGACAACAAAAACGAGATCGAAGATTTCTTCGCCAACATTGATGAAGCCATTAGCAGTATTCGTCATG
GCATTGTGCACTTTAATCTGGAACTTGAAGGCAAAGACATCTTCGCGTTTAAAAACATTGCACCGAGCGAGATCAG
CAAAAAAATGTTTCAGAACGAGATTAACGAAAAAAAACTGAAACTGAAAATCTTCAAACAGCTGAATAGCGCCAAC
GTGTTCAACTATTATGAGAAAGACGTGATCATCAAATACCTTAAAAACACCAAATTCAACTTCGTGAATAAAAACA
TCCCGTTTGTTCCGAGCTTCACCAAACTGTATAACAAAATTGAAGATCTGCGCAATACCCTGAAGTTTTTTTGGAG
CGTTCCGAAAGACAAAGAAGAAAAAGACGCACAGATCTACCTGCTTAAGAACATCTATTATGGCGAATTTCTGAAC
AAATTCGTGAAAAATAGCAAAGTGTTCTTCAAAATCACCAACGAGGTGATCAAGATTAACAAACAGCGTAATCAGA
AAACCGGTCACTACAAATACCAGAAGTTTGAGAACATTGAAAAAACCGTGCCGGTTGAATATCTGGCAATTATTCA
GAGCCGTGAGATGATTAACAACCAGGATAAAGAAGAGAAAAACACCTACATCGATTTCATCCAGCAGATCTTTCTG
AAAGGCTTTATCGATTACCTGAACAAGAACAACCTGAAGTATATCGAGTCGAACAACAATAACGACAACAACGACA
TCTTTAGCAAAATCAAAATCAAGAAAGATAATAAAGAAAAATACGACAAGATCCTGAAAAACTATGAGAAGCACAA
CCGCAACAAAGAAATTCCGCATGAGATCAATGAATTTGTGCGCGAAATTAAACTGGGCAAAATCCTGAAATACACC
GAGAACCTGAATATGTTCTATCTGATTCTGAAGCTGCTGAACCATAAAGAGCTGACCAATCTGAAAGGTAGCCTGG
AAAAATATCAGAGCGCAAACAAAGAAGAGACATTTTCTGACGAACTGGAACTGATTAATCTGCTGAATCTGGATAA
TAACCGTGTGACCGAAGATTTTGAACTGGAAGCAAATGAAATCGGCAAATTCCTGGATTTCAATGAGAACAAAATT
AAGGACCGGAAAGAGCTTAAAAAGTTTGATACCAACAAAATCTACTTCGACGGCGAGAACATTATCAAACATCGTG
CCTTTTATAACATCAAAAAGTATGGCATGCTGAACCTGCTGGAAAAAATTGCAGATAAAGCCAAGTACAAAATTAG
CCTGAAAGAACTTAAAGAGTACAGCAACAAAAAGAACGAAATCGAGAAGAACTATACCATGCAGCAGAATCTGCAT
CGTAAATATGCACGTCCGAAAAAAGACGAGAAATTCAACGATGAGGACTATAAAGAATACGAGAAAGCCATTGGCA
ACATCCAGAAATATACCCACTTGAAAAACAAAGTGGAATTTAACGAGCTGAATTTACTGCAGGGTCTGCTGCTGAA
AATTCTGCACCGTCTGGTTGGTTATACCAGCATTTGGGAACGTGATCTGCGTTTTCGCCTGAAAGGTGAATTTCCT
GAAAACCACTATATCGAGGAAATTTTCAACTTTGACAACAGCAAAAACGTGAAATATAAGAGCGGTCAGATCGTCG
AAAAGTACATCAACTTTTACAAAGAACTTTACAAGGATAATGTGGAAAAACGCAGCATCTACAGCGACAAGAAAGT
GAAAAAGCTGAAGCAAGAAAAGAAAGACCTGTACATCCGTAATTATATCGCCCACTTTAACTATATCCCGCATGCA
GAAATTAGTCTGCTGGAAGTTCTGGAAAATCTGCGTAAACTGCTGTCATATGATCGCAAACTGAAGAACGCAATCA
TGAAAAGCATTGTGGATATCCTGAAAGAGTATGGTTTTGTCGCCACCTTTAAAATCGGTGCCGATAAGAAAATTGA
GATTCAGACCCTGGAAAGCGAGAAAATTGTGCATCTTAAGAACCTTAAAAAGAAAAAACTGATGACCGATCGCAAC
AGCGAAGAGTTATGTGAACTGGTGAAAGTGATGTTCGAATACAAAGCACTGGAAGATCCGAATTCGAGCTCCGTCG
ACAAGCTTGCGGCCGCACTCGAGCACCACCACCACCACCACTGA
LwaCas13a NTD-MBP Polypeptide Sequence SEQ ID NO: 12
MKIEEGKLVIWINGDKGYNGLAEVGKKFEKDTGIKVTVEHPDKLEEKFPQVAATGDGPDIIFWAHDRFGGYAQSGL
LAEITPDKAFQDKLYPFTWDAVRYNGKLIAYPIAVEALSLIYNKDLLPNPPKTWEEIPALDKELKAKGKSALMENL
QEPYFTWPLIAADGGYAFKYENGKYDIKDVGVDNAGAKAGLTFLVDLIKNKHMNADTDYSIAEAAFNKGETAMTIN
GPWAWSNIDTSKVNYGVTVLPTFKGQPSKPFVGVLSAGINAASPNKELAKEFLENYLLTDEGLEAVNKDKPLGAVA
LKSYEEELVKDPRIAATMENAQKGEIMPNIPQMSAFWYAVRTAVINAASGRQTVDEALKDAQTNSSSNNNNNNNNN
NLGIEGRKVTKVDGISHKKYIEEGKLVKSTSEENRTSERLSELLSIRLDIYIKNPDNASEEENRIRRENLKKFFSN
KVLHLKDSVLYLKNRKEKNAVQDKNYSEEDISEYDLKNKNSFSVLKKILLNEDVNSEELEIFRKDVEAKLNKINSL
KYSFEENKANYQKINENNVEKVGGKSKRNIIYDYYRESAKRNDYINNVQEAFDKLYKKEDIEKLFFLIENSKKHEK
YKIREYYHKIIGRKNDKENFAKIIYEEIQNVNNIKELIEKIPDMSELKKSQVFYKYYLDKEELNDKNIKYAFCHFV
EIEMSQLLKNYVYKRLSNISNDKIKRIFEYQNLKKLIENKLLNKLDTYVRNCGKYNYYLQVGEIATSDFIARNRQN
EAFLRNIIGVSSVAYFSLRNILETENENGITGRMRGKTVKNNKGEEKYVSGEVDKIYNENKQNEVKENLKMFYSYD
FNMDNKNEIEDFFANIDEAISSIRHGIVHFNLELEGKDIFAFKNIAPSEISKKMFQNEINEKKLKLKIFKQLNSAN
VFNYYEKDVIIKYLKNTKFNFVNKNIPFVPSFTKLYNKIEDLRNTLKFFWSVPKDKEEKDAQIYLLKNIYYGEFLN
KFVKNSKVFFKITNEVIKINKQRNQKTGHYKYQKFENIEKTVPVEYLAIIQSREMINNQDKEEKNTYIDFIQQIFL
KGFIDYLNKNNLKYIESNNNNDNNDIFSKIKIKKDNKEKYDKILKNYEKHNRNKEIPHEINEFVREIKLGKILKYT
ENLNMFYLILKLLNHKELTNLKGSLEKYQSANKEETFSDELELINLLNLDNNRVTEDFELEANEIGKFLDENENKI
KDRKELKKFDTNKIYFDGENIIKHRAFYNIKKYGMLNLLEKIADKAKYKISLKELKEYSNKKNEIEKNYTMQQNLH
RKYARPKKDEKFNDEDYKEYEKAIGNIQKYTHLKNKVEFNELNLLQGLLLKILHRLVGYTSIWERDLRFRLKGEFP
ENHYIEEIFNFDNSKNVKYKSGQIVEKYINFYKELYKDNVEKRSIYSDKKVKKLKQEKKDLYIRNYIAHFNYIPHA
EISLLEVLENLRKLLSYDRKLKNAIMKSIVDILKEYGFVATFKIGADKKIEIQTLESEKIVHLKNLKKKKLMTDRN
SEELCELVKVMFEYKALEDPNSSSVDKLAAALEHHHHHH
LwaCas13a G403D CTD-His Polynucleotide Sequence SEQ ID NO: 13
ATGAAAGTGACCAAAGTGGATGGCATCAGCCACAAAAAATACATCGAAGAAGGCAAACTGGTTAAAAGCACCAGCG
AAGAAAATCGTACCAGCGAACGTCTGAGCGAACTGCTGAGCATTCGTCTGGATATCTATATCAAAAATCCGGATAA
TGCCAGCGAGGAAGAAAACCGTATTCGTCGTGAAAACCTGAAAAAGTTCTTCAGCAATAAAGTGCTGCACCTGAAA
GATAGCGTTCTGTATCTGAAAAACCGCAAAGAAAAAAATGCCGTGCAGGACAAAAACTATAGCGAAGAGGATATCA
GCGAGTATGACCTGAAGAACAAAAATAGCTTTAGCGTGCTGAAAAAAATCCTGCTGAATGAAGATGTGAATAGCGA
GGAACTGGAAATCTTTCGTAAAGATGTTGAAGCCAAGCTGAACAAAATCAACAGCCTGAAATATAGCTTTGAAGAA
AACAAGGCCAACTATCAGAAAATCAACGAGAACAACGTGGAAAAAGTTGGTGGTAAAAGCAAACGCAACATCATCT
ATGATTATTATCGCGAAAGCGCGAAACGCAACGATTATATCAATAATGTGCAAGAGGCCTTCGACAAACTGTACAA
AAAAGAGGACATCGAAAAACTGTTTTTTCTGATCGAGAACAGCAAGAAGCACGAGAAATACAAAATCCGCGAGTAC
TACCATAAAATCATCGGTCGCAAAAACGATAAAGAGAACTTCGCCAAAATCATCTACGAAGAAATTCAGAACGTGA
ACAACATCAAAGAACTGATCGAAAAAATTCCGGACATGAGCGAGCTGAAGAAAAGCCAGGTGTTCTATAAATACTA
CCTGGACAAAGAGGAACTGAACGACAAAAACATCAAATATGCCTTTTGCCACTTCGTCGAAATTGAAATGAGCCAG
CTGCTTAAAAACTACGTGTATAAACGCCTGAGCAACATCAGCAACGATAAAATCAAACGTATCTTTGAATATCAGA
ATCTGAAGAAACTGATTGAAAACAAACTGCTGAACAAGCTGGATACCTATGTTCGTAATTGCGGCAAATACAACTA
CTATCTGCAGGTTGGTGAAATTGCAACCAGCGATTTTATTGCACGTAATCGTCAGAATGAAGCCTTTCTGCGTAAC
ATTATTGGTGTTAGCAGCGTTGCATATTTTAGCCTGCGTAATATTCTGGAAACCGAAAACGAAAATGATATTACCG
GTCGTATGCGTGGTAAAACCGTTAAAAACAATAAAGGCGAAGAGAAGTATGTGAGCGGTGAAGTGGATAAAATCTA
TAACGAAAACAAGCAGAACGAAGTGAAAGAAAATCTGAAAATGTTTTACAGCTACGACTTCAACATGGACAACAAA
AACGAGATCGAAGATTTCTTCGCCAACATTGATGAAGCCATTAGCAGTATTCGTCATGGCATTGTGCACTTTAATC
TGGAACTTGAAGGCAAAGACATCTTCGCGTTTAAAAACATTGCACCGAGCGAGATCAGCAAAAAAATGTTTCAGAA
CGAGATTAACGAAAAAAAACTGAAACTGAAAATCTTCAAACAGCTGAATAGCGCCAACGTGTTCAACTATTATGAG
AAAGACGTGATCATCAAATACCTTAAAAACACCAAATTCAACTTCGTGAATAAAAACATCCCGTTTGTTCCGAGCT
TCACCAAACTGTATAACAAAATTGAAGATCTGCGCAATACCCTGAAGTTTTTTTGGAGCGTTCCGAAAGACAAAGA
AGAAAAAGACGCACAGATCTACCTGCTTAAGAACATCTATTATGGCGAATTTCTGAACAAATTCGTGAAAAATAGC
AAAGTGTTCTTCAAAATCACCAACGAGGTGATCAAGATTAACAAACAGCGTAATCAGAAAACCGGTCACTACAAAT
ACCAGAAGTTTGAGAACATTGAAAAAACCGTGCCGGTTGAATATCTGGCAATTATTCAGAGCCGTGAGATGATTAA
CAACCAGGATAAAGAAGAGAAAAACACCTACATCGATTTCATCCAGCAGATCTTTCTGAAAGGCTTTATCGATTAC
CTGAACAAGAACAACCTGAAGTATATCGAGTCGAACAACAATAACGACAACAACGACATCTTTAGCAAAATCAAAA
TCAAGAAAGATAATAAAGAAAAATACGACAAGATCCTGAAAAACTATGAGAAGCACAACCGCAACAAAGAAATTCC
GCATGAGATCAATGAATTTGTGCGCGAAATTAAACTGGGCAAAATCCTGAAATACACCGAGAACCTGAATATGTTC
TATCTGATTCTGAAGCTGCTGAACCATAAAGAGCTGACCAATCTGAAAGGTAGCCTGGAAAAATATCAGAGCGCAA
ACAAAGAAGAGACATTTTCTGACGAACTGGAACTGATTAATCTGCTGAATCTGGATAATAACCGTGTGACCGAAGA
TTTTGAACTGGAAGCAAATGAAATCGGCAAATTCCTGGATTTCAATGAGAACAAAATTAAGGACCGGAAAGAGCTT
AAAAAGTTTGATACCAACAAAATCTACTTCGACGGCGAGAACATTATCAAACATCGTGCCTTTTATAACATCAAAA
AGTATGGCATGCTGAACCTGCTGGAAAAAATTGCAGATAAAGCCAAGTACAAAATTAGCCTGAAAGAACTTAAAGA
GTACAGCAACAAAAAGAACGAAATCGAGAAGAACTATACCATGCAGCAGAATCTGCATCGTAAATATGCACGTCCG
AAAAAAGACGAGAAATTCAACGATGAGGACTATAAAGAATACGAGAAAGCCATTGGCAACATCCAGAAATATACCC
ACTTGAAAAACAAAGTGGAATTTAACGAGCTGAATTTACTGCAGGGTCTGCTGCTGAAAATTCTGCACCGTCTGGT
TGGTTATACCAGCATTTGGGAACGTGATCTGCGTTTTCGCCTGAAAGGTGAATTTCCTGAAAACCACTATATCGAG
GAAATTTTCAACTTTGACAACAGCAAAAACGTGAAATATAAGAGCGGTCAGATCGTCGAAAAGTACATCAACTTTT
ACAAAGAACTTTACAAGGATAATGTGGAAAAACGCAGCATCTACAGCGACAAGAAAGTGAAAAAGCTGAAGCAAGA
AAAGAAAGACCTGTACATCCGTAATTATATCGCCCACTTTAACTATATCCCGCATGCAGAAATTAGTCTGCTGGAA
GTTCTGGAAAATCTGCGTAAACTGCTGTCATATGATCGCAAACTGAAGAACGCAATCATGAAAAGCATTGTGGATA
TCCTGAAAGAGTATGGTTTTGTCGCCACCTTTAAAATCGGTGCCGATAAGAAAATTGAGATTCAGACCCTGGAAAG
CGAGAAAATTGTGCATCTTAAGAACCTTAAAAAGAAAAAACTGATGACCGATCGCAACAGCGAAGAGTTATGTGAA
CTGGTGAAAGTGATGTTCGAATACAAAGCACTGGAAGGGGATCCGAATTCGAGCTCCGTCGACAAGCTTGCGGCCG
CACTCGAGCACCACCACCACCACCACTGA
LwaCas13a G403D CTD-His Polypeptide Sequence SEQ ID NO: 14
MKVTKVDGISHKKYIEEGKLVKSTSEENRTSERLSELLSIRLDIYIKNPDNASEEENRIRRENLKKFFSNKVLHLK
DSVLYLKNRKEKNAVQDKNYSEEDISEYDLKNKNSFSVLKKILLNEDVNSEELEIFRKDVEAKLNKINSLKYSFEE
NKANYQKINENNVEKVGGKSKRNIIYDYYRESAKRNDYINNVQEAFDKLYKKEDIEKLFFLIENSKKHEKYKIREY
YHKIIGRKNDKENFAKIIYEEIQNVNNIKELIEKIPDMSELKKSQVFYKYYLDKEELNDKNIKYAFCHFVEIEMSQ
LLKNYVYKRLSNISNDKIKRIFEYQNLKKLIENKLLNKLDTYVRNCGKYNYYLQVGEIATSDFIARNRQNEAFLRN
IIGVSSVAYFSLRNILETENENDITGRMRGKTVKNNKGEEKYVSGEVDKIYNENKQNEVKENLKMFYSYDENMDNK
NEIEDFFANIDEAISSIRHGIVHFNLELEGKDIFAFKNIAPSEISKKMFQNEINEKKLKLKIFKQLNSANVENYYE
KDVIIKYLKNTKFNFVNKNIPFVPSFTKLYNKIEDLRNTLKFFWSVPKDKEEKDAQIYLLKNIYYGEFLNKFVKNS
KVFFKITNEVIKINKQRNQKTGHYKYQKFENIEKTVPVEYLAIIQSREMINNQDKEEKNTYIDFIQQIFLKGFIDY
LNKNNLKYIESNNNNDNNDIFSKIKIKKDNKEKYDKILKNYEKHNRNKEIPHEINEFVREIKLGKILKYTENLNMF
YLILKLLNHKELTNLKGSLEKYQSANKEETFSDELELINLLNLDNNRVTEDFELEANEIGKFLDENENKIKDRKEL
KKFDTNKIYFDGENIIKHRAFYNIKKYGMLNLLEKIADKAKYKISLKELKEYSNKKNEIEKNYTMQQNLHRKYARP
KKDEKFNDEDYKEYEKAIGNIQKYTHLKNKVEFNELNLLQGLLLKILHRLVGYTSIWERDLRFRLKGEFPENHYIE
EIFNFDNSKNVKYKSGQIVEKYINFYKELYKDNVEKRSIYSDKKVKKLKQEKKDLYIRNYIAHFNYIPHAEISLLE
VLENLRKLLSYDRKLKNAIMKSIVDILKEYGFVATFKIGADKKIEIQTLESEKIVHLKNLKKKKLMTDRNSEELCE
LVKVMFEYKALEGDPNSSSVDKLAAALEHHHHHH
LbuCas 13a-CTD-His Vector (pET28b) SEQ ID NO: 36
LbuCas13a-NTD-MBP Vector (pET28b-MBP-TEV) SEQ ID NO: 37
LshCas13a-NTD-His Vector (pET28b) SEQ ID NO: 38
LshCas13a-NTD-MBP Vector (pET28b-MBP-TEV) SEQ ID NO: 39
LwaCas 13a-CTD-His Vector (pET28b) SEQ ID NO: 40
LwaCas 13a-NTD-MBP Vector (pET28b-MBP-TEV) SEQ ID NO: 41
LwaCas13a G403D-CTD-His Vector (pET28b) SEQ ID NO: 42
pET28b SEQ ID NO: 43
pET28-MBP-TEV SEQ ID NO: 44

TABLE 2
Sequences of primers used for isothermal assembly (ISO).

| Primer Name | Sequence (5′→3′) | SEQ ID NO |
|---|---|---|
| Lbu 5′ for pET28 ISO | GAAATAATTTTGTTTAACTTTAAGAAGGAGATATACC | SEQ ID NO: 15 |
| | ATGAAGGTGACCAAAGTTGGTGG | |
| Lbu 3′ for pET28 ISO | CGGCCGCAAGCTTGTCGACGGAGCTCGAATTCGGATC | SEQ ID NO: 16 |
| | CCCATTTTCGGATTTCTTCTCTTCCATTTTATACTC | |
| Lbu 5′ for pMAL ISO | AATAACAATAACAACAACCTCGGGATCGAGGGAAGGA | SEQ ID NO: 17 |
| | AGGTGACCAAAGTTGGTGGTATC | |
| Lbu 3′ for pMAL ISO | GTGCGGCCGCAAGCTTGTCGACGGAGCTCGAATTCGG | SEQ ID NO: 18 |
| | ATCATTTTCGGATTTCTTCTCTTCCATTTTATACTC | |
| Lsh 5′ for pET28 ISO | ATAATTTTGTTTAACTTTAAGAAGGAGATATACCATG | SEQ ID NO: 19 |
| | GGTAACCTGTTTGGTCATAAACG | |
| Lsh 3′ for pET28 ISO | CGGCCGCAAGCTTGTCGACGGAGCTCGAATTCGGATC | SEQ ID NO: 20 |
| | CCCCAGGGTATCATTGGTATTTTCAATCTTGG | |
| Lsh 5′ for pMAL ISO | TAACAATAACAACAACCTCGGGATCGAGGGAAGGGGT | SEQ ID NO: 21 |
| | AACCTGTTTGGTCATAAACGTTG | |
| Lsh 3′ for pMAL ISO | GTGCGGCCGCAAGCTTGTCGACGGAGCTCGAATTCGG | SEQ ID NO: 22 |
| | ATCCAGGGTATCATTGGTATTTTCAATCTTGG | |
| Lwa 5′ for pET28 ISO | AAATAATTTTGTTTAACTTTAAGAAGGAGATATACCA | SEQ ID NO: 23 |
| | TGAAAGTGACCAAAGTGGATGG | |
| Lwa 3′ for pET28 ISO | GCAAGCTTGTCGACGGAGCTCGAATTCGGATCCCCTT | SEQ ID NO: 24 |
| | CCAGTGCTTTGTATTCGAACATC | |
| Lwa 5′ for pMAL ISO | ACAATAACAATAACAACAACCTCGGGATCGAGGGAAG | SEQ ID NO: 25 |
| | GAAAGTGACCAAAGTGGATGGCA | |
| Lwa 3′ for pMAL ISO | CAAGCTTGTCGACGGAGCTCGAATTCGGATCCCCTTC | SEQ ID NO: 26 |
| | CAGTGCTTTGTATTCGAACATCA | |
| pET28 3′ Fwd for ISO | GGGGATCCGAATTCGAGCTC | SEQ ID NO: 27 |
| pET28 5′ Rev for ISO | GGTATATCTCCTTCTTAAAGTTAAACAAAATTATTTC | SEQ ID NO: 28 |
| pMAL 3′ Fwd for ISO | GATCCGAATTCGAGCTCCGT | SEQ ID NO: 29 |
| pMAL 5′ Rev for ISO | CCTTCCCTCGATCCCGAGG | SEQ ID NO: 30 |
| LwaCas13a G403D | GTAATATTCTGGAAACCGAAAACGAAAATGATATTAC | SEQ ID NO: 31 |
| Fwd | CGGTCGTATGCGTGGT | |
| LwaCas13a G403D | ACCACGCATACGACCGGTAATATCATTTTCGTTTTCG | SEQ ID NO: 32 |
| Rev | GTTTCCAGAATATTAC | |

After transformation into E. coli cells, plasmid DNA was isolated and sequenced to verify the desired sequence. The resulting plasmids were transformed into E. coli BL21(DE3) cells for protein expression.

A colony with the appropriate strain was used to inoculate TB media (1 L) with kanamycin (0.05 mg/mL) and grown at 37° C. until an OD₆₀₀ of approximately 0.6 was reached, then the flask was cooled to 18° C. for 30 minutes. The addition of 1 M IPTG (500 μL) was used to induce protein expression, followed by growth at 18° C. for 19 hours. Cells were harvested at 4700×g for 10 minutes at 4° C.

The cell pellet was re-suspended in a lysis buffer containing the following: 20 mM NaPO₄ pH 6.8, 0.5 M NaCl, 10 mM imidazole, 5% glycerol, DNase I, 10 mM CaCl₂), lysozyme (1 mg/mL), protease inhibitor and 1% CHAPS. The cells were lysed using an Avestin Emulsiflex C3 homogenizer pre-chilled to 4° C. at 15-20 kpsi with three passes. The lysate was centrifuged at 16,000×g for 20 minutes at 4° C. to remove cell debris.

The cleared lysate for 6× histidine tagged Cas13 proteins was loaded on a HisTrap™ HP column (Cytiva). The procedure consisted of equilibrating the resin with His·Bind® buffer (20 mM NaPO₄ PH 6.8, 0.5 M NaCl, 10 mM imidazole, 5% glycerol), followed by sample loading. The column was washed with His·Bind® buffer, followed by a 0.5% Triton-X114 wash, followed by an additional standard wash and a 10% wash consisting of 10% His-Elution buffer (10 mM NaPO₄ pH 6.8, 500 mM NaCl, 150 mM imidazole, 5% glycerol). Finally, the sample was eluted using His-Elution buffer.

Alternatively, Cas13a variants from the pET28-MBP-TEV expression plasmid were loaded on MBPTrap™ HP column (Cytiva). The procedure consisted of equilibrating the resin with MBP-Bind buffer (20 mM Tris. HCl PH 7.4, 500 mM NaCl, 1 mM EDTA, 10% glycerol), followed by sample loading. The sample was then washed with MBP-Bind buffer. The sample was eluted using MBP-Elution buffer (20 mM Tris·HCl PH 7.4, 500 mM NaCl, 1 mM EDTA, 10 mM maltose, 10% glycerol).

The partially purified Cas13a variants were then loaded on a HiTrap™ SP strong cation exchange column (Cytiva). The procedure consisted of equilibrating the resin with SP-Bind buffer (20 mM Tris·HCl pH 8.0, 130 mM NaCl, 1 mM DTT, 5% glycerol), followed by sample loading. The sample was then washed with SP-Bind buffer. The sample was eluted using a linear gradient to 50% SP-Elution buffer (20 mM Tris·HCl PH 8.0, 2 M NaCl, 1 mM DTT, 5% glycerol). The Cas13a variants eluted from the column at a NaCl concentration between 0.4-0.5 M.

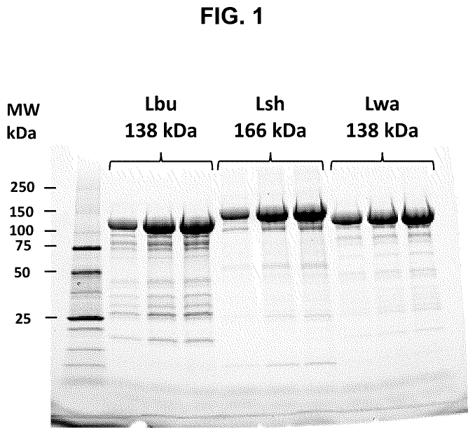

The purified Cas13a variants were concentrated to approximately 10 mg/ml using an Amicon® Ultra-15 (Sigma Aldrich) with a 10 K MWCO filter by centrifuging at 4000×g. The concentrated protein was placed in a hydrated Slide-A-Lyzer™ dialysis cassette (Thermo Fisher) with a 10K MWCO and dialyzed against three rounds of dialysis buffer (50 mM Tris. HCl PH 7.5, 0.6 M NaCl, 2 mM DTT, 50% glycerol). The final concentration was determined by a Nano Drop 8000 (Thermo Scientific) and stored at −20° C. (see FIG. 1 for SDS-PAGE).

### Example 2

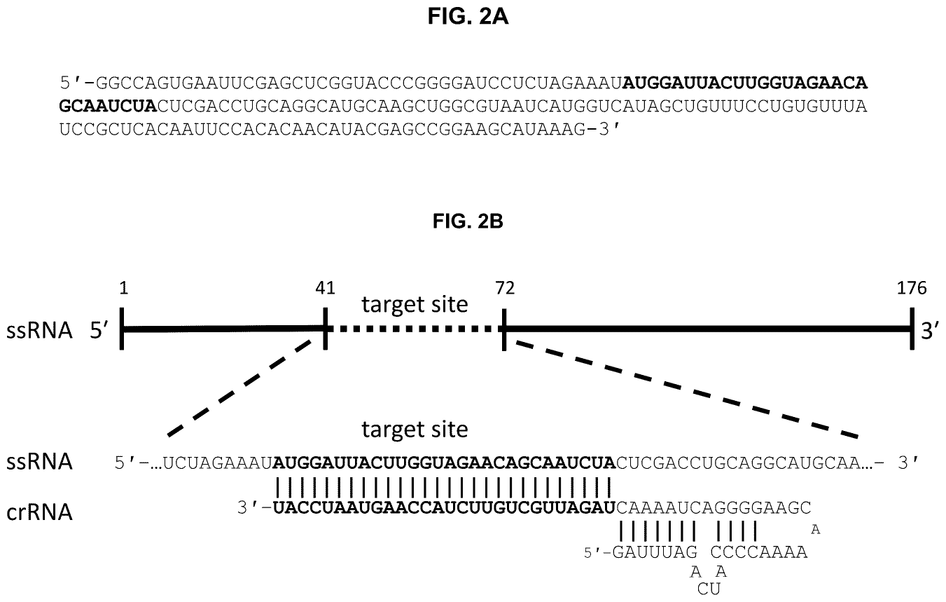

The activity of Cas13a proteins were assayed by observing the non-specific RNase activity in the degradation of fluorescent-labeled RNA. The nucleic acid target (FIG. 2A) was first ordered as two Ultramer® DNA Oligos (Integrated DNA Technologies) and annealed together by heating at 95° C. for 5 min in duplex buffer with a slow cool to room temperature. The dsDNA target was transcribed to RNA by the HiScribe™ T7 High Yield RNA Synthesis Kit (New England Biolabs), followed by a clean-up with the MEGAclear™ Purification Kit (Applied Biosystems). The RNP complex (FIG. 2B) was formed by combining purified Cas13a protein and the corresponding crRNA (Table 3) and incubating at room temperature for 10 minutes.

TABLE 3
Sequences of crRNA for each Cas13a protein variant

| Cas13a | | SEQ ID |
|---|---|---|
| variant | Ribonucleotide Sequences (5′→3′) | NO |
| LwaCas13a | GGGGAUUUAGACUACCCCAAAAACGAAGGGGACUAAAACUAGAUUGCUGU | 33 |
| | UCUACCAAGUAAUCCAU | |
| LbuCas13a | GACCACCCCAAAAAUGAAGGGGACUAAAACAUAGAUUGCUGUUCUACCAA | 34 |
| | GUAAUCCAU | |
| LshCas13a | CCACCCCAAUAUCGAAGGGGACUAAAACUAGAUUGCUGUUCUACCAAGUA | 35 |
| | AUCCAU | |

All nucleotides are ribonucleotides; spacer sequences are underlined.

The Cas13a RNP complex (1 μM) was titrated down with nuclease reaction buffer (40 mM Tris·HCl PH 7.4, 60 mM NaCl, 6 mM MgCl₂) in two-fold dilutions to 1 nM RNP to afford a wide range of Cas13a nuclease reactions. The activity of the Cas13a RNP complex was measured by the addition of RNA reporter (degradation reporter probe) (200 nM, RNaseAlert™ Substrate), RNase inhibitor (1 μL, SUPERase-In™), total human RNA (25 ng, purified from HEK-293 cells), RNA target (20 ng) in nuclease reaction buffer (total volume of 100 μL). Reactions were allowed to proceed for 10 min at 37° C., followed by detection on a fluorescent plate reader (TECAN) using the fluorescein channel (490 nm excitation, 520 nm emission).

These results show a rapid visualization of nucleic acid degradation with LbuCas13a using only 4 nM RNP (Table 4). These proteins were purified using a C-terminal 6× histidine tag.

TABLE 4
Cas13a RNP activity assay data after 10 min at 37° C.

| Lbu | | Lsh | | Lwa | | Lwa G403D | |
|---|---|---|---|---|---|---|---|
| RNP | Emis- | RNP | Emis- | RNP | Emis- | RNP | Emis- |
| (nM) | sion | (nM) | sion | (nM) | sion | (nM) | sion |
| 1000 | 17182 | 1000 | 1165 | 1000 | 2948 | 1000 | 8989 |
| 500 | 31575 | 500 | 880 | 500 | 2387 | 500 | 12706 |
| 250 | 41002 | 250 | 739 | 250 | 2113 | 250 | 19003 |
| 125 | 39324 | 125 | 629 | 125 | 1740 | 125 | 22328 |
| 63 | 38526 | 63 | 581 | 63 | 1418 | 63 | 18684 |
| 31 | 50516 | 31 | 568 | 31 | 1158 | 31 | 6429 |
| 16 | 51035 | 16 | 560 | 16 | 1081 | 16 | 1058 |
| 8 | 49605 | 8 | 535 | 8 | 872 | 8 | 738 |
| 4 | 50376 | 4 | 550 | 4 | 752 | 4 | 693 |
| 2 | 25572 | 2 | 552 | 2 | 612 | 2 | 678 |
| 1 | 22674 | 1 | 528 | 1 | 581 | 1 | 676 |
| 0 | 1371 | 0 | 901 | 0 | 1886 | 0 | 5039 |

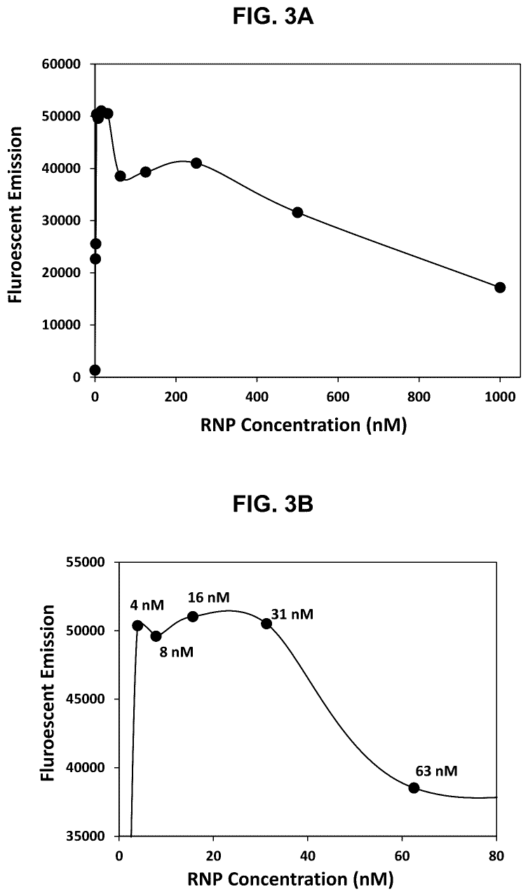

While Lwa and Lsh Cas13a were described in the literature as potentially useful Cas13 variants, RNase activity of these variants was not observed at the concentration ranges for RNP complex used in this study (Table 4). For LbuCas13a, there was a clear bell-like curve representation of the data (FIG. 3A-B). As the concentration of RNP soared from 31 nM to 1 μM, the RNase activity decreased and as the concentration of RNP was reduced from 4 nm, RNase activity also declined.

The N-terminal maltose binding protein (MBP) fusions of each of these variants were also prepared and tested for their non-specific RNase activity; however, activity substantially decreased and required more than 3 hours and a 15-fold increase in LbuCas13a RNP concentration to detect nucleic acid degradation by this assay (Table 5). These proteins were purified with a CTD-6× histidine tag or NTD-MBP.

TABLE 5
MBP-Cas13a RNP activity assay data after 3 hr at 37° C.

| Lbu | | Lsh | | Lwa | |
|---|---|---|---|---|---|
| RNP (nM) | Emission | RNP (nM) | Emission | RNP (nM) | Emission |
| 1000 | 550 | 1000 | 415 | 1000 | 362 |
| 500 | 135 | 500 | 319 | 500 | 2589 |
| 250 | 446 | 250 | 239 | 250 | 2173 |
| 125 | 777 | 125 | 196 | 125 | 2058 |
| 63 | 841 | 63 | 174 | 63 | 1874 |
| 31 | 179 | 31 | 168 | 31 | 1747 |
| 16 | 681 | 16 | 166 | 16 | 163 |
| 8 | 354 | 8 | 160 | 8 | 164 |
| 4 | 254 | 4 | 160 | 4 | 166 |
| 2 | 203 | 2 | 161 | 2 | 166 |
| 1 | 177 | 1 | 160 | 1 | 159 |
| 0 | 541 | 0 | 400 | 0 | 368 |

Using the Basic Local Alignment Search Tool (BLAST) on NCBI, the LwaCas13a protein sequence found in the literature [2] had a mutation at position 403; therefore, LwaCas13a G403D was cloned, overexpressed and purified (SEQ ID NO: 13-14). These results (Table 3) reveal RNase activity for this variant using an RNP concentration range between 62.5-500 nM. Although this single mutation uncovered the non-specific RNase activity of this enzyme, LbuCas13a is still the better alternative in terms of the desired activity per molecule of protein.

### Example 3

The ribonucleoprotein (RNP) complex was formed by combining purified Cas13a protein and the corresponding crRNA and incubating at room temperature for 10 minutes.

The Cas13a RNP complex (1 μM) was added to 25 ng of total human RNA (purified from HEK 293), 1 μL RNase Inhibitor, 20 ng of nucleic acid target, 0.2 μM of RNA degradation reporter probe (FAM-IBFQ labeled) in a final volume of 100 μL in nuclease assay buffer (40 mM Tris·HCl, 60 mM NaCl, 6 mM MgCl₂, ph 7.4). The mixture was incubated at 37° C. for 10 min. Following incubation, the reaction mixture was visualized by a fluorescent plate reader (490 nm excitation, 520 nm emission).

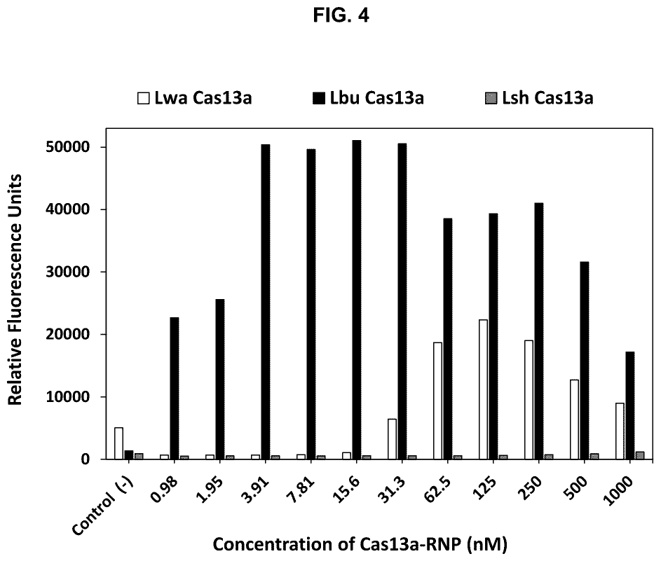

FIG. 4 shows the activity of Lwa Cas13a, Lbu Cas13a, and Lsh Cas13a variants. Lbu Cas13a is active across a broad range of concentrations with peak activity from about 3.91 nM to 31.3 nM. Lwa Cas13a shows activity across a range of concentrations with peak activity from about 62.5 nM to 250 nM.

What is claimed:

1. A method for expressing and purifying a Cas13a protein, the method comprising:
(a) inserting a nucleotide sequence encoding a polypeptide having the polypeptide sequence of SEQ ID NO: 2 or 4 into an expression plasmid;
(b) transforming one or more cells with the expression plasmid;
(c) inducing expression of the transformed plasmid;
(d) isolating the cells;
(e) extracting the Cas13a protein; and
(f) purifying the protein using affinity purification and ion exchange purification.

2. The method of claim 1, wherein the cell comprises E. coli BL21(DE3).

3. The method of claim 1, wherein the expression plasmid comprises pET28 or pET28-MBP-TEV plasmids.

4. The method of claim 1, wherein the nucleotide sequence is inserted into the expression plasmid using isothermal assembly.

6. The method of claim 1, wherein the affinity purification comprises a nickel or a maltose affinity media.

7. The method of claim 1, wherein the affinity purification comprises affinity chromatography comprising:
(f)(1) equilibrating a nickel affinity column with a binding buffer and loading the extracted Cas13a protein;
(f)(2) washing the nickel affinity column with a wash buffer; and
(f)(3) eluting the affinity purified Cas13a protein from the nickel affinity column using an elution buffer.

8. The method of claim 1, wherein the affinity purification comprises affinity chromatography comprising:
(f)(1) equilibrating a maltose affinity column with a binding buffer and loading the extracted Cas13a protein;
(f)(2) washing the maltose affinity column with a wash buffer; and
(f)(3) eluting the affinity purified Cas13a protein from the maltose affinity column using an elution buffer.

9. The method of claim 1, wherein the ion exchange purification comprises a cation exchange media.

10. The method of claim 1, wherein the ion exchange purification comprises cation exchange chromatography comprising:
(f)(1) equilibrating a cation exchange column with a binding buffer and loading the extracted Cas13a protein;
(f)(2) washing the cation exchange column with a wash buffer; and
(f)(3) eluting the cation exchange purified Cas13a protein from the cation exchange column using an elution buffer.

11. The method of claim 1, further comprising concentrating the purified Cas13a protein to approximately 10 mg/mL.

12. The method of claim 11, further comprising dialyzing the concentrated purified Cas13a protein.

13. A method for purifying a recombinant Cas13a protein, the method comprising:
(a) providing an expressed recombinant Cas13a protein having the polypeptide sequence of SEQ ID NO: 2 or 4;
(b) performing an affinity purification comprising a nickel affinity media or a maltose affinity media;
(c) performing an ion exchange purification comprising a cation exchange media; and
(d) collecting the purified Cas13a protein.

14. The method of claim 13, further comprising concentrating the purified Cas13a protein to approximately 10 mg/mL.

15. The method of claim 14, further comprising dialyzing the concentrated purified Cas13a protein against three rounds of dialysis buffer.


SEQUENCE LISTING (44 sequences)

SEQ ID NO: 1 - DNA, 3546 bp, synthetic construct
  misc_feature 1..3546: note = Synthetic
  source 1..3546: mol_type = other DNA
atgaaggtgaccaaagttggtggtatcagccataaaaagtataccagcgaaggtcgtctggttaaaagcgaaagcgaagaaaatcgtaccgatgaacgtctgagcgcactgctgaatatgcgtctggatatgtatatcaaaaatccgagcagcaccgaaaccaaagaaaatcagaaacgtatcggcaagctgaaaaagttcttcagcaacaaaatggtgtacctgaaagataacaccctgagcctgaaaaacggcaagaaagaaaatatcgatcgcgagtatagcgaaaccgatattctggaaagtgatgtgcgtgacaaaaaaaactttgccgtcctgaaaaagatctatctgaacgaaaatgtgaacagcgaagaactggaagtgtttcgcaacgacattaaaaagaagctgaacaagatcaacagcctgaaatatagcttcgagaaaaacaaagccaactatcagaagatcaacgagaacaacatcgaaaaagtggaaggtaaaagcaagcgcaacatcatctatgattattatcgtgaaagcgccaaacgtgatgcctatgttagcaatgttaaagaggccttcgacaagctgtataaagaagaagatattgccaaactggtgctggaaattgaaaatctgaccaagctggaaaaatacaagatccgcgaattctatcacgaaatcattggtcgcaaaaacgataaagagaacttcgccaaaatcatctacgaagaaattcagaacgtgaataacatgaaagaactgatcgagaaagttccggatatgagcgaactgaaaaaaagccaggtgttctacaaatattacctggacaaagaggaactgaacgataaaaacatcaaatacgccttttgccacttcgtggaaatcgaaatgagccagctgctgaaaaactatgtgtataaacgcctgagcaacatcagcaacgataagattaaacgcatcttcgagtaccagaacctgaagaaactgattgaaaacaaactgcttaacaaactggatacctatgtgcgtaattgcggcaaatacaactattatctgcaggatggtgaaattgcgaccagcgattttattgcacgtaatcgtcagaatgaagcctttctgcgtaacattattggtgttagcagcgttgcatattttagcctgcgtaatatcctggaaaccgaaaacgagaatgatatcaccggtcgtatgcgtggtaaaaccgtgaaaaacaataaaggcgaagagaaatatgtgagcggtgaggtggataaaatctacaacgaaaacaaaaagaacgaagtgaaagaaaacctgaaaatgttttacagctacgactttaacatggacaacaagaacgagatcgaagattttttcgccaacattgatgaagccattagcagcattcgtcatggcattgttcactttaatctggaacttgagggcaaagacatcttcgcgtttaaaaacattgcaccgagcgagattagcaaaaagatgttccagaacgaaattaacgagaaaaaactgaaactgaagatctttcgccagctgaatagcgcaaatgtttttcgctatcttgagaaatacaaaatcctgaactatctgaaacgcacccgctttgaatttgtgaacaaaaacattccgtttgtgccgagctttaccaaactgtatagccgtattgatgatctgaaaaacagcctgggcatttattggaaaaccccgaaaaccaacgatgataacaagacgaaagaaatcatcgatgcccagatttatctgcttaagaacatctactatggcgaatttctgaactattttatgagcaacaacggcaacttctttgaaatcagcaaagagattatcgagctgaataaaaacgacaaacgcaatctgaaaaccggcttctataaactgcagaagtttgaggatatccaagaaaagatcccgaaagaatatctggcgaatattcagagcctgtacatgattaatgcaggcaatcaggatgaggaagagaaagatacctatatcgatttcatccagaaaatctttctgaaaggctttatgacctatctggccaataatggtcgtctgagtctgatttatatcggtagtgatgaagaaaccaataccagcctggcagaaaaaaaacaagagttcgataagttcctgaagaagtacgaacagaacaacaacatcaagatcccgtatgaaatcaatgaatttctgcgcgaaatcaagctgggcaacattctgaaatacaccgaacgcctgaatatgttctatctgattctgaaactgctgaaccataaagagctgacgaatctgaaaggtagcctggaaaagtatcagagcgcaaataaagaggaagcatttagcgatcagctggaactgattaatctgctgaatctggataataaccgtgtgaccgaagatttcgaattagaagcagatgagatcggcaaattcctggattttaatggcaacaaagtgaaggacaacaaagagcttaagaagttcgacaccaacaagatctattttgatggcgagaacatcatcaaacaccgtgccttttataacatcaaaaaatacggtatgctgaacctgctggaaaagattgcagataaagcaggctataaaatcagcattgaagagttgaaaaaatacagcaacaagaaaaacgagattgagaaaaaccacaaaatgcaagaaaatctgcaccgcaaatatgcacgtccgcgtaaagatgaaaaattcaccgatgaagattatgaaagctacaaacaggccatcgaaaacatcgaagaatatacccatctgaagaacaaagtcgaattcaacgaactgaatctgctgcagggtctgctgctgcgtattctgcatcgtctggtgggttataccagcatttgggaacgtgatctgcgttttcgcctgaaaggtgaatttcctgaaaaccagtatatcgaggaaatcttcaacttcgagaataaaaagaatgtgaagtataaaggtggccagatcgtcgagaaatatatcaaattctacaaagaactgcaccagaacgacgaggtgaaaatcaacaaatatagcagcgcgaacatcaaagtgctgaaacaagagaaaaaagacctgtacatccgcaactatatcgcccactttaactatattccgcatgcagaaattagtctgctggaagttctggaaaacctgcgtaaactgctgtcatatgatcgtaaacttaaaaacgccgtgatgaaaagcgttgtggacatcctgaaagagtatggttttgttgcgacctttaaaatcggtgccgataaaaagattggtattcagaccctggaaagcgagaagattgttcacctgaaaaatcttaagaaaaagaaacttatgaccgatcgcaatagcgaggaactgtgtaaactggtgaaaattatgtttgagtataaaatggaagagaagaaatccgaaaatggggatccgaattcgagctccgtcgacaagcttgcggccgcactcgagcaccaccaccaccaccactga

SEQ ID NO: 2 - protein, 1181 aa, synthetic construct
  REGION 1..1181: note = Synthetic
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSENGDPNSSSVDKLAAALEHHHHHH

SEQ ID NO: 3 - DNA, 4701 bp, synthetic construct
  misc_feature 1..4701: note = Synthetic
  source 1..4701: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggaaggtgaccaaagttggtggtatcagccataaaaagtataccagcgaaggtcgtctggttaaaagcgaaagcgaagaaaatcgtaccgatgaacgtctgagcgcactgctgaatatgcgtctggatatgtatatcaaaaatccgagcagcaccgaaaccaaagaaaatcagaaacgtatcggcaagctgaaaaagttcttcagcaacaaaatggtgtacctgaaagataacaccctgagcctgaaaaacggcaagaaagaaaatatcgatcgcgagtatagcgaaaccgatattctggaaagtgatgtgcgtgacaaaaaaaactttgccgtcctgaaaaagatctatctgaacgaaaatgtgaacagcgaagaactggaagtgtttcgcaacgacattaaaaagaagctgaacaagatcaacagcctgaaatatagcttcgagaaaaacaaagccaactatcagaagatcaacgagaacaacatcgaaaaagtggaaggtaaaagcaagcgcaacatcatctatgattattatcgtgaaagcgccaaacgtgatgcctatgttagcaatgttaaagaggccttcgacaagctgtataaagaagaagatattgccaaactggtgctggaaattgaaaatctgaccaagctggaaaaatacaagatccgcgaattctatcacgaaatcattggtcgcaaaaacgataaagagaacttcgccaaaatcatctacgaagaaattcagaacgtgaataacatgaaagaactgatcgagaaagttccggatatgagcgaactgaaaaaaagccaggtgttctacaaatattacctggacaaagaggaactgaacgataaaaacatcaaatacgccttttgccacttcgtggaaatcgaaatgagccagctgctgaaaaactatgtgtataaacgcctgagcaacatcagcaacgataagattaaacgcatcttcgagtaccagaacctgaagaaactgattgaaaacaaactgcttaacaaactggatacctatgtgcgtaattgcggcaaatacaactattatctgcaggatggtgaaattgcgaccagcgattttattgcacgtaatcgtcagaatgaagcctttctgcgtaacattattggtgttagcagcgttgcatattttagcctgcgtaatatcctggaaaccgaaaacgagaatgatatcaccggtcgtatgcgtggtaaaaccgtgaaaaacaataaaggcgaagagaaatatgtgagcggtgaggtggataaaatctacaacgaaaacaaaaagaacgaagtgaaagaaaacctgaaaatgttttacagctacgactttaacatggacaacaagaacgagatcgaagattttttcgccaacattgatgaagccattagcagcattcgtcatggcattgttcactttaatctggaacttgagggcaaagacatcttcgcgtttaaaaacattgcaccgagcgagattagcaaaaagatgttccagaacgaaattaacgagaaaaaactgaaactgaagatctttcgccagctgaatagcgcaaatgtttttcgctatcttgagaaatacaaaatcctgaactatctgaaacgcacccgctttgaatttgtgaacaaaaacattccgtttgtgccgagctttaccaaactgtatagccgtattgatgatctgaaaaacagcctgggcatttattggaaaaccccgaaaaccaacgatgataacaagacgaaagaaatcatcgatgcccagatttatctgcttaagaacatctactatggcgaatttctgaactattttatgagcaacaacggcaacttctttgaaatcagcaaagagattatcgagctgaataaaaacgacaaacgcaatctgaaaaccggcttctataaactgcagaagtttgaggatatccaagaaaagatcccgaaagaatatctggcgaatattcagagcctgtacatgattaatgcaggcaatcaggatgaggaagagaaagatacctatatcgatttcatccagaaaatctttctgaaaggctttatgacctatctggccaataatggtcgtctgagtctgatttatatcggtagtgatgaagaaaccaataccagcctggcagaaaaaaaacaagagttcgataagttcctgaagaagtacgaacagaacaacaacatcaagatcccgtatgaaatcaatgaatttctgcgcgaaatcaagctgggcaacattctgaaatacaccgaacgcctgaatatgttctatctgattctgaaactgctgaaccataaagagctgacgaatctgaaaggtagcctggaaaagtatcagagcgcaaataaagaggaagcatttagcgatcagctggaactgattaatctgctgaatctggataataaccgtgtgaccgaagatttcgaattagaagcagatgagatcggcaaattcctggattttaatggcaacaaagtgaaggacaacaaagagcttaagaagttcgacaccaacaagatctattttgatggcgagaacatcatcaaacaccgtgccttttataacatcaaaaaatacggtatgctgaacctgctggaaaagattgcagataaagcaggctataaaatcagcattgaagagttgaaaaaatacagcaacaagaaaaacgagattgagaaaaaccacaaaatgcaagaaaatctgcaccgcaaatatgcacgtccgcgtaaagatgaaaaattcaccgatgaagattatgaaagctacaaacaggccatcgaaaacatcgaagaatatacccatctgaagaacaaagtcgaattcaacgaactgaatctgctgcagggtctgctgctgcgtattctgcatcgtctggtgggttataccagcatttgggaacgtgatctgcgttttcgcctgaaaggtgaatttcctgaaaaccagtatatcgaggaaatcttcaacttcgagaataaaaagaatgtgaagtataaaggtggccagatcgtcgagaaatatatcaaattctacaaagaactgcaccagaacgacgaggtgaaaatcaacaaatatagcagcgcgaacatcaaagtgctgaaacaagagaaaaaagacctgtacatccgcaactatatcgcccactttaactatattccgcatgcagaaattagtctgctggaagttctggaaaacctgcgtaaactgctgtcatatgatcgtaaacttaaaaacgccgtgatgaaaagcgttgtggacatcctgaaagagtatggttttgttgcgacctttaaaatcggtgccgataaaaagattggtattcagaccctggaaagcgagaagattgttcacctgaaaaatcttaagaaaaagaaacttatgaccgatcgcaatagcgaggaactgtgtaaactggtgaaaattatgtttgagtataaaatggaagagaagaaatccgaaaatgatccgaattcgagctccgtcgacaagcttgcggccgcactcgagcaccaccaccaccaccactga

SEQ ID NO: 4 - protein, 1566 aa, synthetic construct
  REGION 1..1566: note = Synthetic
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGRKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSENDPNSSSVDKLAAALEHHHHHH

SEQ ID NO: 5 - DNA, 4236 bp, synthetic construct
  misc_feature 1..4236: note = Synthetic
  source 1..4236: mol_type = other DNA
atgggtaacctgtttggtcataaacgttggtatgaagtgcgcgacaaaaaagactttaaaatcaaacgcaaggtgaaagtgaaacgcaactatgatggcaacaaatatatcctgaacatcaacgagaacaacaacaaagagaagatcgataataataaattcatccgcaaatacatcaactacaaaaaaaacgataacatcctgaaagaattcacccgcaagtttcatgcaggcaacattctgtttaaactgaaaggcaaagaaggcatcattcgcatcgaaaacaatgatgattttctggaaaccgaagaggtggtgctgtatattgaagcatatggcaaaagcgaaaaactgaaggcactgggcattaccaaaaaaaagattatcgatgaagccattcgccagggtattaccaaagatgacaaaaagatcgagatcaagcgccaagaaaacgaagaagaaatcgaaattgatatccgcgacgagtataccaataaaaccctgaatgattgcagcattattctgcgcattatcgagaatgatgagctggaaacgaaaaagagcatctacgagatcttcaaaaacatcaacatgagcctgtacaaaatcatcgagaaaattatcgaaaacgaaaccgagaaggtgttcgagaatcgctattatgaagaacatctgcgtgagaaactgctgaaagatgataaaattgatgtgatcctgaccaacttcatggaaatccgcgaaaagattaaaagcaacctggaaattctgggcttcgtgaaattctatctgaatgttggtggcgacaagaaaaaaagcaagaacaagaaaatgctggtcgaaaaaattctgaacattaacgttgatctgaccgtggaagatattgccgattttgtgattaaagagctggaattctggaacatcaccaaacgcattgagaaggtgaaaaaagtgaacaacgagttcctggaaaaacgtcgtaatcgcacctatatcaaaagctatgttctgctggataagcacgagaaattcaaaattgaacgcgagaacaaaaaggacaaaatcgtgaagtttttcgtggaaaatatcaaaaacaacagcatcaaagaaaaaatcgagaagatcctggccgagttcaaaatcgatgaactgatcaaaaagctggaaaaagaactgaaaaaaggcaactgcgataccgaaattttcggcatctttaagaaacactataaagtgaacttcgatagcaaaaaattcagcaaaaagagcgacgaagagaaagagctgtataagatcatttaccgctatctgaaaggccgtattgaaaaaatcctggtgaatgaacagaaagtgcgcctgaaaaaaatggaaaaaattgagattgagaagattctgaacgagagcatcctgagtgagaaaatcctgaaacgtgttaaacagtataccctggaacacattatgtatctgggtaaactgcgccataacgatattgatatgaccaccgttaataccgatgatttcagccgtctgcatgcaaaagaagaactggatctggaactgattaccttttttgcaagcaccaatatggaactgaacaagatctttagccgtgaaaacattaacaacgacgagaacattgatttctttggtggtgatcgcgagaaaaactatgtcctggataaaaagatcctgaatagcaaaatcaagatcatccgcgatctggatttcatcgacaataagaacaacattaccaacaactttattcgcaaatttaccaaaattggcaccaatgaacgcaaccgtattctgcatgccattagcaaagaacgtgatctgcagggcacccaggatgattataacaaagtgattaacatcatccagaacctgaaaatctccgatgaagaagttagcaaagcactgaatctggatgtggtgttcaaagataagaaaaatatcatcaccaagatcaacgatatcaaaatcagcgaagagaacaataacgacatcaaatatctgccgagctttagcaaagttctgccggaaattcttaatctgtatcgcaataacccgaaaaacgaaccgtttgataccatcgaaacagagaaaattgttctgaacgccctgatctatgtgaacaaagaactgtacaagaaactgatcctggaagatgatctggaagagaacgaatcgaaaaacatctttctgcaagagctgaaaaagaccctgggtaacattgatgagatcgatgaaaacatcatcgaaaattactacaagaacgcacagattagcgcaagcaaaggtaataacaaagccatcaaaaaataccagaaaaaggtgatcgaatgctacattggttatctgcgcaaaaactacgaagaactgttcgatttcagcgatttcaaaatgaacatccaagagatcaagaagcagatcaaggacattaacgacaacaaaacctatgaacgcatcaccgttaaaaccagcgataaaaccattgtgatcaacgacgatttcgagtacatcattagcatttttgcactgctgaattccaacgccgtgatcaacaaaattcgcaatcgcttttttgccaccagtgtttggctgaataccagcgaatatcagaacattatcgatatcctggatgagatcatgcagctgaatacactgcgtaatgaatgcattaccgaaaactggaatctgaaccttgaagaatttattcagaaaatgaaagagatcgagaaagacttcgacgacttcaaaatccagaccaaaaaagaaatcttcaacaactactacgaggacatcaaaaataacattctgaccgaattcaaagacgatattaacggctgtgacgtgctggaaaagaagttggaaaagatcgttatcttcgatgacgaaaccaaattcgaaatcgacaaaaagtccaacatccttcaggatgaacagcgtaaactgagcaatatcaacaagaaagacctgaagaagaaggtcgaccagtacatcaaagacaaagaccaagaaattaagagcaaaatcctgtgccgcatcatctttaacagcgactttctgaaaaagtataagaaagagattgacaacctgatcgaggatatggaaagcgagaacgaaaacaagtttcaagagatctactatccgaaagaacgcaaaaacgagctgtacatctacaagaagaacctgttcctgaatattggcaacccgaacttcgacaaaatctatggtctgatcagcaacgacattaaaatggccgatgcaaaattcctgtttaatatcgatggtaaaaacatccgtaaaaacaaaattagcgagatcgacgcgatcctgaaaaacctgaacgataaactgaatggctacagcaaagaatataaagagaaatacattaaaaagctgaaagaaaatgacgacttcttcgccaagaacatccagaataaaaactataaaagcttcgagaaggactacaatcgcgtgtccgaatataagaaaattcgtgatctggtggaattcaactatctgaacaaaatcgaaagctatctgatcgatatcaactggaaactggcaattcagatggcacgttttgagcgtgatatgcactatattgttaatggtctgcgtgaactgggcatcattaaactgagtggttataataccggcattagccgtgcatatccgaaacgtaatggttccgatggtttttataccaccaccgcctattacaaatttttcgacgaagaaagctacaagaaatttgagaaaatttgctacggcttcggcattgatctgagcgaaaatagcgaaattaacaagccggaaaatgagagcattcgcaactatatctcccacttttatatcgtgcgtaatccgtttgccgattatagcattgcagagcagattgatcgtgttagcaatctgctgagctatagtacccgttataacaatagcacctatgccagcgtgtttgaggtgtttaaaaaggatgttaacctggactatgacgagctgaagaaaaagttcaaactgatcggcaacaatgacatcctggaacgtctgatgaaaccgaaaaaagttagtgtgctggaacttgagagctacaacagcgattatatcaagaacctgattatcgagctgctgaccaagattgaaaataccaatgataccctgggggatccgaattcgagctccgtcgacaagcttgcggccgcactcgagcaccaccaccaccaccactga

SEQ ID NO: 6 - protein, 1411 aa, synthetic construct
  REGION 1..1411: note = Synthetic
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTLGDPNSSSVDKLAAALEHHHHHH

SEQ ID NO: 7 - DNA, 5391 bp, synthetic construct
  misc_feature 1..5391: note = Synthetic
  source 1..5391: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggggtaacctgtttggtcataaacgttggtatgaagtgcgcgacaaaaaagactttaaaatcaaacgcaaggtgaaagtgaaacgcaactatgatggcaacaaatatatcctgaacatcaacgagaacaacaacaaagagaagatcgataataataaattcatccgcaaatacatcaactacaaaaaaaacgataacatcctgaaagaattcacccgcaagtttcatgcaggcaacattctgtttaaactgaaaggcaaagaaggcatcattcgcatcgaaaacaatgatgattttctggaaaccgaagaggtggtgctgtatattgaagcatatggcaaaagcgaaaaactgaaggcactgggcattaccaaaaaaaagattatcgatgaagccattcgccagggtattaccaaagatgacaaaaagatcgagatcaagcgccaagaaaacgaagaagaaatcgaaattgatatccgcgacgagtataccaataaaaccctgaatgattgcagcattattctgcgcattatcgagaatgatgagctggaaacgaaaaagagcatctacgagatcttcaaaaacatcaacatgagcctgtacaaaatcatcgagaaaattatcgaaaacgaaaccgagaaggtgttcgagaatcgctattatgaagaacatctgcgtgagaaactgctgaaagatgataaaattgatgtgatcctgaccaacttcatggaaatccgcgaaaagattaaaagcaacctggaaattctgggcttcgtgaaattctatctgaatgttggtggcgacaagaaaaaaagcaagaacaagaaaatgctggtcgaaaaaattctgaacattaacgttgatctgaccgtggaagatattgccgattttgtgattaaagagctggaattctggaacatcaccaaacgcattgagaaggtgaaaaaagtgaacaacgagttcctggaaaaacgtcgtaatcgcacctatatcaaaagctatgttctgctggataagcacgagaaattcaaaattgaacgcgagaacaaaaaggacaaaatcgtgaagtttttcgtggaaaatatcaaaaacaacagcatcaaagaaaaaatcgagaagatcctggccgagttcaaaatcgatgaactgatcaaaaagctggaaaaagaactgaaaaaaggcaactgcgataccgaaattttcggcatctttaagaaacactataaagtgaacttcgatagcaaaaaattcagcaaaaagagcgacgaagagaaagagctgtataagatcatttaccgctatctgaaaggccgtattgaaaaaatcctggtgaatgaacagaaagtgcgcctgaaaaaaatggaaaaaattgagattgagaagattctgaacgagagcatcctgagtgagaaaatcctgaaacgtgttaaacagtataccctggaacacattatgtatctgggtaaactgcgccataacgatattgatatgaccaccgttaataccgatgatttcagccgtctgcatgcaaaagaagaactggatctggaactgattaccttttttgcaagcaccaatatggaactgaacaagatctttagccgtgaaaacattaacaacgacgagaacattgatttctttggtggtgatcgcgagaaaaactatgtcctggataaaaagatcctgaatagcaaaatcaagatcatccgcgatctggatttcatcgacaataagaacaacattaccaacaactttattcgcaaatttaccaaaattggcaccaatgaacgcaaccgtattctgcatgccattagcaaagaacgtgatctgcagggcacccaggatgattataacaaagtgattaacatcatccagaacctgaaaatctccgatgaagaagttagcaaagcactgaatctggatgtggtgttcaaagataagaaaaatatcatcaccaagatcaacgatatcaaaatcagcgaagagaacaataacgacatcaaatatctgccgagctttagcaaagttctgccggaaattcttaatctgtatcgcaataacccgaaaaacgaaccgtttgataccatcgaaacagagaaaattgttctgaacgccctgatctatgtgaacaaagaactgtacaagaaactgatcctggaagatgatctggaagagaacgaatcgaaaaacatctttctgcaagagctgaaaaagaccctgggtaacattgatgagatcgatgaaaacatcatcgaaaattactacaagaacgcacagattagcgcaagcaaaggtaataacaaagccatcaaaaaataccagaaaaaggtgatcgaatgctacattggttatctgcgcaaaaactacgaagaactgttcgatttcagcgatttcaaaatgaacatccaagagatcaagaagcagatcaaggacattaacgacaacaaaacctatgaacgcatcaccgttaaaaccagcgataaaaccattgtgatcaacgacgatttcgagtacatcattagcatttttgcactgctgaattccaacgccgtgatcaacaaaattcgcaatcgcttttttgccaccagtgtttggctgaataccagcgaatatcagaacattatcgatatcctggatgagatcatgcagctgaatacactgcgtaatgaatgcattaccgaaaactggaatctgaaccttgaagaatttattcagaaaatgaaagagatcgagaaagacttcgacgacttcaaaatccagaccaaaaaagaaatcttcaacaactactacgaggacatcaaaaataacattctgaccgaattcaaagacgatattaacggctgtgacgtgctggaaaagaagttggaaaagatcgttatcttcgatgacgaaaccaaattcgaaatcgacaaaaagtccaacatccttcaggatgaacagcgtaaactgagcaatatcaacaagaaagacctgaagaagaaggtcgaccagtacatcaaagacaaagaccaagaaattaagagcaaaatcctgtgccgcatcatctttaacagcgactttctgaaaaagtataagaaagagattgacaacctgatcgaggatatggaaagcgagaacgaaaacaagtttcaagagatctactatccgaaagaacgcaaaaacgagctgtacatctacaagaagaacctgttcctgaatattggcaacccgaacttcgacaaaatctatggtctgatcagcaacgacattaaaatggccgatgcaaaattcctgtttaatatcgatggtaaaaacatccgtaaaaacaaaattagcgagatcgacgcgatcctgaaaaacctgaacgataaactgaatggctacagcaaagaatataaagagaaatacattaaaaagctgaaagaaaatgacgacttcttcgccaagaacatccagaataaaaactataaaagcttcgagaaggactacaatcgcgtgtccgaatataagaaaattcgtgatctggtggaattcaactatctgaacaaaatcgaaagctatctgatcgatatcaactggaaactggcaattcagatggcacgttttgagcgtgatatgcactatattgttaatggtctgcgtgaactgggcatcattaaactgagtggttataataccggcattagccgtgcatatccgaaacgtaatggttccgatggtttttataccaccaccgcctattacaaatttttcgacgaagaaagctacaagaaatttgagaaaatttgctacggcttcggcattgatctgagcgaaaatagcgaaattaacaagccggaaaatgagagcattcgcaactatatctcccacttttatatcgtgcgtaatccgtttgccgattatagcattgcagagcagattgatcgtgttagcaatctgctgagctatagtacccgttataacaatagcacctatgccagcgtgtttgaggtgtttaaaaaggatgttaacctggactatgacgagctgaagaaaaagttcaaactgatcggcaacaatgacatcctggaacgtctgatgaaaccgaaaaaagttagtgtgctggaacttgagagctacaacagcgattatatcaagaacctgattatcgagctgctgaccaagattgaaaataccaatgataccctggatccgaattcgagctccgtcgacaagcttgcggccgcactcgagcaccaccaccaccaccactga

SEQ ID NO: 8 - protein, 1796 aa, synthetic construct
  REGION 1..1796: note = Synthetic
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGRGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTLDPNSSSVDKLAAALEHHHHHH

SEQ ID NO: 9 - DNA, 3525 bp, synthetic construct
  misc_feature 1..3525: note = Synthetic
  source 1..3525: mol_type = other DNA
atgaaagtgaccaaagtggatggcatcagccacaaaaaatacatcgaagaaggcaaactggttaaaagcaccagcgaagaaaatcgtaccagcgaacgtctgagcgaactgctgagcattcgtctggatatctatatcaaaaatccggataatgccagcgaggaagaaaaccgtattcgtcgtgaaaacctgaaaaagttcttcagcaataaagtgctgcacctgaaagatagcgttctgtatctgaaaaaccgcaaagaaaaaaatgccgtgcaggacaaaaactatagcgaagaggatatcagcgagtatgacctgaagaacaaaaatagctttagcgtgctgaaaaaaatcctgctgaatgaagatgtgaatagcgaggaactggaaatctttcgtaaagatgttgaagccaagctgaacaaaatcaacagcctgaaatatagctttgaagaaaacaaggccaactatcagaaaatcaacgagaacaacgtggaaaaagttggtggtaaaagcaaacgcaacatcatctatgattattatcgcgaaagcgcgaaacgcaacgattatatcaataatgtgcaagaggccttcgacaaactgtacaaaaaagaggacatcgaaaaactgttttttctgatcgagaacagcaagaagcacgagaaatacaaaatccgcgagtactaccataaaatcatcggtcgcaaaaacgataaagagaacttcgccaaaatcatctacgaagaaattcagaacgtgaacaacatcaaagaactgatcgaaaaaattccggacatgagcgagctgaagaaaagccaggtgttctataaatactacctggacaaagaggaactgaacgacaaaaacatcaaatatgccttttgccacttcgtcgaaattgaaatgagccagctgcttaaaaactacgtgtataaacgcctgagcaacatcagcaacgataaaatcaaacgtatctttgaatatcagaatctgaagaaactgattgaaaacaaactgctgaacaagctggatacctatgttcgtaattgcggcaaatacaactactatctgcaggttggtgaaattgcaaccagcgattttattgcacgtaatcgtcagaatgaagcctttctgcgtaacattattggtgttagcagcgttgcatattttagcctgcgtaatattctggaaaccgaaaacgaaaatggcattaccggtcgtatgcgtggtaaaaccgttaaaaacaataaaggcgaagagaagtatgtgagcggtgaagtggataaaatctataacgaaaacaagcagaacgaagtgaaagaaaatctgaaaatgttttacagctacgacttcaacatggacaacaaaaacgagatcgaagatttcttcgccaacattgatgaagccattagcagtattcgtcatggcattgtgcactttaatctggaacttgaaggcaaagacatcttcgcgtttaaaaacattgcaccgagcgagatcagcaaaaaaatgtttcagaacgagattaacgaaaaaaaactgaaactgaaaatcttcaaacagctgaatagcgccaacgtgttcaactattatgagaaagacgtgatcatcaaataccttaaaaacaccaaattcaacttcgtgaataaaaacatcccgtttgttccgagcttcaccaaactgtataacaaaattgaagatctgcgcaataccctgaagtttttttggagcgttccgaaagacaaagaagaaaaagacgcacagatctacctgcttaagaacatctattatggcgaatttctgaacaaattcgtgaaaaatagcaaagtgttcttcaaaatcaccaacgaggtgatcaagattaacaaacagcgtaatcagaaaaccggtcactacaaataccagaagtttgagaacattgaaaaaaccgtgccggttgaatatctggcaattattcagagccgtgagatgattaacaaccaggataaagaagagaaaaacacctacatcgatttcatccagcagatctttctgaaaggctttatcgattacctgaacaagaacaacctgaagtatatcgagtcgaacaacaataacgacaacaacgacatctttagcaaaatcaaaatcaagaaagataataaagaaaaatacgacaagatcctgaaaaactatgagaagcacaaccgcaacaaagaaattccgcatgagatcaatgaatttgtgcgcgaaattaaactgggcaaaatcctgaaatacaccgagaacctgaatatgttctatctgattctgaagctgctgaaccataaagagctgaccaatctgaaaggtagcctggaaaaatatcagagcgcaaacaaagaagagacattttctgacgaactggaactgattaatctgctgaatctggataataaccgtgtgaccgaagattttgaactggaagcaaatgaaatcggcaaattcctggatttcaatgagaacaaaattaaggaccggaaagagcttaaaaagtttgataccaacaaaatctacttcgacggcgagaacattatcaaacatcgtgccttttataacatcaaaaagtatggcatgctgaacctgctggaaaaaattgcagataaagccaagtacaaaattagcctgaaagaacttaaagagtacagcaacaaaaagaacgaaatcgagaagaactataccatgcagcagaatctgcatcgtaaatatgcacgtccgaaaaaagacgagaaattcaacgatgaggactataaagaatacgagaaagccattggcaacatccagaaatatacccacttgaaaaacaaagtggaatttaacgagctgaatttactgcagggtctgctgctgaaaattctgcaccgtctggttggttataccagcatttgggaacgtgatctgcgttttcgcctgaaaggtgaatttcctgaaaaccactatatcgaggaaattttcaactttgacaacagcaaaaacgtgaaatataagagcggtcagatcgtcgaaaagtacatcaacttttacaaagaactttacaaggataatgtggaaaaacgcagcatctacagcgacaagaaagtgaaaaagctgaagcaagaaaagaaagacctgtacatccgtaattatatcgcccactttaactatatcccgcatgcagaaattagtctgctggaagttctggaaaatctgcgtaaactgctgtcatatgatcgcaaactgaagaacgcaatcatgaaaagcattgtggatatcctgaaagagtatggttttgtcgccacctttaaaatcggtgccgataagaaaattgagattcagaccctggaaagcgagaaaattgtgcatcttaagaaccttaaaaagaaaaaactgatgaccgatcgcaacagcgaagagttatgtgaactggtgaaagtgatgttcgaatacaaagcactggaaggggatccgaattcgagctccgtcgacaagcttgcggccgcactcgagcaccaccaccaccaccactga

SEQ ID NO: 10 - protein, 1174 aa, synthetic construct
  REGION 1..1174: note = Synthetic
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENGITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALEGDPNSSSVDKLAAALEHHHHHH

SEQ ID NO: 11 - DNA, 4680 bp, synthetic construct
  misc_feature 1..4680: note = Synthetic
  source 1..4680: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggaaagtgaccaaagtggatggcatcagccacaaaaaatacatcgaagaaggcaaactggttaaaagcaccagcgaagaaaatcgtaccagcgaacgtctgagcgaactgctgagcattcgtctggatatctatatcaaaaatccggataatgccagcgaggaagaaaaccgtattcgtcgtgaaaacctgaaaaagttcttcagcaataaagtgctgcacctgaaagatagcgttctgtatctgaaaaaccgcaaagaaaaaaatgccgtgcaggacaaaaactatagcgaagaggatatcagcgagtatgacctgaagaacaaaaatagctttagcgtgctgaaaaaaatcctgctgaatgaagatgtgaatagcgaggaactggaaatctttcgtaaagatgttgaagccaagctgaacaaaatcaacagcctgaaatatagctttgaagaaaacaaggccaactatcagaaaatcaacgagaacaacgtggaaaaagttggtggtaaaagcaaacgcaacatcatctatgattattatcgcgaaagcgcgaaacgcaacgattatatcaataatgtgcaagaggccttcgacaaactgtacaaaaaagaggacatcgaaaaactgttttttctgatcgagaacagcaagaagcacgagaaatacaaaatccgcgagtactaccataaaatcatcggtcgcaaaaacgataaagagaacttcgccaaaatcatctacgaagaaattcagaacgtgaacaacatcaaagaactgatcgaaaaaattccggacatgagcgagctgaagaaaagccaggtgttctataaatactacctggacaaagaggaactgaacgacaaaaacatcaaatatgccttttgccacttcgtcgaaattgaaatgagccagctgcttaaaaactacgtgtataaacgcctgagcaacatcagcaacgataaaatcaaacgtatctttgaatatcagaatctgaagaaactgattgaaaacaaactgctgaacaagctggatacctatgttcgtaattgcggcaaatacaactactatctgcaggttggtgaaattgcaaccagcgattttattgcacgtaatcgtcagaatgaagcctttctgcgtaacattattggtgttagcagcgttgcatattttagcctgcgtaatattctggaaaccgaaaacgaaaatggtattaccggtcgtatgcgtggtaaaaccgttaaaaacaataaaggcgaagagaagtatgtgagcggtgaagtggataaaatctataacgaaaacaagcagaacgaagtgaaagaaaatctgaaaatgttttacagctacgacttcaacatggacaacaaaaacgagatcgaagatttcttcgccaacattgatgaagccattagcagtattcgtcatggcattgtgcactttaatctggaacttgaaggcaaagacatcttcgcgtttaaaaacattgcaccgagcgagatcagcaaaaaaatgtttcagaacgagattaacgaaaaaaaactgaaactgaaaatcttcaaacagctgaatagcgccaacgtgttcaactattatgagaaagacgtgatcatcaaataccttaaaaacaccaaattcaacttcgtgaataaaaacatcccgtttgttccgagcttcaccaaactgtataacaaaattgaagatctgcgcaataccctgaagtttttttggagcgttccgaaagacaaagaagaaaaagacgcacagatctacctgcttaagaacatctattatggcgaatttctgaacaaattcgtgaaaaatagcaaagtgttcttcaaaatcaccaacgaggtgatcaagattaacaaacagcgtaatcagaaaaccggtcactacaaataccagaagtttgagaacattgaaaaaaccgtgccggttgaatatctggcaattattcagagccgtgagatgattaacaaccaggataaagaagagaaaaacacctacatcgatttcatccagcagatctttctgaaaggctttatcgattacctgaacaagaacaacctgaagtatatcgagtcgaacaacaataacgacaacaacgacatctttagcaaaatcaaaatcaagaaagataataaagaaaaatacgacaagatcctgaaaaactatgagaagcacaaccgcaacaaagaaattccgcatgagatcaatgaatttgtgcgcgaaattaaactgggcaaaatcctgaaatacaccgagaacctgaatatgttctatctgattctgaagctgctgaaccataaagagctgaccaatctgaaaggtagcctggaaaaatatcagagcgcaaacaaagaagagacattttctgacgaactggaactgattaatctgctgaatctggataataaccgtgtgaccgaagattttgaactggaagcaaatgaaatcggcaaattcctggatttcaatgagaacaaaattaaggaccggaaagagcttaaaaagtttgataccaacaaaatctacttcgacggcgagaacattatcaaacatcgtgccttttataacatcaaaaagtatggcatgctgaacctgctggaaaaaattgcagataaagccaagtacaaaattagcctgaaagaacttaaagagtacagcaacaaaaagaacgaaatcgagaagaactataccatgcagcagaatctgcatcgtaaatatgcacgtccgaaaaaagacgagaaattcaacgatgaggactataaagaatacgagaaagccattggcaacatccagaaatatacccacttgaaaaacaaagtggaatttaacgagctgaatttactgcagggtctgctgctgaaaattctgcaccgtctggttggttataccagcatttgggaacgtgatctgcgttttcgcctgaaaggtgaatttcctgaaaaccactatatcgaggaaattttcaactttgacaacagcaaaaacgtgaaatataagagcggtcagatcgtcgaaaagtacatcaacttttacaaagaactttacaaggataatgtggaaaaacgcagcatctacagcgacaagaaagtgaaaaagctgaagcaagaaaagaaagacctgtacatccgtaattatatcgcccactttaactatatcccgcatgcagaaattagtctgctggaagttctggaaaatctgcgtaaactgctgtcatatgatcgcaaactgaagaacgcaatcatgaaaagcattgtggatatcctgaaagagtatggttttgtcgccacctttaaaatcggtgccgataagaaaattgagattcagaccctggaaagcgagaaaattgtgcatcttaagaaccttaaaaagaaaaaactgatgaccgatcgcaacagcgaagagttatgtgaactggtgaaagtgatgttcgaatacaaagcactggaagatccgaattcgagctccgtcgacaagcttgcggccgcactcgagcaccaccaccaccaccactga

SEQ ID NO: 12 - protein, 1559 aa, synthetic construct
  REGION 1..1559: note = Synthetic
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGRKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENGITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALEDPNSSSVDKLAAALEHHHHHH

SEQ ID NO: 13 - DNA, 3525 bp, synthetic construct
  misc_feature 1..3525: note = Synthetic
  source 1..3525: mol_type = other DNA
atgaaagtgaccaaagtggatggcatcagccacaaaaaatacatcgaagaaggcaaactggttaaaagcaccagcgaagaaaatcgtaccagcgaacgtctgagcgaactgctgagcattcgtctggatatctatatcaaaaatccggataatgccagcgaggaagaaaaccgtattcgtcgtgaaaacctgaaaaagttcttcagcaataaagtgctgcacctgaaagatagcgttctgtatctgaaaaaccgcaaagaaaaaaatgccgtgcaggacaaaaactatagcgaagaggatatcagcgagtatgacctgaagaacaaaaatagctttagcgtgctgaaaaaaatcctgctgaatgaagatgtgaatagcgaggaactggaaatctttcgtaaagatgttgaagccaagctgaacaaaatcaacagcctgaaatatagctttgaagaaaacaaggccaactatcagaaaatcaacgagaacaacgtggaaaaagttggtggtaaaagcaaacgcaacatcatctatgattattatcgcgaaagcgcgaaacgcaacgattatatcaataatgtgcaagaggccttcgacaaactgtacaaaaaagaggacatcgaaaaactgttttttctgatcgagaacagcaagaagcacgagaaatacaaaatccgcgagtactaccataaaatcatcggtcgcaaaaacgataaagagaacttcgccaaaatcatctacgaagaaattcagaacgtgaacaacatcaaagaactgatcgaaaaaattccggacatgagcgagctgaagaaaagccaggtgttctataaatactacctggacaaagaggaactgaacgacaaaaacatcaaatatgccttttgccacttcgtcgaaattgaaatgagccagctgcttaaaaactacgtgtataaacgcctgagcaacatcagcaacgataaaatcaaacgtatctttgaatatcagaatctgaagaaactgattgaaaacaaactgctgaacaagctggatacctatgttcgtaattgcggcaaatacaactactatctgcaggttggtgaaattgcaaccagcgattttattgcacgtaatcgtcagaatgaagcctttctgcgtaacattattggtgttagcagcgttgcatattttagcctgcgtaatattctggaaaccgaaaacgaaaatgatattaccggtcgtatgcgtggtaaaaccgttaaaaacaataaaggcgaagagaagtatgtgagcggtgaagtggataaaatctataacgaaaacaagcagaacgaagtgaaagaaaatctgaaaatgttttacagctacgacttcaacatggacaacaaaaacgagatcgaagatttcttcgccaacattgatgaagccattagcagtattcgtcatggcattgtgcactttaatctggaacttgaaggcaaagacatcttcgcgtttaaaaacattgcaccgagcgagatcagcaaaaaaatgtttcagaacgagattaacgaaaaaaaactgaaactgaaaatcttcaaacagctgaatagcgccaacgtgttcaactattatgagaaagacgtgatcatcaaataccttaaaaacaccaaattcaacttcgtgaataaaaacatcccgtttgttccgagcttcaccaaactgtataacaaaattgaagatctgcgcaataccctgaagtttttttggagcgttccgaaagacaaagaagaaaaagacgcacagatctacctgcttaagaacatctattatggcgaatttctgaacaaattcgtgaaaaatagcaaagtgttcttcaaaatcaccaacgaggtgatcaagattaacaaacagcgtaatcagaaaaccggtcactacaaataccagaagtttgagaacattgaaaaaaccgtgccggttgaatatctggcaattattcagagccgtgagatgattaacaaccaggataaagaagagaaaaacacctacatcgatttcatccagcagatctttctgaaaggctttatcgattacctgaacaagaacaacctgaagtatatcgagtcgaacaacaataacgacaacaacgacatctttagcaaaatcaaaatcaagaaagataataaagaaaaatacgacaagatcctgaaaaactatgagaagcacaaccgcaacaaagaaattccgcatgagatcaatgaatttgtgcgcgaaattaaactgggcaaaatcctgaaatacaccgagaacctgaatatgttctatctgattctgaagctgctgaaccataaagagctgaccaatctgaaaggtagcctggaaaaatatcagagcgcaaacaaagaagagacattttctgacgaactggaactgattaatctgctgaatctggataataaccgtgtgaccgaagattttgaactggaagcaaatgaaatcggcaaattcctggatttcaatgagaacaaaattaaggaccggaaagagcttaaaaagtttgataccaacaaaatctacttcgacggcgagaacattatcaaacatcgtgccttttataacatcaaaaagtatggcatgctgaacctgctggaaaaaattgcagataaagccaagtacaaaattagcctgaaagaacttaaagagtacagcaacaaaaagaacgaaatcgagaagaactataccatgcagcagaatctgcatcgtaaatatgcacgtccgaaaaaagacgagaaattcaacgatgaggactataaagaatacgagaaagccattggcaacatccagaaatatacccacttgaaaaacaaagtggaatttaacgagctgaatttactgcagggtctgctgctgaaaattctgcaccgtctggttggttataccagcatttgggaacgtgatctgcgttttcgcctgaaaggtgaatttcctgaaaaccactatatcgaggaaattttcaactttgacaacagcaaaaacgtgaaatataagagcggtcagatcgtcgaaaagtacatcaacttttacaaagaactttacaaggataatgtggaaaaacgcagcatctacagcgacaagaaagtgaaaaagctgaagcaagaaaagaaagacctgtacatccgtaattatatcgcccactttaactatatcccgcatgcagaaattagtctgctggaagttctggaaaatctgcgtaaactgctgtcatatgatcgcaaactgaagaacgcaatcatgaaaagcattgtggatatcctgaaagagtatggttttgtcgccacctttaaaatcggtgccgataagaaaattgagattcagaccctggaaagcgagaaaattgtgcatcttaagaaccttaaaaagaaaaaactgatgaccgatcgcaacagcgaagagttatgtgaactggtgaaagtgatgttcgaatacaaagcactggaaggggatccgaattcgagctccgtcgacaagcttgcggccgcactcgagcaccaccaccaccaccactga

SEQ ID NO: 14 - protein, 1174 aa, synthetic construct
  REGION 1..1174: note = Synthetic
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALEGDPNSSSVDKLAAALEHHHHHH

SEQ ID NO: 15 - DNA, 60 bp, synthetic construct
  misc_feature 1..60: note = Synthetic
  source 1..60: mol_type = other DNA
gaaataattttgtttaactttaagaaggagatataccatgaaggtgaccaaagttggtgg

SEQ ID NO: 16 - DNA, 73 bp, synthetic construct
  misc_feature 1..73: note = Synthetic
  source 1..73: mol_type = other DNA
cggccgcaagcttgtcgacggagctcgaattcggatccccattttcggatttcttctcttccattttatactc

SEQ ID NO: 17 - DNA, 60 bp, synthetic construct
  misc_feature 1..60: note = Synthetic
  source 1..60: mol_type = other DNA
aataacaataacaacaacctcgggatcgagggaaggaaggtgaccaaagttggtggtatc

SEQ ID NO: 18 - DNA, 73 bp, synthetic construct
  misc_feature 1..73: note = Synthetic
  source 1..73: mol_type = other DNA
gtgcggccgcaagcttgtcgacggagctcgaattcggatcattttcggatttcttctcttccattttatactc

SEQ ID NO: 19 - DNA, 60 bp, synthetic construct
  misc_feature 1..60: note = Synthetic
  source 1..60: mol_type = other DNA
ataattttgtttaactttaagaaggagatataccatgggtaacctgtttggtcataaacg

SEQ ID NO: 20 - DNA, 69 bp, synthetic construct
  misc_feature 1..69: note = Synthetic
  source 1..69: mol_type = other DNA
cggccgcaagcttgtcgacggagctcgaattcggatcccccagggtatcattggtattttcaatcttgg

SEQ ID NO: 21 - DNA, 60 bp, synthetic construct
  misc_feature 1..60: note = Synthetic
  source 1..60: mol_type = other DNA
taacaataacaacaacctcgggatcgagggaaggggtaacctgtttggtcataaacgttg

SEQ ID NO: 22 - DNA, 69 bp, synthetic construct
  misc_feature 1..69: note = Synthetic
  source 1..69: mol_type = other DNA
gtgcggccgcaagcttgtcgacggagctcgaattcggatccagggtatcattggtattttcaatcttgg

SEQ ID NO: 23 - DNA, 59 bp, synthetic construct
  misc_feature 1..59: note = Synthetic
  source 1..59: mol_type = other DNA
aaataattttgtttaactttaagaaggagatataccatgaaagtgaccaaagtggatgg

SEQ ID NO: 24 - DNA, 60 bp, synthetic construct
  misc_feature 1..60: note = Synthetic
  source 1..60: mol_type = other DNA
gcaagcttgtcgacggagctcgaattcggatccccttccagtgctttgtattcgaacatc

SEQ ID NO: 25 - DNA, 60 bp, synthetic construct
  misc_feature 1..60: note = Synthetic
  source 1..60: mol_type = other DNA
acaataacaataacaacaacctcgggatcgagggaaggaaagtgaccaaagtggatggca

SEQ ID NO: 26 - DNA, 60 bp, synthetic construct
  misc_feature 1..60: note = Synthetic
  source 1..60: mol_type = other DNA
caagcttgtcgacggagctcgaattcggatccccttccagtgctttgtattcgaacatca

SEQ ID NO: 27 - DNA, 20 bp, synthetic construct
  misc_feature 1..20: note = Synthetic
  source 1..20: mol_type = other DNA
ggggatccgaattcgagctc

SEQ ID NO: 28 - DNA, 37 bp, synthetic construct
  misc_feature 1..37: note = Synthetic
  source 1..37: mol_type = other DNA
ggtatatctccttcttaaagttaaacaaaattatttc

SEQ ID NO: 29 - DNA, 20 bp, synthetic construct
  misc_feature 1..20: note = Synthetic
  source 1..20: mol_type = other DNA
gatccgaattcgagctccgt

SEQ ID NO: 30 - DNA, 19 bp, synthetic construct
  misc_feature 1..19: note = Synthetic
  source 1..19: mol_type = other DNA
ccttccctcgatcccgagg

SEQ ID NO: 31 - DNA, 53 bp, synthetic construct
  misc_feature 1..53: note = Synthetic
  source 1..53: mol_type = other DNA
gtaatattctggaaaccgaaaacgaaaatgatattaccggtcgtatgcgtggt

SEQ ID NO: 32 - DNA, 53 bp, synthetic construct
  misc_feature 1..53: note = Synthetic
  source 1..53: mol_type = other DNA
accacgcatacgaccggtaatatcattttcgttttcggtttccagaatattac

SEQ ID NO: 33 - RNA, 67 nt, synthetic construct
  misc_feature 1..67: note = Synthetic
  source 1..67: mol_type = other RNA
ggggatttagactaccccaaaaacgaaggggactaaaactagattgctgttctaccaagtaatccat

SEQ ID NO: 34 - RNA, 59 nt, synthetic construct
  misc_feature 1..59: note = Synthetic
  source 1..59: mol_type = other RNA
gaccaccccaaaaatgaaggggactaaaacatagattgctgttctaccaagtaatccat

SEQ ID NO: 35 - RNA, 56 nt, synthetic construct
  misc_feature 1..56: note = Synthetic
  source 1..56: mol_type = other RNA
ccaccccaatatcgaaggggactaaaactagattgctgttctaccaagtaatccat

SEQ ID NO: 36 - DNA, 8752 bp, synthetic construct
  misc_feature 1..8752: note = Synthetic
  source 1..8752: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaggtgaccaaagttggtggtatcagccataaaaagtataccagcgaaggtcgtctggttaaaagcgaaagcgaagaaaatcgtaccgatgaacgtctgagcgcactgctgaatatgcgtctggatatgtatatcaaaaatccgagcagcaccgaaaccaaagaaaatcagaaacgtatcggcaagctgaaaaagttcttcagcaacaaaatggtgtacctgaaagataacaccctgagcctgaaaaacggcaagaaagaaaatatcgatcgcgagtatagcgaaaccgatattctggaaagtgatgtgcgtgacaaaaaaaactttgccgtcctgaaaaagatctatctgaacgaaaatgtgaacagcgaagaactggaagtgtttcgcaacgacattaaaaagaagctgaacaagatcaacagcctgaaatatagcttcgagaaaaacaaagccaactatcagaagatcaacgagaacaacatcgaaaaagtggaaggtaaaagcaagcgcaacatcatctatgattattatcgtgaaagcgccaaacgtgatgcctatgttagcaatgttaaagaggccttcgacaagctgtataaagaagaagatattgccaaactggtgctggaaattgaaaatctgaccaagctggaaaaatacaagatccgcgaattctatcacgaaatcattggtcgcaaaaacgataaagagaacttcgccaaaatcatctacgaagaaattcagaacgtgaataacatgaaagaactgatcgagaaagttccggatatgagcgaactgaaaaaaagccaggtgttctacaaatattacctggacaaagaggaactgaacgataaaaacatcaaatacgccttttgccacttcgtggaaatcgaaatgagccagctgctgaaaaactatgtgtataaacgcctgagcaacatcagcaacgataagattaaacgcatcttcgagtaccagaacctgaagaaactgattgaaaacaaactgcttaacaaactggatacctatgtgcgtaattgcggcaaatacaactattatctgcaggatggtgaaattgcgaccagcgattttattgcacgtaatcgtcagaatgaagcctttctgcgtaacattattggtgttagcagcgttgcatattttagcctgcgtaatatcctggaaaccgaaaacgagaatgatatcaccggtcgtatgcgtggtaaaaccgtgaaaaacaataaaggcgaagagaaatatgtgagcggtgaggtggataaaatctacaacgaaaacaaaaagaacgaagtgaaagaaaacctgaaaatgttttacagctacgactttaacatggacaacaagaacgagatcgaagattttttcgccaacattgatgaagccattagcagcattcgtcatggcattgttcactttaatctggaacttgagggcaaagacatcttcgcgtttaaaaacattgcaccgagcgagattagcaaaaagatgttccagaacgaaattaacgagaaaaaactgaaactgaagatctttcgccagctgaatagcgcaaatgtttttcgctatcttgagaaatacaaaatcctgaactatctgaaacgcacccgctttgaatttgtgaacaaaaacattccgtttgtgccgagctttaccaaactgtatagccgtattgatgatctgaaaaacagcctgggcatttattggaaaaccccgaaaaccaacgatgataacaagacgaaagaaatcatcgatgcccagatttatctgcttaagaacatctactatggcgaatttctgaactattttatgagcaacaacggcaacttctttgaaatcagcaaagagattatcgagctgaataaaaacgacaaacgcaatctgaaaaccggcttctataaactgcagaagtttgaggatatccaagaaaagatcccgaaagaatatctggcgaatattcagagcctgtacatgattaatgcaggcaatcaggatgaggaagagaaagatacctatatcgatttcatccagaaaatctttctgaaaggctttatgacctatctggccaataatggtcgtctgagtctgatttatatcggtagtgatgaagaaaccaataccagcctggcagaaaaaaaacaagagttcgataagttcctgaagaagtacgaacagaacaacaacatcaagatcccgtatgaaatcaatgaatttctgcgcgaaatcaagctgggcaacattctgaaatacaccgaacgcctgaatatgttctatctgattctgaaactgctgaaccataaagagctgacgaatctgaaaggtagcctggaaaagtatcagagcgcaaataaagaggaagcatttagcgatcagctggaactgattaatctgctgaatctggataataaccgtgtgaccgaagatttcgaattagaagcagatgagatcggcaaattcctggattttaatggcaacaaagtgaaggacaacaaagagcttaagaagttcgacaccaacaagatctattttgatggcgagaacatcatcaaacaccgtgccttttataacatcaaaaaatacggtatgctgaacctgctggaaaagattgcagataaagcaggctataaaatcagcattgaagagttgaaaaaatacagcaacaagaaaaacgagattgagaaaaaccacaaaatgcaagaaaatctgcaccgcaaatatgcacgtccgcgtaaagatgaaaaattcaccgatgaagattatgaaagctacaaacaggccatcgaaaacatcgaagaatatacccatctgaagaacaaagtcgaattcaacgaactgaatctgctgcagggtctgctgctgcgtattctgcatcgtctggtgggttataccagcatttgggaacgtgatctgcgttttcgcctgaaaggtgaatttcctgaaaaccagtatatcgaggaaatcttcaacttcgagaataaaaagaatgtgaagtataaaggtggccagatcgtcgagaaatatatcaaattctacaaagaactgcaccagaacgacgaggtgaaaatcaacaaatatagcagcgcgaacatcaaagtgctgaaacaagagaaaaaagacctgtacatccgcaactatatcgcccactttaactatattccgcatgcagaaattagtctgctggaagttctggaaaacctgcgtaaactgctgtcatatgatcgtaaacttaaaaacgccgtgatgaaaagcgttgtggacatcctgaaagagtatggttttgttgcgacctttaaaatcggtgccgataaaaagattggtattcagaccctggaaagcgagaagattgttcacctgaaaaatcttaagaaaaagaaacttatgaccgatcgcaatagcgaggaactgtgtaaactggtgaaaattatgtttgagtataaaatggaagagaagaaatccgaaaatggggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 37 - DNA, 9907 bp, synthetic construct
  misc_feature 1..9907: note = Synthetic
  source 1..9907: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggaaggtgaccaaagttggtggtatcagccataaaaagtataccagcgaaggtcgtctggttaaaagcgaaagcgaagaaaatcgtaccgatgaacgtctgagcgcactgctgaatatgcgtctggatatgtatatcaaaaatccgagcagcaccgaaaccaaagaaaatcagaaacgtatcggcaagctgaaaaagttcttcagcaacaaaatggtgtacctgaaagataacaccctgagcctgaaaaacggcaagaaagaaaatatcgatcgcgagtatagcgaaaccgatattctggaaagtgatgtgcgtgacaaaaaaaactttgccgtcctgaaaaagatctatctgaacgaaaatgtgaacagcgaagaactggaagtgtttcgcaacgacattaaaaagaagctgaacaagatcaacagcctgaaatatagcttcgagaaaaacaaagccaactatcagaagatcaacgagaacaacatcgaaaaagtggaaggtaaaagcaagcgcaacatcatctatgattattatcgtgaaagcgccaaacgtgatgcctatgttagcaatgttaaagaggccttcgacaagctgtataaagaagaagatattgccaaactggtgctggaaattgaaaatctgaccaagctggaaaaatacaagatccgcgaattctatcacgaaatcattggtcgcaaaaacgataaagagaacttcgccaaaatcatctacgaagaaattcagaacgtgaataacatgaaagaactgatcgagaaagttccggatatgagcgaactgaaaaaaagccaggtgttctacaaatattacctggacaaagaggaactgaacgataaaaacatcaaatacgccttttgccacttcgtggaaatcgaaatgagccagctgctgaaaaactatgtgtataaacgcctgagcaacatcagcaacgataagattaaacgcatcttcgagtaccagaacctgaagaaactgattgaaaacaaactgcttaacaaactggatacctatgtgcgtaattgcggcaaatacaactattatctgcaggatggtgaaattgcgaccagcgattttattgcacgtaatcgtcagaatgaagcctttctgcgtaacattattggtgttagcagcgttgcatattttagcctgcgtaatatcctggaaaccgaaaacgagaatgatatcaccggtcgtatgcgtggtaaaaccgtgaaaaacaataaaggcgaagagaaatatgtgagcggtgaggtggataaaatctacaacgaaaacaaaaagaacgaagtgaaagaaaacctgaaaatgttttacagctacgactttaacatggacaacaagaacgagatcgaagattttttcgccaacattgatgaagccattagcagcattcgtcatggcattgttcactttaatctggaacttgagggcaaagacatcttcgcgtttaaaaacattgcaccgagcgagattagcaaaaagatgttccagaacgaaattaacgagaaaaaactgaaactgaagatctttcgccagctgaatagcgcaaatgtttttcgctatcttgagaaatacaaaatcctgaactatctgaaacgcacccgctttgaatttgtgaacaaaaacattccgtttgtgccgagctttaccaaactgtatagccgtattgatgatctgaaaaacagcctgggcatttattggaaaaccccgaaaaccaacgatgataacaagacgaaagaaatcatcgatgcccagatttatctgcttaagaacatctactatggcgaatttctgaactattttatgagcaacaacggcaacttctttgaaatcagcaaagagattatcgagctgaataaaaacgacaaacgcaatctgaaaaccggcttctataaactgcagaagtttgaggatatccaagaaaagatcccgaaagaatatctggcgaatattcagagcctgtacatgattaatgcaggcaatcaggatgaggaagagaaagatacctatatcgatttcatccagaaaatctttctgaaaggctttatgacctatctggccaataatggtcgtctgagtctgatttatatcggtagtgatgaagaaaccaataccagcctggcagaaaaaaaacaagagttcgataagttcctgaagaagtacgaacagaacaacaacatcaagatcccgtatgaaatcaatgaatttctgcgcgaaatcaagctgggcaacattctgaaatacaccgaacgcctgaatatgttctatctgattctgaaactgctgaaccataaagagctgacgaatctgaaaggtagcctggaaaagtatcagagcgcaaataaagaggaagcatttagcgatcagctggaactgattaatctgctgaatctggataataaccgtgtgaccgaagatttcgaattagaagcagatgagatcggcaaattcctggattttaatggcaacaaagtgaaggacaacaaagagcttaagaagttcgacaccaacaagatctattttgatggcgagaacatcatcaaacaccgtgccttttataacatcaaaaaatacggtatgctgaacctgctggaaaagattgcagataaagcaggctataaaatcagcattgaagagttgaaaaaatacagcaacaagaaaaacgagattgagaaaaaccacaaaatgcaagaaaatctgcaccgcaaatatgcacgtccgcgtaaagatgaaaaattcaccgatgaagattatgaaagctacaaacaggccatcgaaaacatcgaagaatatacccatctgaagaacaaagtcgaattcaacgaactgaatctgctgcagggtctgctgctgcgtattctgcatcgtctggtgggttataccagcatttgggaacgtgatctgcgttttcgcctgaaaggtgaatttcctgaaaaccagtatatcgaggaaatcttcaacttcgagaataaaaagaatgtgaagtataaaggtggccagatcgtcgagaaatatatcaaattctacaaagaactgcaccagaacgacgaggtgaaaatcaacaaatatagcagcgcgaacatcaaagtgctgaaacaagagaaaaaagacctgtacatccgcaactatatcgcccactttaactatattccgcatgcagaaattagtctgctggaagttctggaaaacctgcgtaaactgctgtcatatgatcgtaaacttaaaaacgccgtgatgaaaagcgttgtggacatcctgaaagagtatggttttgttgcgacctttaaaatcggtgccgataaaaagattggtattcagaccctggaaagcgagaagattgttcacctgaaaaatcttaagaaaaagaaacttatgaccgatcgcaatagcgaggaactgtgtaaactggtgaaaattatgtttgagtataaaatggaagagaagaaatccgaaaatgatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 38 - DNA, 9442 bp, synthetic construct
  misc_feature 1..9442: note = Synthetic
  source 1..9442: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggtaacctgtttggtcataaacgttggtatgaagtgcgcgacaaaaaagactttaaaatcaaacgcaaggtgaaagtgaaacgcaactatgatggcaacaaatatatcctgaacatcaacgagaacaacaacaaagagaagatcgataataataaattcatccgcaaatacatcaactacaaaaaaaacgataacatcctgaaagaattcacccgcaagtttcatgcaggcaacattctgtttaaactgaaaggcaaagaaggcatcattcgcatcgaaaacaatgatgattttctggaaaccgaagaggtggtgctgtatattgaagcatatggcaaaagcgaaaaactgaaggcactgggcattaccaaaaaaaagattatcgatgaagccattcgccagggtattaccaaagatgacaaaaagatcgagatcaagcgccaagaaaacgaagaagaaatcgaaattgatatccgcgacgagtataccaataaaaccctgaatgattgcagcattattctgcgcattatcgagaatgatgagctggaaacgaaaaagagcatctacgagatcttcaaaaacatcaacatgagcctgtacaaaatcatcgagaaaattatcgaaaacgaaaccgagaaggtgttcgagaatcgctattatgaagaacatctgcgtgagaaactgctgaaagatgataaaattgatgtgatcctgaccaacttcatggaaatccgcgaaaagattaaaagcaacctggaaattctgggcttcgtgaaattctatctgaatgttggtggcgacaagaaaaaaagcaagaacaagaaaatgctggtcgaaaaaattctgaacattaacgttgatctgaccgtggaagatattgccgattttgtgattaaagagctggaattctggaacatcaccaaacgcattgagaaggtgaaaaaagtgaacaacgagttcctggaaaaacgtcgtaatcgcacctatatcaaaagctatgttctgctggataagcacgagaaattcaaaattgaacgcgagaacaaaaaggacaaaatcgtgaagtttttcgtggaaaatatcaaaaacaacagcatcaaagaaaaaatcgagaagatcctggccgagttcaaaatcgatgaactgatcaaaaagctggaaaaagaactgaaaaaaggcaactgcgataccgaaattttcggcatctttaagaaacactataaagtgaacttcgatagcaaaaaattcagcaaaaagagcgacgaagagaaagagctgtataagatcatttaccgctatctgaaaggccgtattgaaaaaatcctggtgaatgaacagaaagtgcgcctgaaaaaaatggaaaaaattgagattgagaagattctgaacgagagcatcctgagtgagaaaatcctgaaacgtgttaaacagtataccctggaacacattatgtatctgggtaaactgcgccataacgatattgatatgaccaccgttaataccgatgatttcagccgtctgcatgcaaaagaagaactggatctggaactgattaccttttttgcaagcaccaatatggaactgaacaagatctttagccgtgaaaacattaacaacgacgagaacattgatttctttggtggtgatcgcgagaaaaactatgtcctggataaaaagatcctgaatagcaaaatcaagatcatccgcgatctggatttcatcgacaataagaacaacattaccaacaactttattcgcaaatttaccaaaattggcaccaatgaacgcaaccgtattctgcatgccattagcaaagaacgtgatctgcagggcacccaggatgattataacaaagtgattaacatcatccagaacctgaaaatctccgatgaagaagttagcaaagcactgaatctggatgtggtgttcaaagataagaaaaatatcatcaccaagatcaacgatatcaaaatcagcgaagagaacaataacgacatcaaatatctgccgagctttagcaaagttctgccggaaattcttaatctgtatcgcaataacccgaaaaacgaaccgtttgataccatcgaaacagagaaaattgttctgaacgccctgatctatgtgaacaaagaactgtacaagaaactgatcctggaagatgatctggaagagaacgaatcgaaaaacatctttctgcaagagctgaaaaagaccctgggtaacattgatgagatcgatgaaaacatcatcgaaaattactacaagaacgcacagattagcgcaagcaaaggtaataacaaagccatcaaaaaataccagaaaaaggtgatcgaatgctacattggttatctgcgcaaaaactacgaagaactgttcgatttcagcgatttcaaaatgaacatccaagagatcaagaagcagatcaaggacattaacgacaacaaaacctatgaacgcatcaccgttaaaaccagcgataaaaccattgtgatcaacgacgatttcgagtacatcattagcatttttgcactgctgaattccaacgccgtgatcaacaaaattcgcaatcgcttttttgccaccagtgtttggctgaataccagcgaatatcagaacattatcgatatcctggatgagatcatgcagctgaatacactgcgtaatgaatgcattaccgaaaactggaatctgaaccttgaagaatttattcagaaaatgaaagagatcgagaaagacttcgacgacttcaaaatccagaccaaaaaagaaatcttcaacaactactacgaggacatcaaaaataacattctgaccgaattcaaagacgatattaacggctgtgacgtgctggaaaagaagttggaaaagatcgttatcttcgatgacgaaaccaaattcgaaatcgacaaaaagtccaacatccttcaggatgaacagcgtaaactgagcaatatcaacaagaaagacctgaagaagaaggtcgaccagtacatcaaagacaaagaccaagaaattaagagcaaaatcctgtgccgcatcatctttaacagcgactttctgaaaaagtataagaaagagattgacaacctgatcgaggatatggaaagcgagaacgaaaacaagtttcaagagatctactatccgaaagaacgcaaaaacgagctgtacatctacaagaagaacctgttcctgaatattggcaacccgaacttcgacaaaatctatggtctgatcagcaacgacattaaaatggccgatgcaaaattcctgtttaatatcgatggtaaaaacatccgtaaaaacaaaattagcgagatcgacgcgatcctgaaaaacctgaacgataaactgaatggctacagcaaagaatataaagagaaatacattaaaaagctgaaagaaaatgacgacttcttcgccaagaacatccagaataaaaactataaaagcttcgagaaggactacaatcgcgtgtccgaatataagaaaattcgtgatctggtggaattcaactatctgaacaaaatcgaaagctatctgatcgatatcaactggaaactggcaattcagatggcacgttttgagcgtgatatgcactatattgttaatggtctgcgtgaactgggcatcattaaactgagtggttataataccggcattagccgtgcatatccgaaacgtaatggttccgatggtttttataccaccaccgcctattacaaatttttcgacgaagaaagctacaagaaatttgagaaaatttgctacggcttcggcattgatctgagcgaaaatagcgaaattaacaagccggaaaatgagagcattcgcaactatatctcccacttttatatcgtgcgtaatccgtttgccgattatagcattgcagagcagattgatcgtgttagcaatctgctgagctatagtacccgttataacaatagcacctatgccagcgtgtttgaggtgtttaaaaaggatgttaacctggactatgacgagctgaagaaaaagttcaaactgatcggcaacaatgacatcctggaacgtctgatgaaaccgaaaaaagttagtgtgctggaacttgagagctacaacagcgattatatcaagaacctgattatcgagctgctgaccaagattgaaaataccaatgataccctgggggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 39 - DNA, 10597 bp, synthetic construct
  misc_feature 1..10597: note = Synthetic
  source 1..10597: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggggtaacctgtttggtcataaacgttggtatgaagtgcgcgacaaaaaagactttaaaatcaaacgcaaggtgaaagtgaaacgcaactatgatggcaacaaatatatcctgaacatcaacgagaacaacaacaaagagaagatcgataataataaattcatccgcaaatacatcaactacaaaaaaaacgataacatcctgaaagaattcacccgcaagtttcatgcaggcaacattctgtttaaactgaaaggcaaagaaggcatcattcgcatcgaaaacaatgatgattttctggaaaccgaagaggtggtgctgtatattgaagcatatggcaaaagcgaaaaactgaaggcactgggcattaccaaaaaaaagattatcgatgaagccattcgccagggtattaccaaagatgacaaaaagatcgagatcaagcgccaagaaaacgaagaagaaatcgaaattgatatccgcgacgagtataccaataaaaccctgaatgattgcagcattattctgcgcattatcgagaatgatgagctggaaacgaaaaagagcatctacgagatcttcaaaaacatcaacatgagcctgtacaaaatcatcgagaaaattatcgaaaacgaaaccgagaaggtgttcgagaatcgctattatgaagaacatctgcgtgagaaactgctgaaagatgataaaattgatgtgatcctgaccaacttcatggaaatccgcgaaaagattaaaagcaacctggaaattctgggcttcgtgaaattctatctgaatgttggtggcgacaagaaaaaaagcaagaacaagaaaatgctggtcgaaaaaattctgaacattaacgttgatctgaccgtggaagatattgccgattttgtgattaaagagctggaattctggaacatcaccaaacgcattgagaaggtgaaaaaagtgaacaacgagttcctggaaaaacgtcgtaatcgcacctatatcaaaagctatgttctgctggataagcacgagaaattcaaaattgaacgcgagaacaaaaaggacaaaatcgtgaagtttttcgtggaaaatatcaaaaacaacagcatcaaagaaaaaatcgagaagatcctggccgagttcaaaatcgatgaactgatcaaaaagctggaaaaagaactgaaaaaaggcaactgcgataccgaaattttcggcatctttaagaaacactataaagtgaacttcgatagcaaaaaattcagcaaaaagagcgacgaagagaaagagctgtataagatcatttaccgctatctgaaaggccgtattgaaaaaatcctggtgaatgaacagaaagtgcgcctgaaaaaaatggaaaaaattgagattgagaagattctgaacgagagcatcctgagtgagaaaatcctgaaacgtgttaaacagtataccctggaacacattatgtatctgggtaaactgcgccataacgatattgatatgaccaccgttaataccgatgatttcagccgtctgcatgcaaaagaagaactggatctggaactgattaccttttttgcaagcaccaatatggaactgaacaagatctttagccgtgaaaacattaacaacgacgagaacattgatttctttggtggtgatcgcgagaaaaactatgtcctggataaaaagatcctgaatagcaaaatcaagatcatccgcgatctggatttcatcgacaataagaacaacattaccaacaactttattcgcaaatttaccaaaattggcaccaatgaacgcaaccgtattctgcatgccattagcaaagaacgtgatctgcagggcacccaggatgattataacaaagtgattaacatcatccagaacctgaaaatctccgatgaagaagttagcaaagcactgaatctggatgtggtgttcaaagataagaaaaatatcatcaccaagatcaacgatatcaaaatcagcgaagagaacaataacgacatcaaatatctgccgagctttagcaaagttctgccggaaattcttaatctgtatcgcaataacccgaaaaacgaaccgtttgataccatcgaaacagagaaaattgttctgaacgccctgatctatgtgaacaaagaactgtacaagaaactgatcctggaagatgatctggaagagaacgaatcgaaaaacatctttctgcaagagctgaaaaagaccctgggtaacattgatgagatcgatgaaaacatcatcgaaaattactacaagaacgcacagattagcgcaagcaaaggtaataacaaagccatcaaaaaataccagaaaaaggtgatcgaatgctacattggttatctgcgcaaaaactacgaagaactgttcgatttcagcgatttcaaaatgaacatccaagagatcaagaagcagatcaaggacattaacgacaacaaaacctatgaacgcatcaccgttaaaaccagcgataaaaccattgtgatcaacgacgatttcgagtacatcattagcatttttgcactgctgaattccaacgccgtgatcaacaaaattcgcaatcgcttttttgccaccagtgtttggctgaataccagcgaatatcagaacattatcgatatcctggatgagatcatgcagctgaatacactgcgtaatgaatgcattaccgaaaactggaatctgaaccttgaagaatttattcagaaaatgaaagagatcgagaaagacttcgacgacttcaaaatccagaccaaaaaagaaatcttcaacaactactacgaggacatcaaaaataacattctgaccgaattcaaagacgatattaacggctgtgacgtgctggaaaagaagttggaaaagatcgttatcttcgatgacgaaaccaaattcgaaatcgacaaaaagtccaacatccttcaggatgaacagcgtaaactgagcaatatcaacaagaaagacctgaagaagaaggtcgaccagtacatcaaagacaaagaccaagaaattaagagcaaaatcctgtgccgcatcatctttaacagcgactttctgaaaaagtataagaaagagattgacaacctgatcgaggatatggaaagcgagaacgaaaacaagtttcaagagatctactatccgaaagaacgcaaaaacgagctgtacatctacaagaagaacctgttcctgaatattggcaacccgaacttcgacaaaatctatggtctgatcagcaacgacattaaaatggccgatgcaaaattcctgtttaatatcgatggtaaaaacatccgtaaaaacaaaattagcgagatcgacgcgatcctgaaaaacctgaacgataaactgaatggctacagcaaagaatataaagagaaatacattaaaaagctgaaagaaaatgacgacttcttcgccaagaacatccagaataaaaactataaaagcttcgagaaggactacaatcgcgtgtccgaatataagaaaattcgtgatctggtggaattcaactatctgaacaaaatcgaaagctatctgatcgatatcaactggaaactggcaattcagatggcacgttttgagcgtgatatgcactatattgttaatggtctgcgtgaactgggcatcattaaactgagtggttataataccggcattagccgtgcatatccgaaacgtaatggttccgatggtttttataccaccaccgcctattacaaatttttcgacgaagaaagctacaagaaatttgagaaaatttgctacggcttcggcattgatctgagcgaaaatagcgaaattaacaagccggaaaatgagagcattcgcaactatatctcccacttttatatcgtgcgtaatccgtttgccgattatagcattgcagagcagattgatcgtgttagcaatctgctgagctatagtacccgttataacaatagcacctatgccagcgtgtttgaggtgtttaaaaaggatgttaacctggactatgacgagctgaagaaaaagttcaaactgatcggcaacaatgacatcctggaacgtctgatgaaaccgaaaaaagttagtgtgctggaacttgagagctacaacagcgattatatcaagaacctgattatcgagctgctgaccaagattgaaaataccaatgataccctggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 40 - DNA, 8731 bp, synthetic construct
  misc_feature 1..8731: note = Synthetic
  source 1..8731: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaagtgaccaaagtggatggcatcagccacaaaaaatacatcgaagaaggcaaactggttaaaagcaccagcgaagaaaatcgtaccagcgaacgtctgagcgaactgctgagcattcgtctggatatctatatcaaaaatccggataatgccagcgaggaagaaaaccgtattcgtcgtgaaaacctgaaaaagttcttcagcaataaagtgctgcacctgaaagatagcgttctgtatctgaaaaaccgcaaagaaaaaaatgccgtgcaggacaaaaactatagcgaagaggatatcagcgagtatgacctgaagaacaaaaatagctttagcgtgctgaaaaaaatcctgctgaatgaagatgtgaatagcgaggaactggaaatctttcgtaaagatgttgaagccaagctgaacaaaatcaacagcctgaaatatagctttgaagaaaacaaggccaactatcagaaaatcaacgagaacaacgtggaaaaagttggtggtaaaagcaaacgcaacatcatctatgattattatcgcgaaagcgcgaaacgcaacgattatatcaataatgtgcaagaggccttcgacaaactgtacaaaaaagaggacatcgaaaaactgttttttctgatcgagaacagcaagaagcacgagaaatacaaaatccgcgagtactaccataaaatcatcggtcgcaaaaacgataaagagaacttcgccaaaatcatctacgaagaaattcagaacgtgaacaacatcaaagaactgatcgaaaaaattccggacatgagcgagctgaagaaaagccaggtgttctataaatactacctggacaaagaggaactgaacgacaaaaacatcaaatatgccttttgccacttcgtcgaaattgaaatgagccagctgcttaaaaactacgtgtataaacgcctgagcaacatcagcaacgataaaatcaaacgtatctttgaatatcagaatctgaagaaactgattgaaaacaaactgctgaacaagctggatacctatgttcgtaattgcggcaaatacaactactatctgcaggttggtgaaattgcaaccagcgattttattgcacgtaatcgtcagaatgaagcctttctgcgtaacattattggtgttagcagcgttgcatattttagcctgcgtaatattctggaaaccgaaaacgaaaatggcattaccggtcgtatgcgtggtaaaaccgttaaaaacaataaaggcgaagagaagtatgtgagcggtgaagtggataaaatctataacgaaaacaagcagaacgaagtgaaagaaaatctgaaaatgttttacagctacgacttcaacatggacaacaaaaacgagatcgaagatttcttcgccaacattgatgaagccattagcagtattcgtcatggcattgtgcactttaatctggaacttgaaggcaaagacatcttcgcgtttaaaaacattgcaccgagcgagatcagcaaaaaaatgtttcagaacgagattaacgaaaaaaaactgaaactgaaaatcttcaaacagctgaatagcgccaacgtgttcaactattatgagaaagacgtgatcatcaaataccttaaaaacaccaaattcaacttcgtgaataaaaacatcccgtttgttccgagcttcaccaaactgtataacaaaattgaagatctgcgcaataccctgaagtttttttggagcgttccgaaagacaaagaagaaaaagacgcacagatctacctgcttaagaacatctattatggcgaatttctgaacaaattcgtgaaaaatagcaaagtgttcttcaaaatcaccaacgaggtgatcaagattaacaaacagcgtaatcagaaaaccggtcactacaaataccagaagtttgagaacattgaaaaaaccgtgccggttgaatatctggcaattattcagagccgtgagatgattaacaaccaggataaagaagagaaaaacacctacatcgatttcatccagcagatctttctgaaaggctttatcgattacctgaacaagaacaacctgaagtatatcgagtcgaacaacaataacgacaacaacgacatctttagcaaaatcaaaatcaagaaagataataaagaaaaatacgacaagatcctgaaaaactatgagaagcacaaccgcaacaaagaaattccgcatgagatcaatgaatttgtgcgcgaaattaaactgggcaaaatcctgaaatacaccgagaacctgaatatgttctatctgattctgaagctgctgaaccataaagagctgaccaatctgaaaggtagcctggaaaaatatcagagcgcaaacaaagaagagacattttctgacgaactggaactgattaatctgctgaatctggataataaccgtgtgaccgaagattttgaactggaagcaaatgaaatcggcaaattcctggatttcaatgagaacaaaattaaggaccggaaagagcttaaaaagtttgataccaacaaaatctacttcgacggcgagaacattatcaaacatcgtgccttttataacatcaaaaagtatggcatgctgaacctgctggaaaaaattgcagataaagccaagtacaaaattagcctgaaagaacttaaagagtacagcaacaaaaagaacgaaatcgagaagaactataccatgcagcagaatctgcatcgtaaatatgcacgtccgaaaaaagacgagaaattcaacgatgaggactataaagaatacgagaaagccattggcaacatccagaaatatacccacttgaaaaacaaagtggaatttaacgagctgaatttactgcagggtctgctgctgaaaattctgcaccgtctggttggttataccagcatttgggaacgtgatctgcgttttcgcctgaaaggtgaatttcctgaaaaccactatatcgaggaaattttcaactttgacaacagcaaaaacgtgaaatataagagcggtcagatcgtcgaaaagtacatcaacttttacaaagaactttacaaggataatgtggaaaaacgcagcatctacagcgacaagaaagtgaaaaagctgaagcaagaaaagaaagacctgtacatccgtaattatatcgcccactttaactatatcccgcatgcagaaattagtctgctggaagttctggaaaatctgcgtaaactgctgtcatatgatcgcaaactgaagaacgcaatcatgaaaagcattgtggatatcctgaaagagtatggttttgtcgccacctttaaaatcggtgccgataagaaaattgagattcagaccctggaaagcgagaaaattgtgcatcttaagaaccttaaaaagaaaaaactgatgaccgatcgcaacagcgaagagttatgtgaactggtgaaagtgatgttcgaatacaaagcactggaaggggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 41 - DNA, 9886 bp, synthetic construct
  misc_feature 1..9886: note = Synthetic
  source 1..9886: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggaaagtgaccaaagtggatggcatcagccacaaaaaatacatcgaagaaggcaaactggttaaaagcaccagcgaagaaaatcgtaccagcgaacgtctgagcgaactgctgagcattcgtctggatatctatatcaaaaatccggataatgccagcgaggaagaaaaccgtattcgtcgtgaaaacctgaaaaagttcttcagcaataaagtgctgcacctgaaagatagcgttctgtatctgaaaaaccgcaaagaaaaaaatgccgtgcaggacaaaaactatagcgaagaggatatcagcgagtatgacctgaagaacaaaaatagctttagcgtgctgaaaaaaatcctgctgaatgaagatgtgaatagcgaggaactggaaatctttcgtaaagatgttgaagccaagctgaacaaaatcaacagcctgaaatatagctttgaagaaaacaaggccaactatcagaaaatcaacgagaacaacgtggaaaaagttggtggtaaaagcaaacgcaacatcatctatgattattatcgcgaaagcgcgaaacgcaacgattatatcaataatgtgcaagaggccttcgacaaactgtacaaaaaagaggacatcgaaaaactgttttttctgatcgagaacagcaagaagcacgagaaatacaaaatccgcgagtactaccataaaatcatcggtcgcaaaaacgataaagagaacttcgccaaaatcatctacgaagaaattcagaacgtgaacaacatcaaagaactgatcgaaaaaattccggacatgagcgagctgaagaaaagccaggtgttctataaatactacctggacaaagaggaactgaacgacaaaaacatcaaatatgccttttgccacttcgtcgaaattgaaatgagccagctgcttaaaaactacgtgtataaacgcctgagcaacatcagcaacgataaaatcaaacgtatctttgaatatcagaatctgaagaaactgattgaaaacaaactgctgaacaagctggatacctatgttcgtaattgcggcaaatacaactactatctgcaggttggtgaaattgcaaccagcgattttattgcacgtaatcgtcagaatgaagcctttctgcgtaacattattggtgttagcagcgttgcatattttagcctgcgtaatattctggaaaccgaaaacgaaaatggtattaccggtcgtatgcgtggtaaaaccgttaaaaacaataaaggcgaagagaagtatgtgagcggtgaagtggataaaatctataacgaaaacaagcagaacgaagtgaaagaaaatctgaaaatgttttacagctacgacttcaacatggacaacaaaaacgagatcgaagatttcttcgccaacattgatgaagccattagcagtattcgtcatggcattgtgcactttaatctggaacttgaaggcaaagacatcttcgcgtttaaaaacattgcaccgagcgagatcagcaaaaaaatgtttcagaacgagattaacgaaaaaaaactgaaactgaaaatcttcaaacagctgaatagcgccaacgtgttcaactattatgagaaagacgtgatcatcaaataccttaaaaacaccaaattcaacttcgtgaataaaaacatcccgtttgttccgagcttcaccaaactgtataacaaaattgaagatctgcgcaataccctgaagtttttttggagcgttccgaaagacaaagaagaaaaagacgcacagatctacctgcttaagaacatctattatggcgaatttctgaacaaattcgtgaaaaatagcaaagtgttcttcaaaatcaccaacgaggtgatcaagattaacaaacagcgtaatcagaaaaccggtcactacaaataccagaagtttgagaacattgaaaaaaccgtgccggttgaatatctggcaattattcagagccgtgagatgattaacaaccaggataaagaagagaaaaacacctacatcgatttcatccagcagatctttctgaaaggctttatcgattacctgaacaagaacaacctgaagtatatcgagtcgaacaacaataacgacaacaacgacatctttagcaaaatcaaaatcaagaaagataataaagaaaaatacgacaagatcctgaaaaactatgagaagcacaaccgcaacaaagaaattccgcatgagatcaatgaatttgtgcgcgaaattaaactgggcaaaatcctgaaatacaccgagaacctgaatatgttctatctgattctgaagctgctgaaccataaagagctgaccaatctgaaaggtagcctggaaaaatatcagagcgcaaacaaagaagagacattttctgacgaactggaactgattaatctgctgaatctggataataaccgtgtgaccgaagattttgaactggaagcaaatgaaatcggcaaattcctggatttcaatgagaacaaaattaaggaccggaaagagcttaaaaagtttgataccaacaaaatctacttcgacggcgagaacattatcaaacatcgtgccttttataacatcaaaaagtatggcatgctgaacctgctggaaaaaattgcagataaagccaagtacaaaattagcctgaaagaacttaaagagtacagcaacaaaaagaacgaaatcgagaagaactataccatgcagcagaatctgcatcgtaaatatgcacgtccgaaaaaagacgagaaattcaacgatgaggactataaagaatacgagaaagccattggcaacatccagaaatatacccacttgaaaaacaaagtggaatttaacgagctgaatttactgcagggtctgctgctgaaaattctgcaccgtctggttggttataccagcatttgggaacgtgatctgcgttttcgcctgaaaggtgaatttcctgaaaaccactatatcgaggaaattttcaactttgacaacagcaaaaacgtgaaatataagagcggtcagatcgtcgaaaagtacatcaacttttacaaagaactttacaaggataatgtggaaaaacgcagcatctacagcgacaagaaagtgaaaaagctgaagcaagaaaagaaagacctgtacatccgtaattatatcgcccactttaactatatcccgcatgcagaaattagtctgctggaagttctggaaaatctgcgtaaactgctgtcatatgatcgcaaactgaagaacgcaatcatgaaaagcattgtggatatcctgaaagagtatggttttgtcgccacctttaaaatcggtgccgataagaaaattgagattcagaccctggaaagcgagaaaattgtgcatcttaagaaccttaaaaagaaaaaactgatgaccgatcgcaacagcgaagagttatgtgaactggtgaaagtgatgttcgaatacaaagcactggaagatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 42 - DNA, 8731 bp, synthetic construct
  misc_feature 1..8731: note = Synthetic
  source 1..8731: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaagtgaccaaagtggatggcatcagccacaaaaaatacatcgaagaaggcaaactggttaaaagcaccagcgaagaaaatcgtaccagcgaacgtctgagcgaactgctgagcattcgtctggatatctatatcaaaaatccggataatgccagcgaggaagaaaaccgtattcgtcgtgaaaacctgaaaaagttcttcagcaataaagtgctgcacctgaaagatagcgttctgtatctgaaaaaccgcaaagaaaaaaatgccgtgcaggacaaaaactatagcgaagaggatatcagcgagtatgacctgaagaacaaaaatagctttagcgtgctgaaaaaaatcctgctgaatgaagatgtgaatagcgaggaactggaaatctttcgtaaagatgttgaagccaagctgaacaaaatcaacagcctgaaatatagctttgaagaaaacaaggccaactatcagaaaatcaacgagaacaacgtggaaaaagttggtggtaaaagcaaacgcaacatcatctatgattattatcgcgaaagcgcgaaacgcaacgattatatcaataatgtgcaagaggccttcgacaaactgtacaaaaaagaggacatcgaaaaactgttttttctgatcgagaacagcaagaagcacgagaaatacaaaatccgcgagtactaccataaaatcatcggtcgcaaaaacgataaagagaacttcgccaaaatcatctacgaagaaattcagaacgtgaacaacatcaaagaactgatcgaaaaaattccggacatgagcgagctgaagaaaagccaggtgttctataaatactacctggacaaagaggaactgaacgacaaaaacatcaaatatgccttttgccacttcgtcgaaattgaaatgagccagctgcttaaaaactacgtgtataaacgcctgagcaacatcagcaacgataaaatcaaacgtatctttgaatatcagaatctgaagaaactgattgaaaacaaactgctgaacaagctggatacctatgttcgtaattgcggcaaatacaactactatctgcaggttggtgaaattgcaaccagcgattttattgcacgtaatcgtcagaatgaagcctttctgcgtaacattattggtgttagcagcgttgcatattttagcctgcgtaatattctggaaaccgaaaacgaaaatgatattaccggtcgtatgcgtggtaaaaccgttaaaaacaataaaggcgaagagaagtatgtgagcggtgaagtggataaaatctataacgaaaacaagcagaacgaagtgaaagaaaatctgaaaatgttttacagctacgacttcaacatggacaacaaaaacgagatcgaagatttcttcgccaacattgatgaagccattagcagtattcgtcatggcattgtgcactttaatctggaacttgaaggcaaagacatcttcgcgtttaaaaacattgcaccgagcgagatcagcaaaaaaatgtttcagaacgagattaacgaaaaaaaactgaaactgaaaatcttcaaacagctgaatagcgccaacgtgttcaactattatgagaaagacgtgatcatcaaataccttaaaaacaccaaattcaacttcgtgaataaaaacatcccgtttgttccgagcttcaccaaactgtataacaaaattgaagatctgcgcaataccctgaagtttttttggagcgttccgaaagacaaagaagaaaaagacgcacagatctacctgcttaagaacatctattatggcgaatttctgaacaaattcgtgaaaaatagcaaagtgttcttcaaaatcaccaacgaggtgatcaagattaacaaacagcgtaatcagaaaaccggtcactacaaataccagaagtttgagaacattgaaaaaaccgtgccggttgaatatctggcaattattcagagccgtgagatgattaacaaccaggataaagaagagaaaaacacctacatcgatttcatccagcagatctttctgaaaggctttatcgattacctgaacaagaacaacctgaagtatatcgagtcgaacaacaataacgacaacaacgacatctttagcaaaatcaaaatcaagaaagataataaagaaaaatacgacaagatcctgaaaaactatgagaagcacaaccgcaacaaagaaattccgcatgagatcaatgaatttgtgcgcgaaattaaactgggcaaaatcctgaaatacaccgagaacctgaatatgttctatctgattctgaagctgctgaaccataaagagctgaccaatctgaaaggtagcctggaaaaatatcagagcgcaaacaaagaagagacattttctgacgaactggaactgattaatctgctgaatctggataataaccgtgtgaccgaagattttgaactggaagcaaatgaaatcggcaaattcctggatttcaatgagaacaaaattaaggaccggaaagagcttaaaaagtttgataccaacaaaatctacttcgacggcgagaacattatcaaacatcgtgccttttataacatcaaaaagtatggcatgctgaacctgctggaaaaaattgcagataaagccaagtacaaaattagcctgaaagaacttaaagagtacagcaacaaaaagaacgaaatcgagaagaactataccatgcagcagaatctgcatcgtaaatatgcacgtccgaaaaaagacgagaaattcaacgatgaggactataaagaatacgagaaagccattggcaacatccagaaatatacccacttgaaaaacaaagtggaatttaacgagctgaatttactgcagggtctgctgctgaaaattctgcaccgtctggttggttataccagcatttgggaacgtgatctgcgttttcgcctgaaaggtgaatttcctgaaaaccactatatcgaggaaattttcaactttgacaacagcaaaaacgtgaaatataagagcggtcagatcgtcgaaaagtacatcaacttttacaaagaactttacaaggataatgtggaaaaacgcagcatctacagcgacaagaaagtgaaaaagctgaagcaagaaaagaaagacctgtacatccgtaattatatcgcccactttaactatatcccgcatgcagaaattagtctgctggaagttctggaaaatctgcgtaaactgctgtcatatgatcgcaaactgaagaacgcaatcatgaaaagcattgtggatatcctgaaagagtatggttttgtcgccacctttaaaatcggtgccgataagaaaattgagattcagaccctggaaagcgagaaaattgtgcatcttaagaaccttaaaaagaaaaaactgatgaccgatcgcaacagcgaagagttatgtgaactggtgaaagtgatgttcgaatacaaagcactggaaggggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 43 - DNA, 5206 bp, synthetic construct
  misc_feature 1..5206: note = Synthetic
  source 1..5206: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccgatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 44 - DNA, 6367 bp, synthetic construct
  misc_feature 1..6367: note = Synthetic
  source 1..6367: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaagggatccggctgctaacaaagcccgaaaggaagctgagttggctgctgccaccgctgagcaataactagcataaccccttggggcctctaaacgggtcttgaggggttttttgctgaaaggaggaactatatccggat